SEQUENCE LISTING (105 sequences)

SEQ ID NO: 1 - protein, 5 aa, synthetic construct
GGGGS

SEQ ID NO: 2 - protein, 10 aa, synthetic construct
GGGGSGGGGS

SEQ ID NO: 3 - protein, 15 aa, synthetic construct
GGGGSGGGGSGGGGS

SEQ ID NO: 4 - protein, 45 aa, synthetic construct
TTTPAPRPPTPAPTIASQPLSLRPEACRPAAGGAVHTRGLDFACD

SEQ ID NO: 5 - protein, 42 aa, synthetic construct
AAAIEVMYPPPYLDNEKSNGTIIHVKGKHLCPSPLFPGPSKP

SEQ ID NO: 6 - protein, 24 aa, synthetic construct
IYIWAPLAGTCGVLLLSLVITLYC

SEQ ID NO: 7 - protein, 68 aa, synthetic construct
FWVLVVVGGVLACYSLLVTVAFIIFWVRSKRSRLLHSDYMNMTPRRPGPTRKHYQPYAPPRDFAAYRS

SEQ ID NO: 8 - protein, 112 aa, synthetic construct
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 9 - protein, 112 aa, synthetic construct
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 10 - protein, 42 aa, synthetic construct
KRGRKKLLYIFKQPFMRPVQTTQEEDGCSCRFPEEEEGGCEL

SEQ ID NO: 11 - protein, 217 aa, synthetic construct
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 11 aa, synthetic construct
GGNNIGSKNVH

SEQ ID NO: 13 - protein, 7 aa, synthetic construct
RDSNRPS

SEQ ID NO: 14 - protein, 9 aa, synthetic construct
QVWDSSTVV

SEQ ID NO: 15 - protein, 10 aa, synthetic construct
GYTFTSYGIS

SEQ ID NO: 16 - protein, 17 aa, synthetic construct
WISAYNGNTNYAQKLQG

SEQ ID NO: 17 - protein, 6 aa, synthetic construct
GYAFDI

SEQ ID NO: 18 - protein, 11 aa, synthetic construct
RASQSISSYLN

SEQ ID NO: 19 - protein, 7 aa, synthetic construct
AASSLQS

SEQ ID NO: 20 - protein, 10 aa, synthetic construct
QQSYSTPPWT

SEQ ID NO: 21 - protein, 10 aa, synthetic construct
GFTFSSYEMN

SEQ ID NO: 22 - protein, 17 aa, synthetic construct
AISGGGGSTYYADSVKG

SEQ ID NO: 23 - protein, 10 aa, synthetic construct
SGRGWAAAGR

SEQ ID NO: 24 - protein, 10 aa, synthetic construct
DFSFDSYEMS

SEQ ID NO: 25 - protein, 17 aa, synthetic construct
SISASGGRTFYADSVKG

SEQ ID NO: 26 - protein, 4 aa, synthetic construct
HPGI

SEQ ID NO: 27 - protein, 10 aa, synthetic construct
SFDFASYEMS

SEQ ID NO: 28 - protein, 17 aa, synthetic construct
SISGSGGHIFYADSVKG

SEQ ID NO: 29 - skipped

SEQ ID NO: 30 - protein, 11 aa, synthetic construct
GGNNIGSKNVH

SEQ ID NO: 31 - protein, 7 aa, synthetic construct
RDSNRPS

SEQ ID NO: 32 - protein, 9 aa, synthetic construct
QVWDSSTGV

SEQ ID NO: 33 - protein, 10 aa, synthetic construct
GYTFTSYGIS

SEQ ID NO: 34 - protein, 17 aa, synthetic construct
WISAYNGNTNYAQKLQG

SEQ ID NO: 35 - protein, 6 aa, synthetic construct
GYSYGL

SEQ ID NO: 36 - protein, 10 aa, synthetic construct
DFSFSAYEMS

SEQ ID NO: 37 - protein, 17 aa, synthetic construct
SISGSGRSTYYADSVKG

SEQ ID NO: 38 - protein, 11 aa, synthetic construct
GSGYYDSSGYL

SEQ ID NO: 39 - protein, 11 aa, synthetic construct
QGDSLRTYYAS

SEQ ID NO: 40 - protein, 7 aa, synthetic construct
GKNNRPS

SEQ ID NO: 41 - protein, 9 aa, synthetic construct
QTWDSTTGV

SEQ ID NO: 42 - protein, 10 aa, synthetic construct
GFTFSNYEMN

SEQ ID NO: 43 - protein, 17 aa, synthetic construct
AISGSGGSTYYADSVKG

SEQ ID NO: 44 - protein, 10 aa, synthetic construct
DGGGWFGEEY

SEQ ID NO: 45 - protein, 10 aa, synthetic construct
AFAFADYEMS

SEQ ID NO: 46 - protein, 17 aa, synthetic construct
AISGSGGSTYYADSVKG

SEQ ID NO: 47 - protein, 10 aa, synthetic construct
GTAYGDYENY

SEQ ID NO: 48 - protein, 10 aa, synthetic construct
AFDFDDAYMD

SEQ ID NO: 49 - protein, 17 aa, synthetic construct
GINWNSGWVGYADSVKG

SEQ ID NO: 50 - protein, 12 aa, synthetic construct
TGIAAAGTNFDY

SEQ ID NO: 51 - protein, 13 aa, synthetic construct
SGSSSNIGNNYVS

SEQ ID NO: 52 - protein, 7 aa, synthetic construct
DNNKRPS

SEQ ID NO: 53 - protein, 11 aa, synthetic construct
GTWDSSLSAWV

SEQ ID NO: 54 - protein, 10 aa, synthetic construct
GYTFTGYYMH

SEQ ID NO: 55 - protein, 17 aa, synthetic construct
IINPSGGSTIYAQKFQG

SEQ ID NO: 56 - protein, 12 aa, synthetic construct
VKYSKLNDAFDI

SEQ ID NO: 57 - protein, 10 aa, synthetic construct
SFAFDSYEMS

SEQ ID NO: 58 - protein, 17 aa, synthetic construct
SISGSGGSTYYADSVKG

SEQ ID NO: 59 - skipped

SEQ ID NO: 60 - DNA, 318 bp, synthetic construct
  source 1..318: mol_type = other DNA
tcctatgagctgacacagccactctcagtgtcagtggccctgggacagacggccaggattacctgtgggggaaacaacattggaagtaaaaatgtgcactggtaccagcagaagccaggccaggcccctgtgctggtcatctatagggatagcaaccggccctctgggatccctgagcgattctctggctccaactcggggaacacggccaccctgaccatcaacagagcccaagccggggatgaggctgactattactgtcaggtgtgggacagcagcactgtggtattcggcggagggacggagctgaccgtccta

SEQ ID NO: 61 - protein, 106 aa, synthetic construct
SYELTQPLSVSVALGQTARITCGGNNIGSKNVHWYQQKPGQAPVLVIYRDSNRPSGIPERFSGSNSGNTATLTINRAQAGDEADYYCQVWDSSTVVFGGGTELTVL

SEQ ID NO: 62 - DNA, 345 bp, synthetic construct
  source 1..345: mol_type = other DNA
caggttcagctggtgcagtctggagctgaggtgaagaagcctggggcctcagtgaaggtctcctgcaaggcttctggttacacctttaccagctatggtatcagctgggtgcgacaggcccctggacaagggcttgagtggatgggatggatcagcgcttacaatggtaacacaaactatgcacagaagctccagggcagagtcaccatgaccacagacacatccacgagcacagcctacatggagctgaggagcctgagatctgacgacacggccgtgtattactgtgcgagagggtatgcttttgatatctggggccaagggacaatggtcaccgtctcctct

SEQ ID NO: 63 - protein, 115 aa, synthetic construct
QVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCARGYAFDIWGQGTMVTVSS

SEQ ID NO: 64 - DNA, 708 bp, synthetic construct
  source 1..708: mol_type = other DNA
tcctatgagctgacacagccactctcagtgtcagtggccctgggacagacggccaggattacctgtgggggaaacaacattggaagtaaaaatgtgcactggtaccagcagaagccaggccaggcccctgtgctggtcatctatagggatagcaaccggccctctgggatccctgagcgattctctggctccaactcggggaacacggccaccctgaccatcaacagagcccaagccggggatgaggctgactattactgtcaggtgtgggacagcagcactgtggtattcggcggagggacggagctgaccgtcctaggtggaggcggttccggaggcggtggcagcggcggtggcggttcgcaggttcagctggtgcagtctggagctgaggtgaagaagcctggggcctcagtgaaggtctcctgcaaggcttctggttacacctttaccagctatggtatcagctgggtgcgacaggcccctggacaagggcttgagtggatgggatggatcagcgcttacaatggtaacacaaactatgcacagaagctccagggcagagtcaccatgaccacagacacatccacgagcacagcctacatggagctgaggagcctgagatctgacgacacggccgtgtattactgtgcgagagggtatgcttttgatatctggggccaagggacaatggtcaccgtctcctca

SEQ ID NO: 65 - protein, 236 aa, synthetic construct
SYELTQPLSVSVALGQTARITCGGNNIGSKNVHWYQQKPGQAPVLVIYRDSNRPSGIPERFSGSNSGNTATLTINRAQAGDEADYYCQVWDSSTVVFGGGTELTVLGGGGSGGGGSGGGGSQVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCARGYAFDIWGQGTMVTVSS

SEQ ID NO: 66 - DNA, 324 bp, synthetic construct
  source 1..324: mol_type = other DNA
gccatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcaacagagttacagtacccctccgtggacgttcggccaagggaccaaggtggagatcaaa

SEQ ID NO: 67 - protein, 108 aa, synthetic construct
AIQMTQSPSSLSASVGDRVTITCRASQSISSYLNWYQQKPGKAPKLLIYAASSLQSGVPSRFSGSGSGTDFTLTISSLQPEDFATYYCQQSYSTPPWTFGQGTKVEIK

SEQ ID NO: 68 - DNA, 357 bp, synthetic construct
  source 1..357: mol_type = other DNA
caggtgcagccggtggagtctgggggaggcttggtacagcctggagggtccctgagactctcctgtgcagcctctggattcaccttcagtagttatgaaatgaactgggtccgccaggctccagggaaggggctggagtgggtctcagctattagtggtggtggtggtagcacatactacgcagactccgtgaagggccggttcaccatctccagagacaattccaagaacacgctgtatctgcagatgaacagcctgagagccgaggacacggccgtatattactgtgcgaaatccgggcgcgggtgggcagcagctggtagatggggccagggcaccctggtcaccgtctcctca

SEQ ID NO: 69 - protein, 119 aa, synthetic construct
QVQPVESGGGLVQPGGSLRLSCAASGFTFSSYEMNWVRQAPGKGLEWVSAISGGGGSTYYADSVKGRFTISRDNSKNTLYLQMNSLRAEDTAVYYCAKSGRGWAAAGRWGQGTLVTVSS

SEQ ID NO: 70 - DNA, 726 bp, synthetic construct
  source 1..726: mol_type = other DNA
gccatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcaacagagttacagtacccctccgtggacgttcggccaagggaccaaggtggagatcaaaggtggaggcggttccggaggcggtggcagcggcggtggcggttcgcaggtgcagccggtggagtctgggggaggcttggtacagcctggagggtccctgagactctcctgtgcagcctctggattcaccttcagtagttatgaaatgaactgggtccgccaggctccagggaaggggctggagtgggtctcagctattagtggtggtggtggtagcacatactacgcagactccgtgaagggccggttcaccatctccagagacaattccaagaacacgctgtatctgcagatgaacagcctgagagccgaggacacggccgtatattactgtgcgaaatccgggcgcgggtgggcagcagctggtagatggggccagggcaccctggtcaccgtctcctca

SEQ ID NO: 71 - protein, 242 aa, synthetic construct
AIQMTQSPSSLSASVGDRVTITCRASQSISSYLNWYQQKPGKAPKLLIYAASSLQSGVPSRFSGSGSGTDFTLTISSLQPEDFATYYCQQSYSTPPWTFGQGTKVEIKGGGGSGGGGSGGGGSQVQPVESGGGLVQPGGSLRLSCAASGFTFSSYEMNWVRQAPGKGLEWVSAISGGGGSTYYADSVKGRFTISRDNSKNTLYLQMNSLRAEDTAVYYCAKSGRGWAAAGRWGQGTLVTVSS

SEQ ID NO: 72 - DNA, 339 bp, synthetic construct
  source 1..339: mol_type = other DNA
caggtgcagctggtgcagtctgggggaggcttggtacagcctggagggtccctgagactctcctgtgcagcctctgatttctctttcgattcttatgaaatgagctgggtccgccaggctccagggaaggggctagagtgggtctcaagtattagtgctagtggtggtaggacattctacgcagactctgtgaagggccgattcaccatctccagagacaattccaagaacacgctgtatctgcaaatgaacaccctgagagccgaggacacagccttgtattactgtgcgagacatccggggattatgggccagggaaccctggtcaccgtctcctca

SEQ ID NO: 73 - protein, 113 aa, synthetic construct
QVQLVQSGGGLVQPGGSLRLSCAASDFSFDSYEMSWVRQAPGKGLEWVSSISASGGRTFYADSVKGRFTISRDNSKNTLYLQMNTLRAEDTALYYCARHPGIMGQGTLVTVSS

SEQ ID NO: 74 - DNA, 336 bp, synthetic construct
  source 1..336: mol_type = other DNA
caggtgcagctggtgcagtctgggggaggcttggtacagcctggagggtccctgagactctcctgtgcagcctcttctttcgatttcgcttcttatgaaatgagctgggtccgccaggctccagggaagggactagagtgggtctcatcgattagtggtagtggtggtcacatattctacgcagactccgtgaagggccgattcaccatctccagagacaattccaagaacacgctgtatctgcaaatgaacaccctgagagccgaggacacggccgtgtattactgtgcgagagatggattgatgggccagggaaccctggtcaccgtctcctca

SEQ ID NO: 75 - protein, 112 aa, synthetic construct
QVQLVQSGGGLVQPGGSLRLSCAASSFDFASYEMSWVRQAPGKGLEWVSSISGSGGHIFYADSVKGRFTISRDNSKNTLYLQMNTLRAEDTAVYYCARDGLMGQGTLVTVSS

SEQ ID NO: 76 - DNA, 318 bp, synthetic construct
  source 1..318: mol_type = other DNA
tcctatgtgctgactcagccactctcagtgtcagtggccctgggacagacggccaggattacctgtgggggaaacaacattggaagtaaaaatgtgcactggtaccagcagaagccaggccaggcccctgtgctggtcatctatagggatagcaaccggccctctgggatccctgagcgattctctggctccaactcggggaacacggccatcctgaccatcagcagagcccaagccggggatgaggctgactattactgtcaggtgtgggacagcagcactggggtgttcggcggaggaacccagctgatcatttta

SEQ ID NO: 77 - protein, 106 aa, synthetic construct
SYVLTQPLSVSVALGQTARITCGGNNIGSKNVHWYQQKPGQAPVLVIYRDSNRPSGIPERFSGSNSGNTAILTISRAQAGDEADYYCQVWDSSTGVFGGGTQLIIL

SEQ ID NO: 78 - DNA, 345 bp, synthetic construct
  source 1..345: mol_type = other DNA
caggtgcagctggtgcagtctggagctgaggtgaagaagcctggggcctcagtgaaggtctcctgcaaggcttctggttacacctttaccagctatggtatcagctgggtgcgacaggcccctggacaagggcttgagtggatgggatggatcagcgcttacaatggtaacacaaactatgcacagaagctccagggcagggtcaccatgaccacagacacatccacgagcacagcctacatggagctgaggagcctgagatctgacgacacggccgtgtattactgtgcgagagggtacagctatggtctctggggccagggaaccacggtcaccgtctcctca

SEQ ID NO: 79 - protein, 115 aa, synthetic construct
QVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCARGYSYGLWGQGTTVTVSS

SEQ ID NO: 80 - DNA, 708 bp, synthetic construct
  source 1..708: mol_type = other DNA
tcctatgtgctgactcagccactctcagtgtcagtggccctgggacagacggccaggattacctgtgggggaaacaacattggaagtaaaaatgtgcactggtaccagcagaagccaggccaggcccctgtgctggtcatctatagggatagcaaccggccctctgggatccctgagcgattctctggctccaactcggggaacacggccatcctgaccatcagcagagcccaagccggggatgaggctgactattactgtcaggtgtgggacagcagcactggggtgttcggcggaggaacccagctgatcattttaggaggaggcggttccggaggcggtggcagcggcggtggcggttcgcaggtgcagctggtgcagtctggagctgaggtgaagaagcctggggcctcagtgaaggtctcctgcaaggcttctggttacacctttaccagctatggtatcagctgggtgcgacaggcccctggacaagggcttgagtggatgggatggatcagcgcttacaatggtaacacaaactatgcacagaagctccagggcagggtcaccatgaccacagacacatccacgagcacagcctacatggagctgaggagcctgagatctgacgacacggccgtgtattactgtgcgagagggtacagctatggtctctggggccagggaaccacggtcaccgtctcctca

SEQ ID NO: 81 - protein, 236 aa, synthetic construct
SYVLTQPLSVSVALGQTARITCGGNNIGSKNVHWYQQKPGQAPVLVIYRDSNRPSGIPERFSGSNSGNTAILTISRAQAGDEADYYCQVWDSSTGVFGGGTQLIILGGGGSGGGGSGGGGSQVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCARGYSYGLWGQGTTVTVSS

SEQ ID NO: 82 - DNA, 360 bp, synthetic construct
  source 1..360: mol_type = other DNA
caggtgcagctggtgcagtctgggggaggcctggtacagcctggagggtccctgagactctcctgtgcagcctctgatttctctttctctgcttatgaaatgagctgggtccgccaggctccagggaagggcctagagtgggtctcaagtattagtggtagtggtcgtagcacatactacgcagactccgtgaagggccgattcaccatctccagagacaattccaagaacacgctgtatctgcaaatgaacaccctgagagccgaggacacagccatgtattactgtgcgaaaggtagcggttactatgatagtagtggttacctctggggccagggaaccctggtcaccgtctcctca

SEQ ID NO: 83 - protein, 120 aa, synthetic construct
QVQLVQSGGGLVQPGGSLRLSCAASDFSFSAYEMSWVRQAPGKGLEWVSSISGSGRSTYYADSVKGRFTISRDNSKNTLYLQMNTLRAEDTAMYYCAKGSGYYDSSGYLWGQGTLVTVSS

SEQ ID NO: 84 - DNA, 318 bp, synthetic construct
  source 1..318: mol_type = other DNA
tcttctgagctgactcaggaccctgctgtgtctgtggccttgggacagacagtcaggatcacatgccaaggagacagcctcagaacctattatgcaagctggtaccagcagaagccaggacaggcccctgtacttgtcatctatggtaaaaacaaccggccctcagggatccctgagcgattctctggctccaactctgggaacatagccactctgaccatcagcgggacccagcctatggatgaggctgactattactgtcagacgtgggacagtaccactggcgtcttcggcggagggaccgagctgaccgtccta

SEQ ID NO: 85 - protein, 106 aa, synthetic construct
SSELTQDPAVSVALGQTVRITCQGDSLRTYYASWYQQKPGQAPVLVIYGKNNRPSGIPERFSGSNSGNIATLTISGTQPMDEADYYCQTWDSTTGVFGGGTELTVL

SEQ ID NO: 86 - DNA, 357 bp, synthetic construct
  source 1..357: mol_type = other DNA
caggtgcagctggtggagtctgggggaggcttggtacagcctggagggtccctgagactctcctgtgcaacctctggattcaccttcagtaattatgaaatgaactgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccgtatattactgtgcgaaagatgggggcggttggttcggggaggagtactggggccagggagccacggtcaccgtctcctca

SEQ ID NO: 87 - protein, 119 aa, synthetic construct
QVQLVESGGGLVQPGGSLRLSCATSGFTFSNYEMNWVRQAPGKGLEWVSAISGSGGSTYYADSVKGRFTISRDNSKNTLYLQMNSLRAEDTAVYYCAKDGGGWFGEEYWGQGATVTVSS

SEQ ID NO: 88 - DNA, 720 bp, synthetic construct
  source 1..720: mol_type = other DNA
tcttctgagctgactcaggaccctgctgtgtctgtggccttgggacagacagtcaggatcacatgccaaggagacagcctcagaacctattatgcaagctggtaccagcagaagccaggacaggcccctgtacttgtcatctatggtaaaaacaaccggccctcagggatccctgagcgattctctggctccaactctgggaacatagccactctgaccatcagcgggacccagcctatggatgaggctgactattactgtcagacgtgggacagtaccactggcgtcttcggcggagggaccgagctgaccgtcctaggtggaggcggttccggaggcggtggcagcggcggtggcggttcgcaggtgcagctggtggagtctgggggaggcttggtacagcctggagggtccctgagactctcctgtgcaacctctggattcaccttcagtaattatgaaatgaactgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccgtatattactgtgcgaaagatgggggcggttggttcggggaggagtactggggccagggagccacggtcaccgtctcctca

SEQ ID NO: 89 - protein, 240 aa, synthetic construct
SSELTQDPAVSVALGQTVRITCQGDSLRTYYASWYQQKPGQAPVLVIYGKNNRPSGIPERFSGSNSGNIATLTISGTQPMDEADYYCQTWDSTTGVFGGGTELTVLGGGGSGGGGSGGGGSQVQLVESGGGLVQPGGSLRLSCATSGFTFSNYEMNWVRQAPGKGLEWVSAISGSGGSTYYADSVKGRFTISRDNSKNTLYLQMNSLRAEDTAVYYCAKDGGGWFGEEYWGQGATVTVSS

SEQ ID NO: 90 - DNA, 357 bp, synthetic construct
  source 1..357: mol_type = other DNA
caggtgcagctggtgcagtctgggggaggcttggtacagcctggagggtccctgagactctcctgtgcagcctctgctttcgctttcgctgattatgaaatgagctgggtccgccaggctccaggacaaggccttgagtgggtctcagctattagtggtagtggtggtagcacatactacgcagactccgtgaagggccgatttaccatctccagagacaattccaagaacacgctgtatctgcaaatgaacaccctgagagccgaggacacggccgtgtattactgtgcgagaggcacagcctacggtgactacgaaaactactggggccagggaaccctggtcaccgtctcctca

SEQ ID NO: 91 - protein, 119 aa, synthetic construct
QVQLVQSGGGLVQPGGSLRLSCAASAFAFADYEMSWVRQAPGQGLEWVSAISGSGGSTYYADSVKGRFTISRDNSKNTLYLQMNTLRAEDTAVYYCARGTAYGDYENYWGQGTLVTVSS

SEQ ID NO: 92 - DNA, 363 bp, synthetic construct
  source 1..363: mol_type = other DNA
caggtgcagctggtgcagtctgggggaggcttggtacagcctggagggtccctgagactctcctgtgcagcctctgctttcgatttcgatgatgcttatatggactgggtccgccaggctccagggaaggggctagagtgggtcgcaggtattaattggaatagtggttgggtaggctatgcggactcagtgaagggccgattcaccatctccagagacaattccaagaacacgctgtatctgcaaatgaacaccctgagagccgaggacacggccgtatattactgtgcgagaacgggtatagcagcagctggtaccaactttgactactggggccagggaaccctggtcactgtctcctca

SEQ ID NO: 93 - protein, 121 aa, synthetic construct
QVQLVQSGGGLVQPGGSLRLSCAASAFDFDDAYMDWVRQAPGKGLEWVAGINWNSGWVGYADSVKGRFTISRDNSKNTLYLQMNTLRAEDTAVYYCARTGIAAAGTNFDYWGQGTLVTVSS

SEQ ID NO: 94 - DNA, 330 bp, synthetic construct
  source 1..330: mol_type = other DNA
cagtctgtgttgactcagccgccctcagtgtctgcggccccaggacagaaggtcaccatctcctgctctggaagcagctccaacattgggaataattatgtttcctggtaccagcacctcccaggaacagcccccaaactcctcatttatgacaataataagcgaccctcagggattcctgaccgattctctggctccaagtctgccacgtcagccaccctgggcatcaccggactccagactggggacgaggccgattattactgcggaacatgggatagcagcctgagtgcttgggtgttcggcggagggaccaagctgaccgtccta

SEQ ID NO: 95 - protein, 110 aa, synthetic construct
QSVLTQPPSVSAAPGQKVTISCSGSSSNIGNNYVSWYQHLPGTAPKLLIYDNNKRPSGIPDRFSGSKSATSATLGITGLQTGDEADYYCGTWDSSLSAWVFGGGTKLTVL

SEQ ID NO: 96 - DNA, 363 bp, synthetic construct
  source 1..363: mol_type = other DNA
caggtgcagctggtgcagtctggggctgaggtgaagaagcctggggcctcagtgaaggtctcctgcaaggcttctggatacaccttcaccggctactatatgcactgggtgcgacaggcccctggacaagggcttgagtggatgggaataatcaaccctagtggtggtagcacaatctacgcacagaagttccagggcagagtcaccatgaccagggacacgtccacgagcacagtctacatggagctgagcagcctgggatctgaggacacggccgtgtattactgtgcgagagtaaaatacagcaagcttaatgatgcttttgatatctggggccaagggacaatggtcaccgtctcttca

SEQ ID NO: 97 - protein, 121 aa, synthetic construct
QVQLVQSGAEVKKPGASVKVSCKASGYTFTGYYMHWVRQAPGQGLEWMGIINPSGGSTIYAQKFQGRVTMTRDTSTSTVYMELSSLGSEDTAVYYCARVKYSKLNDAFDIWGQGTMVTVSS

SEQ ID NO: 98 - DNA, 738 bp, synthetic construct
  source 1..738: mol_type = other DNA
cagtctgtgttgactcagccgccctcagtgtctgcggccccaggacagaaggtcaccatctcctgctctggaagcagctccaacattgggaataattatgtttcctggtaccagcacctcccaggaacagcccccaaactcctcatttatgacaataataagcgaccctcagggattcctgaccgattctctggctccaagtctgccacgtcagccaccctgggcatcaccggactccagactggggacgaggccgattattactgcggaacatgggatagcagcctgagtgcttgggtgttcggcggagggaccaagctgaccgtcctaggtggaggcggttccggaggcggtggcagcggcggtggcggttcgcaggtgcagctggtgcagtctggggctgaggtgaagaagcctggggcctcagtgaaggtctcctgcaaggcttctggatacaccttcaccggctactatatgcactgggtgcgacaggcccctggacaagggcttgagtggatgggaataatcaaccctagtggtggtagcacaatctacgcacagaagttccagggcagagtcaccatgaccagggacacgtccacgagcacagtctacatggagctgagcagcctgggatctgaggacacggccgtgtattactgtgcgagagtaaaatacagcaagcttaatgatgcttttgatatctggggccaagggacaatggtcaccgtctcttca

SEQ ID NO: 99 - protein, 246 aa, synthetic construct
QSVLTQPPSVSAAPGQKVTISCSGSSSNIGNNYVSWYQHLPGTAPKLLIYDNNKRPSGIPDRFSGSKSATSATLGITGLQTGDEADYYCGTWDSSLSAWVFGGGTKLTVLGGGGSGGGGSGGGGSQVQLVQSGAEVKKPGASVKVSCKASGYTFTGYYMHWVRQAPGQGLEWMGIINPSGGSTIYAQKFQGRVTMTRDTSTSTVYMELSSLGSEDTAVYYCARVKYSKLNDAFDIWGQGTMVTVSS

SEQ ID NO: 100 - DNA, 336 bp, synthetic construct
  source 1..336: mol_type = other DNA
caggtgcagctggtgcagtctgggggaggcttggtacagcctggagggtccctgagactctcctgtgcagcctcttctttcgctttcgattcttatgaaatgagctgggtccgccaggctccaggacaaggccttgagtgggtctcatctattagtggtagtggtggtagcacatactacgcagactccgtgaagggccgattcaccatctccagagacaattccaagaacacgctgtatctgcaaatgaacaccctgagagccgaggacacggccgtatattactgtgcgagagattcccttatgggccagggaaccctggtcaccgtctcctca

SEQ ID NO: 101 - protein, 112 aa, synthetic construct
QVQLVQSGGGLVQPGGSLRLSCAASSFAFDSYEMSWVRQAPGQGLEWVSSISGSGGSTYYADSVKGRFTISRDNSKNTLYLQMNTLRAEDTAVYYCARDSLMGQGTLVTVSS

SEQ ID NO: 102 - DNA, 45 bp, synthetic construct
  source 1..45: mol_type = other DNA
ggtggaggcggttccggaggcggtggcagcggcggtggcggttcg

SEQ ID NO: 103 - DNA, 45 bp, synthetic construct
  source 1..45: mol_type = other DNA
ggaggaggcggttccggaggcggtggcagcggcggtggcggttcg

SEQ ID NO: 104 - RNA, 20 nt, synthetic construct
  source 1..20: mol_type = other RNA
cttggccaataacaccatcc

SEQ ID NO: 105 - RNA, 20 nt, synthetic construct
  source 1..20: mol_type = other RNA
gtggtacctgctggctgagg

(19) United States
(12) Patent Application Publication
(10) Pub. No.: US 2026/0356970 A1
(43) Pub. Date: Nov. 12, 2026

(54) EGF-LIKE MODULE CONTAINING MUCIN-LIKE HORMONE-LIKE 2 (ERM2) BINDING AGENTS AND METHODS OF USE THEREOF

(71) Applicant: SYZYGYMED INC., Anacortes, WA (US)

(72) Inventors: Julian Scherer, Cambridge, MA (US); Mariana Araujo Alves Martins Da Silva, Cambridge, MA (US); Tirtha Chakraborty, Cambridge, MA (US)

(73) Assignee: SYZYGYMED INC., Anacortes, WA (US)

(21) Appl. No.: 19/484,050

(22) PCT Filed: May 14, 2024
(86) PCT No.: PCT/US2024/029308

§ 371 Date: Nov. 13, 2025

Related U.S. Application Data
(60) Provisional application No. 63/466,583, filed on May 15, 2023.

(51) Int. Cl. C07K 16/28; A61K 40/11; A61K 40/31; A61K 40/42; A61K 45/06; A61P 35/00; A61P 35/02; C12N 5/0783

(52) U.S. Cl. CPC C07K 16/2896; A61K 40/11; A61K 40/31; A61K 40/4224; A61K 45/06; A61P 35/00; A61P 35/02; C12N 5/0636; C07K 2317/56; C07K 2317/569; C07K 2317/622; C07K 2317/92; C12N 2510/00

(57) ABSTRACT

The present disclosure provides antibodies, antigen binding fragments thereof, and fusion proteins, including chimeric antigen receptors, that target EGF-like module-containing mucin-like hormone receptor-like 2 (EMR2). Also provided herein are methods of using such antibodies, antigen binding fragments thereof, and fusion proteins, for treating hematopoietic malignancies or hematological diseases (e.g., acute myeloid leukemia, myelodysplastic syndrome).

### RELATED APPLICATIONS

The application claims the benefit under 35 U.S.C. 119(e) of U.S. Provisional Application No. 63/466,583 filed May 15, 2023 which is incorporated by reference in its entirety.

### REFERENCE TO AN ELECTRONIC SEQUENCE LISTING

The contents of the electronic sequence listing (V029170044WO00-SEQ-CEW.xml; Size: 103,220 bytes; and Date of Creation: May 13, 2024) is herein incorporated by reference in its entirety.

### BACKGROUND

EGF-like module-containing mucin-like hormone receptor-like 2 (EMR2), also known as CD312, is a member of the EGF-seven-span transmembrane (TM7) family of adhesion G protein-coupled receptors (GPCR), expression of which has been associated with hematopoietic disorders, i.e., relapsed/refractory acute myeloid leukemia (AML). AML is a disease resulting in uncontrollable accumulation of immature myeloid blasts in the bone marrow and peripheral blood, and the disease has multiple subtypes that contribute to the challenge in developing an encompassing targeted therapy. Chimeric Antigen Receptor (CAR) T-cell therapy has shown promising clinical outcomes for patients with AML, although development of targeted CAR T-cell therapies still remain challenging.

### SUMMARY

In some aspects, the present disclosure provides anti-EMR2 antibodies, or antigen-binding fragments thereof, comprising an amino acid sequence of any one of SEQ ID NOs: 65, 71, 73, 75, 81, 83, 89, 91, 93, 99, 101.

In some aspects, the present disclosure provides anti-EMR2 antibodies, or antigen-binding fragments thereof, comprising at least one complementarity determining region (CDR) sequence of DSL, DGL, or SEQ ID NOs: 12-28 and 30-58. In some embodiments, the antibody or antigen-binding fragment thereof, comprises three CDR sequences selected from any of DSL, DGL, or SEQ ID NOs: 12-28 and 30-58. In some embodiments, the antibody or antigen-binding fragment thereof, comprises six CDR sequences of DSL, DGL, or of SEQ ID NOs: 12-28 and 30-58. In some embodiments, the antibody or antigen-binding fragment thereof comprises three heavy chain CDR sequences selected from any of DSL, DGL, or SEQ ID NOs: 15-17, 21-28, 33-38, 42-50, and 54-58.

In some aspects, the present disclosure provides anti-EMR2 antibodies, or antigen-binding fragments thereof, comprising a CDR1, CDR2, and CDR3 from any of DSL, DGL, or SEQ ID NOs: 15-17, 21-28, 33-38, 42-50, and 54-58. In some aspects, the present disclosure provides anti-EMR2 antibodies, or antigen-binding fragments thereof, comprising at least one CDR that is at least 85%, 90%, 91%, 92%, 93%, 94%, 95%, 96%, 97%, 98%, 99%, or 100% identical to a CDR (e.g., CDR1, CDR2, and/or CDR3) selected from DSL, DGL, or SEQ ID NOs: 15-17, 21-28, 33-38, 42-50, and 54-58.

In some aspects, the present disclosure provides anti-EMR2 antibodies, or antigen-binding fragments thereof, comprising a heavy chain variable region comprising a CDR1 provided by any one of SEQ ID NO: 15, 21, 24, 27, 33, 36, 42, 45, 48, 54, and 57; a CDR2 provided by any one of SEQ ID NO: 16, 22, 25, 28, 34, 37, 43, 46, 49, 55, and 58; a CDR3 provided by any one of DSL, DGL, or SEQ ID NO: 17, 23, 26, 35, 38, 44, 47, 50, and 56; and a light chain variable region comprising a CDR1 provided by any one of SEQ ID NO: 12, 18, 30, 39, and 51; a CDR2 provided by any one of SEQ ID NO: 13, 19, 31, 40, and 52; and a CDR3 provided by any one of SEQ ID NO: 14, 20, 32, 41, and 53.

In some embodiments, the antibody or antigen-binding fragment thereof, comprises a heavy chain variable region (VH) that is at least 85%, 90%, 91%, 92%, 93%, 94%, 95%, 96%, 97%, 98%, 99%, or 100% identical to any one of SEQ ID NOs: 63, 69, 73, 75, 79, 83, 87, 91, 93, 97, and 101, and a light chain variable region (VL) that is at least 85%, 90%, 91%, 92%, 93%, 94%, 95%, 96%, 97%, 98%, 99%, or 100% identical to any one of SEQ ID NOs: 61, 67, 77, 85, and 95.

In some embodiments, the antibody or antigen-binding fragment thereof, comprises a heavy chain variable region (VH) set forth as any one of SEQ ID NOs: 63, 69, 73, 75, 79, 83, 87, 91, 93, 97, and 101, and a light chain variable region (VL) set forth as any one of SEQ ID NOs: 61, 67, 77, 85, and 95.

In some aspects, the present disclosure provides anti-EMR2 antibodies, or antigen-binding fragments thereof, comprising a VH comprising a CDR1 provided by any one of SEQ ID NO: 15, 21, 24, 27, 33, 36, 42, 45, 48, 54, and 57; a CDR2 provided by any one of SEQ ID NO: 16, 22, 25, 28, 34, 37, 43, 46, 49, 55, and 58; a CDR3 provided by any one of DSL, DGL, or of SEQ ID NO: 17, 23, 26, 35, 38, 44, 47, 50, and 56. In some embodiments, the antibody or antigen-binding fragment thereof, comprises a VH comprising an amino acid sequence of SEQ ID NOs: 63, 69, 73, 75, 79, 83, 87, 91, 93, 97, and 101.

In some aspects, the present disclosure provides anti-EMR2 antibodies, or antigen-binding fragments thereof, comprising a VH is at least 85%, 90%, 91%, 92%, 93%, 94%, 95%, 96%, 97%, 98%, 99%, or 100% identical to any one of SEQ ID NOs: 63, 69, 73, 75, 79, 83, 87, 91, 93, 97, and 101.

In some embodiments, the antibody or antigen-binding fragment thereof, wherein the antibody, or antigen-binding fragment thereof, is a monoclonal antibody, or antigen-binding fragment thereof. In some embodiments, the antibody or antigen-binding fragment thereof, is a human antibody or a humanized antibody, or antigen-binding fragment thereof.

In some aspects, the present disclosure provides anti-EMR2 antibodies, or antigen-binding fragments thereof, that compete with any of the antibodies, or antigen-binding fragments thereof.

In some embodiments, the antibody, or antigen-binding fragment thereof, comprises a CH1 constant domain, CH2 constant domain, and a CH3 constant domain. In some embodiments, the antibody, or antigen-binding fragment thereof, comprises an amino acid sequence of any one of SEQ ID NOs: 65, 71, 73, 75, 81, 83, 89, 91, 93, 99, and 101. In some embodiments, the antibody, or antigen-binding fragment thereof, further comprises a linker. In some embodiments, the antibody, or antigen-binding fragment thereof, the antibody is of the IgG1-, IgG2-, IgG3 or IgG4-type.

In some embodiments, the antibody, or antigen-binding fragment thereof, is a heavy chain antibody. In some embodiments, the antibody, or antigen-binding fragment thereof, is a camelid antibody. In some aspects, the present disclosure provides chimeric antigen receptors (CARs) comprising any of the antibodies, or antigen-binding fragment thereof, described herein.

In some aspects, the present disclosure provides a cell expressing any of the CARs described herein. In some embodiments, the cell is an immune effector cell. In some embodiments, the cell is a lymphocyte. In some embodiments, the cell is a T-cell. In some embodiments, the cell is a Natural Killer (NK) cell.

In some aspects, the present disclosure provides pharmaceutical compositions comprising any of the antibodies, or antigen-binding fragments thereof, described herein, any of the chimeric antigen receptors described herein, or any of the cells described herein, and a pharmaceutically acceptable excipient.

In some aspects, the present disclosure provides a nucleic acid comprising a nucleotide sequence encoding any of the antibodies, or antigen-binding fragments thereof, described herein, or any of the chimeric antigen receptors described herein. In some embodiments, the nucleic acid comprises a nucleotide sequence of any one of SEQ ID NOs: 64, 70, 72, 74, 80, 82, 88, 90, 92, 98, and 100.

In some aspects, the present disclosure provides vectors comprising any of the nucleic acids described herein. In some aspects, the present disclosure provides cells comprising any of the nucleic acids described herein or any of the vectors described herein. In some embodiments, the cell is an immune cell. In some embodiments, the immune cell is a T cell, a Natural Killer (NK) cell, a cytotoxic T lymphocyte (CTL), or a regulatory T cell.

In some aspects, the present disclosure provides methods of producing an antibody, or antigen-binding fragment thereof, comprising culturing any of the cells described herein under conditions suitable for expression of the antibody or antigen-binding fragment thereof.

In some aspects, the present disclosure provides methods of treating a disease or disorder associated with expression of EMR2 or a variant thereof, the method comprising administering to a subject in need thereof a therapeutically effective amount of an agent that targets EMR2, wherein the agent comprises any of the antibodies, or antigen-binding fragments thereof, described herein, any of the CARs described herein, or any of the cells described herein. In some embodiments, the disease or disorder associated with expression of EMR2 or a mutant variant thereof is a hematopoietic malignancy or a premalignancy. In some embodiments, the hematopoietic malignancy or premalignancy is acute myeloid leukemia (AML) or myelodysplastic syndrome (MDS). In some embodiments, further comprising administering to the subject an effective amount of a chemotherapeutic agent or an oncolytic therapeutic agent.

In some embodiments, the method further comprises administering a genetically engineered cell, or cell population thereof, comprising a genetic modification in a gene encoding EMR2 resulting in reduced or eliminated expression of EMR2 or expression of a variant of EMR2 as compared to EMR2 expressed by wild-type cells of the same cell type that do not harbor a genetic modification in the gene. In some embodiments, administration of the agent targeting EMR2 occurs simultaneously or in temporal proximity with administration of the genetically engineered cell, or cell population thereof. In some embodiments, administration of the agent targeting EMR2 occurs after administration of the genetically engineered cell, or cell population thereof. In some embodiments, administration of the agent targeting EMR2 occurs before administration of the genetically engineered cell, or cell population thereof.

In some embodiments, the genetically engineered cell is a stem cell. In some embodiments, the genetically engineered cell is a hematopoietic cell. In some embodiments, the genetically engineered cell is a hematopoietic stem cell. In some embodiments, the genetically engineered cell is a hematopoietic progenitor cell. In some embodiments, the genetically engineered cell is derived from the subject in need thereof. In some embodiments, the genetically engineered cell is obtained from the subject in need thereof. In some embodiments, the genetically engineered cell is obtained from an allogenic donor.

### BRIEF DESCRIPTION OF THE FIGURES

FIGS. 1A and 1B show histograms of EMR2-binding by the indicated EMR2 scFv and VH binders as measured by flow cytometry. FIG. 1A shows binding to the AML cell line MOLM13 (“MOLM13”), which expresses EMR2. FIG. 1B shows binding to HEK293 control cells (“293s”), which do not express EMR2. FITC-A fluorescence intensities are plotted against cell counts (y-axis, linear scale). Blank controls are shown as black histograms; EMR2 binders are shown as gray histograms.

FIGS. 2A and 2B show the lack of cross reactivity of nine out of ten tested EMR2 scFv and VH binders with CD97, as assessed by ELISA. FIG. 2A shows the optical density (OD) at 405 nM measured at the indicated concentrations of each of EMR2-10-scFv, EMR2-1-scFv, EMR2-2-scFv, EMR2-3VH, and EMR2-4-VH. FIG. 2B shows the OD405 at the indicated concentrations of each of EMR2-5-scFv, EMR2-6-VH, EMR2-7-scFv, EMR2-8-VH, and EMR2-9-VH.

FIGS. 3A and 3B show binding affinity of the indicated EMR2 scFv and VH binders as measured by ELISA. FIG. 3A shows a plot with binding to human EMR2 at the indicated concentrations of each of the EMR2-1-scFv, EMR2-2-scFv, EMR2-3-VH, and EMR2-4-VH. FIG. 3B shows a table with the half maximal binding affinity (EC50) values for each binder shown in FIG. 3A.

FIGS. 4A and 4B show binding affinity of the indicated EMR2 scFv and VH binders as measured by multipoint flow cytometric analysis. FIG. 4A shows a plot with binding of the EMR2 scFv and VH binders to MOLM13 cells, which express EMR2, at the indicated concentrations. The geometric mean fluorescent intensity (GMFI) was measured by flow cytometry. FIG. 4B shows a table with the half-maximal binding affinity (EC50) values for each binder shown in FIG. 4A.

FIG. 5A is a table showing a summary of the biochemical properties for each of the indicated EMR2 scFv and VH binders, including half-maximal binding affinity (EC50), dissociation constant (KD), and aggregation. NC=Not calculable. FIG. 5B shows flow cytometric analysis of EMR2-directed binder reactivity to different EMR2 isoforms and CD97 in 293T cells.

FIG. 6 shows an exemplary plasmid map encoding a chimeric antigen receptor (CAR) containing an EMR2 binder (e.g., EMR2-1-scFv), CD8 hinge region, CD8 transmembrane domain, 4-1BB costimulatory domain, and a CD3 intracellular signaling domain under control of a tEF-1a promoter.

FIG. 7 presents flow cytometric plots showing surface expression of CARs containing the indicated scFv binders in Jurkat cells on day 5 following transduction. The panels show CAR detection with a goat anti-human (H+L) antibody, and present untransduced cells (UTD), a control CAR construct containing an scFv binder (CAR2), and CAR constructs containing the indicated EMR2 binders: EMR2-10-scFv, EMR2-1-scFv, EMR2-2-scFv, EMR2-5-scFv, and EMR2-7-scFv.

FIG. 8 presents flow cytometric plots showing surface expression of CARs containing the indicated VH binders in Jurkat cells on day 5 following transduction. The panels show CAR detection with an anti-VH antibody and present untransduced cells (UTD), a control CAR containing a VH binder (CAR26), and CAR constructs containing the indicated EMR2 binders: EMR2-3-VH, EMR2-4-VH, and EMR2-6-VH.

FIG. 9 presents flow cytometric plots showing surface expression of CARs containing the indicated scFv binders in primary T cells. The panels show detection with an anti-human IgG (H+L) antibody and present untransduced cells (UTD) and CAR constructs containing the indicated EMR2 binders: EMR2-10-scFv, EMR2-1-scFv, EMR2-2-scFv, EMR2-5-scFv, and EMR2-7-scFv.

FIG. 10 presents flow cytometric plots showing surface expression of the CARs containing the indicated VH binders in primary T cells. The panels show detection with an anti-IgG VH domain antibody and presents untransduced cells (UTD) and CAR constructs containing the indicated EMR2 binders: EMR2-3-VH, EMR2-4-VH, and EMR2-6-VH.

FIGS. 11A and 11B show transduction efficiency of CAR constructs containing the indicated EMR2 scFv and VH binders. The transduction efficiency is presented as percentage of CAR+ cells compared to untransduced (UTD) cells. FIG. 11A shows transduction efficiency of Jurkat cells. FIG. 11B shows transduction efficiency of primary T cells.

FIGS. 12A and 12B show cell growth of cells expressing CAR constructs containing the indicated EMR2 scFv and VH binders. FIG. 12A shows growth of Jurkat cells expressing the indicated CARs. FIG. 12B shows growth of primary T cells expressing the indicated CARs. Total cell numbers are shown compared to untransduced (UTD) cells.

FIGS. 13A and 13B show the percentage of viable cells after transduction with CAR constructs containing the indicated EMR2 scFv and VH binders, compared to untransduced (UTD) control cells. FIG. 13A shows viability of Jurkat cells. FIG. 13B shows viability of primary T cells.

FIG. 14 shows a table presenting results from transduction of primary T cells with CAR constructs containing the indicated EMR2 scFv and VH binders. Cell viability, vector copy number (VCN), and transduction efficiency were tested for each of the indicated scFv and VH CARs compared to untransduced cells (UTD). Cell viability is expressed as a percentage of the control. VCN is the vector copy number in transduced cells. Transduction efficiency is represented as percentage of CAR+ cells compared to untransduced (UTD) cells.

FIGS. 15A-15D show results of generating EMR2-deficient cells. FIG. 15A shows of the percentage editing frequency on day 5 following transduction of cells with a Cas9 endonuclease and a gRNA comprising a spacer sequence of CUUGGCCAAUAACACCAUCC (SEQ ID NO: 104) (EMR2 gRNA). FIG. 15B shows the total number of reads and aligned reads. FIG. 15C shows surface expression of EMR2 presented as the percentage of EMR2+ cells in a wild-type (WT) population of cells or cells edited using a Cas endonuclease and the EMR2 specific gRNA described in FIG. 15A. FIG. 15D shows the total EMR2 geometric mean fluorescence intensity (gMFI) from live cells in a wild-type (WT) population of cells or cells edited using the EMR2 specific gRNA described in FIG. 15A.

FIGS. 16A-16D show EMR2-specific cytotoxic activity of EMR2-CAR-T cells. Primary T cells expressing the indicated EMR2-specific CAR constructs were co-cultured with CFSE labeled target cells, either MOLM13 wild-type (WT) or MOLM13 EMR2 knockout (EMR2KO) cells (n=3). FIG. 16A shows the percentage of viable target cells measured following 24 hour co-culture of EMR2-specific CARs with target cells at 1:1 ratio. FIG. 16B shows the percentage of viable target cells following 48 hour co-culture at 1:1 effector to target ratio. Percentage of viable target cells was determined by the absence of Annexin V and fixable viability dye reactivity by flow cytometry. FIG. 16C shows the percentage of CD25+ CD69+ T cells following 24 hour co-culture of the EMR2-specific CARs with CFSE labeled MOLM13 WT or MOLM13 EMR2KO cells. FIG. 16D shows the percentage of CD25+ CAR+ T cells following 48 hour co-culture.

FIGS. 17A-17I show the levels of cytokines produced by EMR2-CAR-T cells. Primary T cells expressing CAR constructs containing the indicated EMR2 binders were co-cultured with MOLM13 WT, MOLM13 EMR2 KO, or effectors alone, and co-culture supernatants were taken for cytokine analysis. FIG. 17A shows IL-2; FIG. 17B shows IFNγ; FIG. 17C shows TNFα; FIG. 17D shows Granzyme B at 24 hr; FIG. 17E shows Granzyme B at 48 hr; FIG. 17F shows sFasL at 24 hr; FIG. 17G shows IL-4 at 24 hr; FIG. 17H shows IL-5 at 24 hr; and FIG. 17I shows IL-13 at 24 hr. The supernatants of co-cultures were analyzed for cytokine secretion using a Luminex 17-Plex kit. Data shown as median±standard error of mean.

FIGS. 18A-18B show results obtained from analyzing the relationship between cell surface levels of EMR2 and EMR2 CAR-induced cytotoxicity. FIG. 18A shows data obtained from flow cytometry analysis of wild-type MOLM-13 cells (“Molm-13 WT”), MOLM-13 cells engineered using CRISPR/Cas9 to have reduced cell surface levels of EMR2 (“EMR2KO”), and MOLM-13 cells engineered using CRISPR/Cas9 to have reduced cell surface levels of EMR2 and express an exogenous copy of the EMR2 gene operably linked to a heterologous promoter (“Low” and “Very Low”). FIG. 18B shows reductions in cell viability of wild-type MOLM-13 cells (“Molm-13 WT”), MOLM-13 cells engineered using CRISPR/Cas9 to have reduced cell surface levels of EMR2 (“EMR2KO”), and MOLM-13 cells engineered using CRISPR/Cas9 to have reduced cell surface levels of EMR2 and express an exogenous copy of the EMR2 gene operably linked to a heterologous promoter (“EMR2 Low” and “EMR2 Very Low”) after co-culture with anti-EMR2 clone EMR2-5-scFv effector cells.

FIG. 19 shows flow cytometric analysis of EMR2 expression on healthy donor bone marrow and peripheral blood. Data shown as mean±standard deviation. The number of EMR2+ antigens are presented per cell for the indicated populations: hematopoietic stem cell (HSC), multipotent progenitor (MPP), common myeloid progenitor (CMP), granulocyte-monocyte progenitor (GMP), megakaryocytic-erythroid progenitor (MEP), common lymphoid progenitor (CLP), basophils, eosinophils, neutrophils, cDCs, pDCs, monocytes, and mast cells.

FIGS. 20A-20C show characterization of naturally occurring EMR2 genetic variants. FIG. 20A shows the structure of the EMR2 gene and indicates the locations of loss-of-function (LOF) homozygous variants. The number of individuals identified for homozygous LOF variants at the EMR2 gene regions indicated by “A”, “B”, “C”, “D”, “E”, “F”, “G”, and “H” is shown in the top right. FIG. 20B shows the presence and distribution of exon 6 only and exon 6-7 containing isoforms in samples of healthy bone marrow (BM) and hematopoietic stem and progenitor cells (HSPCs) as analyzed by ddPCR that amplified regions A-H indicated in FIG. 20A. FIG. 20C shows surface and total protein expression of EMR2 variants determined by flow cytometry and Western blotting.

FIGS. 21A-21C show analyses of EMR2-edited hematopoietic stem cells at16-week post-engraftment xenotransplantation into NOD-scid IL2Rg null (NSG) mice. FIG. 21A shows flow cytometry analysis of bone marrow (BM) indicating comparable levels of total human leukocyte chimerism, human HSPCs and various human hematopoietic lineages, between mice engrafted with control cells (CTR) or edited cells (EMR2). FIG. 21B shows a significant reduction the EMR2 protein expression in total human cells was observed in the bone marrow. FIG. 21C shows quantification of on-target editing by Next-Gen Sequencing (NGS) of PCR amplicons amplified from the EMR2 locus. The editing frequency of bone marrow (BM) samples from each individual mouse are plotted along with the input samples used to engraft the mice, indicated as “Input.” N=10 mice per group. Data shown as mean±standard deviation.**** p<0.0001 compared to CTR by Unpaired t test.

### DETAILED DESCRIPTION

The present disclosure is based, in part, on the discovery of novel agents that selectively bind to EMR2. In some embodiments, the agents are antibodies that selectively bind to EMR2. In some embodiments, the antibodies comprise a heavy chain variable domain. In some embodiments, the antibodies are single-domain antibodies. In some embodiments, the antibodies comprise a heavy chain variable domain and a light chain variable domain. In some embodiments, the antibodies comprise a heavy chain variable domain and one or more constant domains. The present disclosure also describes chimeric antigen receptors (CARs) that selectively bind to EMR2. The disclosure also relates to nucleic acids encoding said antibodies or chimeric antigen receptors, methods of producing said antibodies or chimeric antigen receptors, and methods of use in the treatment of treat malignancies using the same (e.g., acute myeloid leukemia (AML), myelodysplastic syndrome (MDS)).

### Antibodies

The term “antibody” is used herein in the broadest sense and encompasses various antibody structures, including but not limited to monoclonal antibodies, polyclonal antibodies, multispecific antibodies (e.g., bispecific antibodies), and/or antibody fragments (preferably those fragments that exhibit the desired antigen-binding activity). An antibody described herein can be an immunoglobulin, heavy chain antibody, light chain antibody, LRR-based antibody, or other protein scaffold with antibody-like properties, as well as other immunological binding moiety known in the art, including, e.g., a Fab, Fab′, Fab′2, Fab2, Fab3, F(ab′)2, Fd, Fv, Feb, scFv, SMIP, diabody, triabody, tetrabody, minibody, Nanobody® (single domain antibody), maxibody, tandab, DVD, BiTe, TandAb, or the like, or any combination thereof. In some embodiments, the antibody is a heavy chain antibody. In some embodiments, the antibody is a camelid antibody. In some embodiments, the antibody is a llama antibody. In some embodiments, the antibody is a human antibody. In some embodiments, the antibody is a naive human antibody. In some embodiments, the antibody comprises a heavy chain variable region and one or more constant regions (e.g., CH2 and CH3). In some embodiments, the antibody is a Nanobody®, also referred to as a single domain antibody or “VHH.” The subunit structures and three-dimensional configurations of different classes of antibodies are known in the art.

A “monoclonal antibody” or “mAb” refers to an antibody obtained from a population of substantially homogeneous antibodies, i.e., the individual antibodies comprising the population are identical and/or bind the same epitope, except for possible variant antibodies (e.g., containing naturally occurring mutations or arising during production of a monoclonal antibody preparation), such variants generally being present in minor amounts. In contrast to polyclonal antibody preparations, which typically include different antibodies directed against different determinants (epitopes), each monoclonal antibody of a monoclonal antibody preparation is directed against a single determinant on an antigen.

An “antigen-binding fragment” refers to a portion of an intact antibody that binds the antigen to which the intact antibody binds. An antigen-binding fragment of an antibody includes any naturally occurring, enzymatically obtainable, synthetic, or genetically engineered polypeptide or glycoprotein that specifically binds an antigen to form a complex. Exemplary antibody fragments include, but are not limited to, Fv, Fab, Fab′, Fab′-SH, F(ab′)2; diabodies; linear antibodies; single-chain antibody molecules (e.g., scFv), single-domain antibody molecules (e.g., VHH or VH or VL domains only); and multispecific antibodies formed from antibody fragments. In some embodiments, the antigen-binding fragments of the antibodies described herein are scFvs. In some embodiments, the antigen-binding fragments of the antibodies described herein are VH domains only. As with full antibody molecules, antigen-binding fragments may be mono-specific or multispecific (e.g., bispecific). A multispecific antigen-binding fragment of an antibody may comprise at least two different variable domains, wherein each variable domain is capable of specifically binding to a separate antigen or to a different epitope of the same antigen.

A “multispecific antibody” refers to an antibody comprising at least two different antigen binding domains that recognize and specifically bind to at least two different antigens. A “bispecific antibody” is a type of multispecific antibody and refers to an antibody comprising two different antigen binding domains that recognize and specifically bind to at least two different antigens.

A “different antigen” may refer to different and/or distinct proteins, polypeptides, or molecules; as well as different and/or distinct epitopes, which epitopes may be contained within one protein, polypeptide, or other molecule.

The term “epitope” refers to an antigenic determinant that interacts with a specific antigen binding site in the variable region of an antibody molecule known as a paratope. A single antigen may have more than one epitope. Thus, different antibodies may bind to different areas of an antigen and may have different biological effects. The term “epitope” also refers to a site of an antigen to which B and/or T cells respond. It also refers to a region of an antigen that is bound by an antibody. Epitopes may be defined as structural or functional. Functional epitopes are generally a subset of the structural epitopes and have those residues that directly contribute to the affinity of the interaction. Epitopes may also be conformational, that is, composed of non-linear amino acids. In certain embodiments, epitopes may include determinants that are chemically active surface groupings of molecules such as amino acids, sugar side chains, phosphoryl groups, or sulfonyl groups, and, in certain embodiments, may have specific three-dimensional structural characteristics, and/or specific charge characteristics.

As used herein, “selective binding,” “selectively binds,” “specific binding,” or “specifically binds” refers, with respect to an antigen binding moiety and an antigen target, preferential association of an antigen binding moiety to an antigen target and not to an entity that is not the antigen target. A certain degree of non-specific binding may occur between an antigen binding moiety and a non-target. In some embodiments, an antigen binding moiety selectively binds an antigen target if binding between the antigen binding moiety and the antigen target is greater than 2-fold, greater than 5-fold, greater than 10-fold, or greater than 100-fold as compared with binding of the antigen binding moiety and a non-target. In some embodiments, an antigen binding moiety selectively binds an antigen target if the binding affinity is less than about 10⁻⁵ M, less than about 10⁻⁶ M, less than about 10⁻⁷ M, less than about 10⁻⁸ M, or less than about 10⁻⁹ M. In some embodiments, an antigen binding moiety selectively binds an epitope of an antigen target if binding between the antigen binding moiety and the epitope of the antigen target is greater than 2-fold, greater than 5-fold, greater than 10-fold, or greater than 100-fold as compared with binding of the antigen binding moiety and a non-target or another epitope of the antigen target. In some embodiments, an antigen binding moiety selectively binds an epitope of an antigen target if the binding affinity is less than about 10⁻⁵ M, less than about 10⁻⁶ M, less than about 10⁻⁷ M, less than about 10⁻⁸ M, or less than about 10⁻⁹ M.

In some embodiments, antibodies or fragments thereof that selectively bind to an identical epitope or overlapping epitope that will often cross-compete for binding to an antigen. Thus, in some embodiments, the disclosure provides an antibody or fragment thereof that cross-competes with an exemplary antibody or fragment thereof as disclosed herein. In some embodiments, to “cross-compete,” “compete,” “cross-competition,” or “competition” means antibodies or fragments thereof compete for the same epitope or binding site on a target. Such competition can be determined by an assay in which the reference antibody or fragment thereof prevents or inhibits specific binding of a test antibody or fragment thereof, and vice versa. Numerous types of competitive binding assays can be used to determine if a test molecule competes with a reference molecule for binding. Examples of assays that can be employed include solid phase direct or indirect radioimmunoassay (RIA), solid phase direct or indirect enzyme immunoassay (EIA), sandwich competition assay (see, e.g., Stahli et al. Methods in Enzymology (1983) 9:242-253), solid phase direct biotin-avidin EIA (see, e.g., Kirkland et al., J. Immunol. (1986) 137:3614-9), solid phase direct labeled assay, solid phase direct labeled sandwich assay, Luminex (Jia et al. “A novel method of Multiplexed Competitive Antibody Binning for the characterization of monoclonal antibodies” J. Immunological Methods (2004) 288, 91-98), and surface plasmon resonance (Song et al. “Epitope Mapping of Ibalizumab, a Humanized Anti-CD4 Monoclonal Antibody with Anti-HIV-1 Activity in Infected Patients” J. Virol. (2010) 84, 6935-42). In some embodiments, when a competing antibody or fragment thereof is present in excess, it will inhibit binding of a reference antibody or fragment thereof to a common antigen by at least 50%, 55%, 60%, 65%, 70%, or 75%. In some instances, binding is inhibited by at least 80%, 85%, 90%, 95%, 96%, 97%, 98%, 99%, or more.

An antibody can be an immunoglobulin molecule of four polypeptide chains, e.g., two heavy (H) chains and two light (L) chains. In some embodiments, a light chain is a lambda light chain. In some embodiments, a light chain is a kappa light chain. A heavy chain can include a heavy chain variable domain and a heavy chain constant domain. A heavy chain constant domain can include, any one or more of a CH1, hinge, CH2, CH3, and in some instances CH4 regions. A light chain can include a light chain variable domain and a light chain constant domain. A light chain constant domain can include a CL.

A heavy chain variable domain of a heavy chain and a light chain variable domain of a light chain can typically be further subdivided into regions of variability, termed complementarity determining regions (CDRs), interspersed with regions that are more conserved, termed framework regions (FR). In some embodiments, such heavy chain and/or light chain variable domains can each include three CDRs and four framework regions, arranged from amino-terminus to carboxyl-terminus in the following order: FR1, CDR1, FR2, CDR2, FR3, CDR3, FR4, one or more of which can be engineered as described herein. The CDRs in a heavy chain are designated “CDRH1,” “CDRH2,” and “CDRH3,” respectively, and the CDRs in a light chain are designated “CDRL1,” “CDRL2,” and “CDRL3.”

There are five major classes of antibodies: IgA, IgD, IgE, IgG, and IgM, and several of these may be further divided into subclasses (isotypes), e.g., IgG1, IgG2, IgG3, IgG4, IgA1, and IgA2. The heavy chain constant domains that correspond to the different classes of immunoglobulins are called α, δ, ε, γ, and μ, respectively.

### Exemplary Anti-EMR2 Antibodies

Provided herein are anti-EMR2 antibodies, and antigen-binding fragments thereof that bind selectively to EMR2. In some embodiments, the anti-EMR2 antibodies described herein are single domain antibodies or single chain antibodies.

In some embodiments, the antibodies are single domain antibodies. Single domain antibodies are antibodies whose complementary determining regions are part of a single domain polypeptide. Examples include, but are not limited to, heavy chain antibodies, antibodies naturally devoid of light chains, single domain antibodies derived from conventional 4-chain antibodies, engineered antibodies and single domain scaffolds other than those derived from antibodies. Single domain antibodies may be derived from any species including, but not limited to mouse, human, camel, llama, goat, rabbit, and bovine. According to one aspect of the disclosure, a single domain antibody as used herein is a naturally occurring single domain antibody known as heavy chain antibody devoid of light chains. Such single domain antibodies are disclosed in, e.g., PCT Publication No. WO 94/04678. Such variable domains derived from a heavy chain antibody naturally devoid of light chain is referred to herein as a “VHH” or “Nanobody®”. Such a VHH can be derived from antibodies raised in Camelidae species, for example in camel, dromedary, llama, vicuna, alpaca and guanaco. Other species besides Camelidae may produce heavy chain antibodies naturally devoid of light chain; such VHHs are within the scope of the disclosure. In some embodiments, the antibody is a Nanobody® or “VHH” and comprises a heavy chain variable region. In some embodiments, the antibody comprises a heavy chain variable region and one or more heavy chain constant regions. In some embodiments, the antibody comprises a heavy chain variable region and does not comprise one or more heavy chain constant regions. In some embodiments, the antibody comprises a heavy chain variable region and does not comprise a light chain region (light chain variable region or light chain constant region).

The amino acid residues of VHH domains from Camelids are numbered according to the general numbering for VH domains given by Kabat et al., “Sequence of proteins of immunological interest,” US Public Health Services, NIH (Bethesda, MD), Publication No 91-3242 (1991); see also Riechmann et al., J. Immunol. Methods (1999) 231:25-38. According to this numbering, FR1 comprises the amino acid residues at positions 1-30, CDR1 comprises the amino acid residues at positions 31-35, FR2 comprises the amino acids at positions 36-49, CDR2 comprises the amino acid residues at positions 50-65, FR3 comprises the amino acid residues at positions 66-94, CDR3 comprises the amino acid residues at positions 95-102, and FR4 comprises the amino acid residues at positions 103-113.

It should be noted, however (as is well known in the art for VH domains and for VHH domains), that the total number of amino acid residues in each of the CDRs may vary and may not correspond to the total number of amino acid residues indicated by the Kabat numbering (that is, one or more positions according to the Kabat numbering may not be occupied in an actual sequence, or the actual sequence may contain more amino acid residues than the number allowed for by the Kabat numbering). This means that, generally, the numbering according to Kabat may or may not correspond to the actual numbering of the amino acid residues in an actual sequence.

Alternative methods for numbering the amino acid residues of VH domains, which methods can also be applied in an analogous manner to VHH domains, are known in the art. However, in the present disclosure the numbering according to Kabat and applied to VHH domains as described above will be followed, unless indicated otherwise.

In some embodiments, the position numbering of an amino acid residue may be referred to based on a corresponding amino acid residue in a reference sequence.

The present disclosure provides antibodies that can include various heavy chains described herein. In some embodiments, an antibody comprises two heavy chains and light chains. In some embodiments, an antibody comprises two heavy chains, which may be two of the same heavy chain (having the same amino acid sequence) or different heavy chain (having a different amino acid sequence). In some embodiments, an antibody comprises two heavy chains, which may bind to the same epitope or a different epitope of a target antigen. In some embodiments, an antibody comprises two heavy chains, which may bind to epitopes of different target antigens. In some embodiments, the present disclosure encompasses an antibody including at least one heavy chain as disclosed herein, at least one heavy chain framework domain as disclosed herein, and/or at least one heavy chain CDR sequence as disclosed herein.

In some embodiments, an antibody disclosed herein is a homodimeric monoclonal antibody. In some embodiments, an antibody disclosed herein is a heterodimeric antibody. In some embodiments, an antibody is, e.g., a typical antibody or a diabody, triabody, tetrabody, minibody, Nanobody® (single domain antibody), maxibody, tandab, DVD, BiTe, scFv, TandAb scFv, Fab, Fab2, Fab3, F(ab′)2, or the like, or any combination thereof. In some embodiments, the antibody is a heavy chain antibody. In some embodiments, the antibody is a camelid antibody. In some embodiments, the antibody is a llama antibody. In some embodiments, the antibody comprises a heavy chain variable region and one or more constant regions. In some embodiments, the antibody is a Nanobody®, also referred to as a single domain antibody or “VHH.” In some embodiments, the antibody comprises one, two, or three immunoglobulin constant domains (e.g., chosen from CH1, CH2, CH3, and CH4). In some embodiments, the antibody comprises one, two, or three IgG1 constant domains. In some embodiments, the antibody comprises a CH2 and a CH3 domain. In some embodiments, the antibody comprises a CH fusion. An exemplary IgG1 CH2 and CH3 domain for use in an antibody of the disclosure is provided below:

(SEQ ID NO: 11)
APELLGGPSVFLFPPKPKDTLMISRTPEVTCVVVDVSHEDPEVKFNWYV
DGVEVHNAKTKPREEQYNSTYRVVSVLTVLHQDWLNGKEYKCKVSNKAL
PAPIEKTISKAKGQPREPQVYTLPPSRDELTKNQVSLTCLVKGFYPSDI
IAVEWESNGQPENNYKTTPPVLDSDGSFFLYSKLTVDKSRWQQGNVFSC
SVMHEALHNHYTQKSLSLSPGK

In some embodiments, the antibodies are single chain antibodies.

In some embodiments, the anti-EMR2 antibody, or antigen-binding fragment thereof, comprises an amino acid sequence of any one of SEQ ID NOs: 61, 63, 67, 69, 73, 75, 77, 79, 83, 85, 87, 91, 93, 95, 97, and 101.

In some embodiments, the anti-EMR2 antibody, or antigen-binding fragment thereof, comprises an amino acid sequence of any of SEQ ID NOs: 65, 71, 73, 75, 81, 83, 89, 91, 93, 99, or 101.

In some embodiments, the anti-EMR2 antibody, or antigen-binding fragment thereof, comprises a CDR sequence encompassed within any one of SEQ ID NOs: 61, 63, 67, 69, 73, 77, 79, 83, 85, 87, 91, 93, 95, 97, and 101.

In some embodiments, the anti-EMR2 antibody, or antigen-binding fragment thereof, comprises a CDR comprising the sequence of any one of DSL, DGL, or SEQ ID NOs: 12-28 and 30-58.

In some embodiments, the anti-EMR2 antibody, or antigen-binding fragment thereof, comprises a heavy chain CDR1, CDR2, and CDR3 encompassed within any one of SEQ ID NOs: 63, 69, 73, 75, 79, 83, 87, 91, 93, 97, and 101. In some embodiments, the anti-EMR2 antibody, or antigen-binding fragment thereof, comprises a light chain CDR1, CDR2, and CDR3 encompassed within any one of SEQ ID NOs: 61, 67, 77, 85, and 95.

In some embodiments, the anti-EMR2 antibody, or antigen-binding fragment thereof, comprises a heavy chain CDR1, CDR2, and CDR3 of any one of DSL, DGL, or SEQ ID NOs: 15-17, 21-23, 24-26, 27-28, 33-35, 36-38, 42-50, or 54-58.

In some embodiments, the anti-EMR2 antibody, or antigen-binding fragment thereof, comprises at least one CDR (e.g., CDR1, CDR2, and/or CDR3) of DSL, DGL, or SEQ ID NOs: 12-28 and 30-58.

In some embodiments, the anti-EMR2 antibody, or antigen-binding fragment thereof, comprises two CDRs (e.g., CDR1, CDR2, and/or CDR3) of any one of DSL, DGL, or SEQ ID NOs: 12-28 and 30-58.

In some embodiments, the anti-EMR2 antibody, or antigen-binding fragment thereof, comprises three CDRs (e.g., CDR1, CDR2, and/or CDR3) of any one of DSL, DGL, or SEQ ID NOs: 12-28 and 30-58.

In some embodiments, the anti-EMR2 antibody, or antigen-binding fragment thereof, comprises four CDRs (e.g., CDR1, CDR2, and/or CDR3) of any one of DSL, DGL, or SEQ ID NOs: 12-28 and 30-58.

In some embodiments, the anti-EMR2 antibody, or antigen-binding fragment thereof, comprises five CDRs (e.g., CDR1, CDR2, and/or CDR3) of any one of DSL, DGL, or SEQ ID NOs: 12-28 and 30-58.

In some embodiments, the anti-EMR2 antibody, or antigen-binding fragment thereof, comprises six CDRs (e.g., CDR1, CDR2, and/or CDR3) of any one of DSL, DGL, or SEQ ID NOs: 12-28 and 30-58.

In some embodiments, the anti-EMR2 antibody, or antigen-binding fragment thereof, comprises at least one CDR that is at least 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9% or 100% identical to a CDR (e.g., CDR1, CDR2, and/or CDR3) of any one of DSL, DGL, or SEQ ID NOs: 12-28 and 30-58.

In some embodiments, the anti-EMR2 antibody, or antigen-binding fragment thereof, comprises at least one CDR (e.g., CDR1, CDR2, and/or CDR3) depicted in any one of SEQ ID NOs: 65, 71, 73, 75, 81, 83, 89, 91, 93, 99, and 101.

In some embodiments, the anti-EMR2 antibody, or antigen-binding fragment thereof, comprises at least one CDR that is at least 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9% or 100% identical to a CDR (e.g., CDR1, CDR2, and/or CDR3) of any one of SEQ ID NOs: 65, 71, 73, 75, 81, 83, 89, 91, 93, 99, and 101.

In some embodiments, the anti-EMR2 antibody, or antigen-binding fragment thereof, comprises at least one CDR having one or more (e.g., 1, 2, 3, 4, 5, or more) additions, deletions, or substitutions relative to any one of the CDRs (e.g., CDR1, CDR2, and/or CDR3) provided by any one of DSL, DGL, or SEQ ID NOs: 12-28 and 30-58.

The present disclosure provides, among other things, an anti-EMR2 antibody, or antigen-binding fragment thereof, comprising a VH. In some embodiments, the anti-EMR2 antibody, or antigen-binding fragment thereof, comprises a VH comprising an amino acid sequence of any one of SEQ ID NOs: 63, 69, 73, 75, 79, 83, 87, 91, 93, 97, or 101. In some embodiments, the anti-EMR2 antibody, or antigen-binding fragment thereof, comprises a VH comprising a CDR sequence provided by any one of DSL, DGL, or SEQ ID NOs: 15-17, 21-28, 33-38, 42-50, or 54-58. In some embodiments, the anti-EMR2 antibody, or antigen-binding fragment thereof, comprises a VH comprising CDR1, CDR2, and CDR3 encompassed within DSL, DGL, or any one of SEQ ID NOs: 15-17, 21-28, 33-38, 42-50, or 54-58. In some embodiments, the anti-EMR2 antibody, or antigen-binding fragment thereof, comprises a VH comprising at least one CDR (e.g., CDR1, CDR2, and/or CDR3) provided by any one of DSL, DGL, or SEQ ID NOs: 15-17, 21-28, 33-38, 42-50, or 54-58. In some embodiments, the anti-EMR2 antibody, or antigen-binding fragment thereof, comprises a VH comprising at least one CDR that is at least 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100% identical to a CDR (e.g., CDR1, CDR2, and/or CDR3) provided by any one of DSL, DGL, or SEQ ID NOs: 15-17, 21-28, 33-38, 42-50, or 54-58. In some embodiments, the anti-EMR2 antibody, or antigen-binding fragment thereof, comprises a VH comprising at least one CDR having one or more (e.g., 1, 2, 3, 4, 5, or more) additions, deletions, or substitutions relative to any one of the CDRs (e.g., CDR1, CDR2, and/or CDR3) provided by DSL, DGL, or SEQ ID NOs: 15-17, 21-28, 33-38, 42-50, or 54-58.

In some embodiments, the anti-EMR2 antibody, or antigen-binding fragment thereof, is a monoclonal antibody, or antigen-binding fragment thereof. In some embodiments, an anti-EMR2 antibody, or antigen-binding fragment thereof, is a humanized antibody, or antigen-binding fragment thereof. In some embodiments, the anti-EMR2 antibody, or antigen-binding fragment thereof, is a camelid antibody or has been derived from a camelid antibody.

In some embodiments, the present disclosure provides an anti-EMR2 antibody, or antigen-binding fragment thereof, that competes with an antibody, or antigen-binding fragment thereof, comprising an amino acid sequence of SEQ ID NO: 61, 63, 67, 69, 73, 77, 79, 83, 85, 87, 91, 93, 95, 97, or 101. In some embodiments, the present disclosure provides an anti-EMR2 antibody, or antigen-binding fragment thereof, that competes with an antibody, or antigen-binding fragment thereof, comprising an amino acid sequence of SEQ ID NOs: 65, 71, 73, 75, 81, 83, 89, 91, 93, 99, or 101.

In some embodiments, the present disclosure provides an anti-EMR2 antibody, or antigen-binding fragment thereof, comprising between 1 and 24 (e.g., 1, 2, 3, 4, 5, 10, or more) additions, deletions, or substitutions relative to an anti-EMR2 antibody, or antigen-binding fragment thereof, wherein the anti-EMR2 antibody comprises an amino acid sequence of any one of SEQ ID NO: 61, 63, 67, 69, 73, 75, 77, 79, 83, 85, 87, 91, 93, 95, 97, or 101, and, e.g., the antibody or fragment selectively binds EMR2. In some embodiments, the present disclosure provides an anti-EMR2 antibody, or antigen-binding fragment thereof, comprising an amino acid sequence having at least 80%, 85%, 90%, 91%, 92%, 93%, 94%, 95%, 96%, 97%, 98%, or 99% sequence identity to an amino acid sequence of SEQ ID NOs: 65, 71, 73, 75, 81, 83, 89, 91, 93, 99, or 101 and, e.g., the antibody or fragment selectively binds EMR2.

In some embodiments, the present disclosure provides an anti-EMR2 antibody, or antigen-binding fragment thereof, comprises a heavy chain CDR1 provided by any one of SEQ ID NOs: 21, 24, 27, 33, 36, 42, 45, 48, 54, and 57, a heavy chain CDR2 provided by any one of SEQ ID NOs: 16, 22, 25, 28, 34, 37, 43, 46, 49, 55, and 58, and a heavy chain CDR3 provided by any one of DSL, DGL, or SEQ ID NOs: 17, 23, 26, 35, 38, 44, 47, 50, and 56. In some embodiments, the present disclosure provides an anti-EMR2 antibody, or antigen-binding fragment thereof, comprises: a light chain CDR1 provided by any one of SEQ ID NOs: 12, 18, 30, 39, and 51; a light chain CDR2 provided by any one of SEQ ID NOs: 13, 19, 31, 40, and 52; and a light chain CDR3 provided by any one of SEQ ID NOs: 14, 20, 32, 41, and 53.

In some embodiments, the present disclosure provides nucleic acids encoding any of the anti-EMR2 antibodies, or antigen-binding fragments thereof, described herein. In some embodiments, the present disclosure provides nucleic acids encoding any of the anti-EMR2 antibodies, or antigen-binding fragments thereof, comprising between 1 and 24 (e.g., 1, 2, 3, 4, 5, 10, or more) additions, deletions, or substitutions relative to an anti-EMR2 antibody, or antigen-binding fragment thereof, wherein the anti-EMR2 antibody comprises an amino acid sequence of SEQ ID NO: 61, 63, 67, 69, 73, 75, 77, 79, 83, 85, 87, 91, 93, 95, 97, or 101, and, e.g., the antibody or fragment selectively binds EMR2. In some embodiments, the present disclosure provides nucleic acids encoding any of the anti-EMR2 antibodies, or antigen-binding fragments thereof, comprising between 1 and 24 (e.g., 1, 2, 3, 4, 5, 10, or more) additions, deletions, or substitutions relative to an anti-EMR2 antibody, or antigen-binding fragment thereof, wherein the anti-EMR2 antibody comprises an amino acid sequence of SEQ ID NO: 65, 71, 73, 75, 81, 83, 89, 91, 93, 99, or 101 and, e.g., the antibody or fragment selectively binds EMR2. In some embodiments, the present disclosure provides nucleic acids encoding any of the anti-EMR2 antibodies, or antigen-binding fragments thereof, comprising an amino acid sequence having at least 80%, 85%, 90%, 91%, 92%, 93%, 94%, 95%, 96%, 97%, 98%, or 99% sequence identity to an amino acid sequence of DSL, DGL, or of any one of SEQ ID NOs: 12, 13, 14, 15, 16, 17, 18, 19, 20, 21, 22, 23, 24, 25, 26, 27, 28, 30, 31, 32, 33, 34, 35, 36, 37, 38, 39, 40, 41, 42, 43, 44, 45, 46, 47, 48, 49, 50, 51, 52, 53, 54, 55, 56, 57, 58, 61, 63, 65, 67, 69, 71, 75, 77, 79, 81, 83, 85, 87, 89, 91, 93, 95, 97, 99, and 101, e.g., the antibody or fragment selectively binds EMR2.

In some embodiments, the present disclosure provides a nucleic acid having any one of the sequences provided by any one of SEQ ID NOs: 64, 70, 72, 74, 80, 82, 88, 90, 92, 98, and 100 and encodes an antibody or antigen binding fragment thereof that binds EMR2, such as an antibody or antigen binding fragment comprising the sequence of DSL, DGL, or of any one of SEQ ID NOs: 12, 13, 14, 15, 16, 17, 18, 19, 20, 21, 22, 23, 24, 25, 26, 27, 28, 30, 31, 32, 33, 34, 35, 36, 37, 38, 39, 40, 41, 42, 43, 44, 45, 46, 47, 48, 49, 50, 51, 52, 53, 54, 55, 56, 57, 58, 61, 63, 65, 67, 69, 71, 75, 77, 79, 81, 83, 85, 87, 89, 91, 93, 95, 97, 99, and 101. In some embodiments, the present disclosure provides a nucleic acid having at least 80%, 85%, 90%, 91%, 92%, 93%, 94%, 95%, 96%, 97%, 98%, or 99% sequence identity to any one of the sequences provided by any one of SEQ ID NOs: 64, 70, 72, 74, 80, 82, 88, 90, 92, 98, and 100 and encodes an antibody or antigen binding fragment thereof that binds EMR2, such as an antibody or antigen binding fragment comprising the sequence of DSL, DGL, or of any one of SEQ ID NOs: 12, 13, 14, 15, 16, 17, 18, 19, 20, 21, 22, 23, 24, 25, 26, 27, 28, 30, 31, 32, 33, 34, 35, 36, 37, 38, 39, 40, 41, 42, 43, 44, 45, 46, 47, 48, 49, 50, 51, 52, 53, 54, 55, 56, 57, 58, 61, 63, 65, 67, 69, 71, 75, 77, 79, 81, 83, 85, 87, 89, 91, 93, 95, 97, 99, and 101.

The present disclosure provides, among other things, methods of making an anti-EMR2 antibody, or antigen-binding fragment thereof. Methods of making antibodies are known in the art. For example, monoclonal antibodies can be produced using a variety of known techniques, such as the standard somatic cell hybridization technique described, for example, by Kohler and Milstein, Nature (1975) 256: 495. Other techniques for producing monoclonal antibodies also can be employed, e.g., viral or oncogenic transformation of B lymphocytes or phage display technique using libraries of human antibody genes.

In some embodiments, human antibodies are obtained by cloning the heavy and light chain genes directly from human B cells obtained from a human subject. The B cells are separated from peripheral blood (e.g., by flow cytometry, e.g., FACS), stained for B cell marker(s), and assessed for antigen binding. The RNA encoding the heavy and light chain variable regions (or the entire heavy and light chains) is extracted and reverse transcribed into DNA, from which the antibody genes are amplified (e.g., by PCR) and sequenced. The known antibody sequences can then be used to express recombinant human antibodies against a known target antigen. In some instances, human antibodies may be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extra-chromosomally or integrated randomly into the animal's chromosomes. In such transgenic mice, the endogenous immunoglobulin loci have generally been inactivated. Human variable regions from intact antibodies generated by such animals may be further modified, for example, by combining with a different human constant region.

In some instances, antibodies can also be made by hybridoma-based methods. In some embodiments, an animal system for generating hybridomas which produce human monoclonal antibodies is the murine system. Hybridoma production in the mouse is well known in the art, including immunization protocols and techniques for isolating and fusing immunized splenocytes. Human myeloma and mouse-human heteromyeloma cell lines for the production of human monoclonal antibodies have been described.

Human antibodies may also be generated by isolating Fv clone variable domain sequences selected from human-derived phage display libraries. Such variable domain sequences may then be combined with a desired human constant domain.

In some embodiments, the present disclosure provides methods of producing an antibody, or antigen-binding fragment thereof, comprising culturing a host cell comprising a nucleic acid encoding any of the anti-EMR2 antibodies described herein. In some embodiments, the present disclosure provides methods of producing an antibody, or antigen-binding fragment thereof, comprising contacting a host cell with a nucleic acid comprising a nucleic acid encoding any of the anti-EMR2 antibodies described herein. In some embodiments, contacting the cell comprises introducing the nucleic acid into the host cell using electroporation, transfection, or transduction with a recombinant virus (e.g., a recombinant retrovirus, adenovirus, adeno-associated virus, lentivirus, and herpes simplex virus-1 (HSV-1)) comprising the nucleic acid. In some embodiments, the methods involve culturing a cell comprising a nucleic acid sequence of SEQ ID NOs: 64, 70, 72, 74, 80, 82, 88, 90, 92, 98, or 100 under conditions suitable for expression of the antibody or antigen-binding fragment thereof. In some embodiments, the methods further comprise collecting, isolating, and/or purifying the antibodies or antigen-binding fragments thereof.

Sequences of exemplary antibody clones, including exemplary single chain and single domain anti-EMR2 antibodies, are provided in Tables 1-11. In some embodiments, an antibody or antigen-binding fragment thereof, comprises an amino acid sequence having a least 75% sequence identity (e.g., 75-80%, 80-85%, 85-90%, 90-95%, or 95-99% sequence identity) to an amino acid sequence set forth in any one of Tables 1-11 (e.g., one or more of the sequences comprising DSL, DGL, or set forth in SEQ ID NOs: 12, 13, 14, 15, 16, 17, 18, 19, 20, 21, 22, 23, 24, 25, 26, 27, 28, 30, 31, 32, 33, 34, 35, 36, 37, 38, 39, 40, 41, 42, 43, 44, 45, 46, 47, 48, 49, 50, 51, 52, 53, 54, 55, 56, 57, 58, 61, 63, 65, 67, 69, 71, 75, 77, 79, 81, 83, 85, 87, 89, 91, 93, 95, 97, 99, and 101). In some embodiments, an amino acid sequence having at least 75% sequence identity to an amino acid sequence set forth in any one of Tables 1-11 comprises an amino acid substitution, an amino acid deletion, or an amino acid addition at one or more corresponding amino acid positions in DSL, DGL, or SEQ ID NO: 12, 13, 14, 15, 16, 17, 18, 19, 20, 21, 22, 23, 24, 25, 26, 27, 28, 30, 31, 32, 33, 34, 35, 36, 37, 38, 39, 40, 41, 42, 43, 44, 45, 46, 47, 48, 49, 50, 51, 52, 53, 54, 55, 56, 57, 58, 61, 63, 65, 67, 69, 71, 75, 77, 79, 81, 83, 85, 87, 89, 91, 93, 95, 97, 99, or 101. In some embodiments, the substitution replaces a residue at a corresponding amino acid position with a residue having a side chain of a similar charge and/or shape (e.g., a first hydrophilic amino acid residue replaced with a second hydrophilic amino acid residue or a first hydrophobic amino acid residue replaced with a second hydrophobic amino acid residue, such as a conservative substitution including, but not limited to, replacement of a glycine for an alanine, replacement of a glutamate for an aspartate, replacement of a lysine for an arginine, replacement of a leucine for an isoleucine, replacement of a phenylalanine for a tryptophan, replacement of an asparagine for a glutamine, replacement of a serine for a threonine, etc.). In some embodiments, an antibody or antigen-binding fragment thereof, comprises one or more of the amino acid sequences set forth in any one of Tables 1-11 (e.g., one or more of the sequences of DSL, DGL, or set forth in SEQ ID NOs: 12, 13, 14, 15, 16, 17, 18, 19, 20, 21, 22, 23, 24, 25, 26, 27, 28, 30, 31, 32, 33, 34, 35, 36, 37, 38, 39, 40, 41, 42, 43, 44, 45, 46, 47, 48, 49, 50, 51, 52, 53, 54, 55, 56, 57, 58, 61, 63, 65, 67, 69, 71, 75, 77, 79, 81, 83, 85, 87, 89, 91, 93, 95, 97, 99, and 101). In some embodiments, an antibody or antigen-binding fragment thereof, comprises a CDR set forth in any one of Tables 1-11 (e.g., any one of DSL, DGL, or SEQ ID NOs: 12, 13, 14, 15, 16, 17, 18, 19, 20, 21, 22, 23, 24, 25, 26, 27, 28, 30, 31, 32, 33, 34, 35, 36, 37, 38, 39, 40, 41, 42, 43, 44, 45, 46, 47, 48, 49, 50, 51, 52, 53, 54, 55, 56, 57, and 58). In some embodiments, a CDR (e.g., a heavy chain variable region (VH) CDR or a light chain variable region (VL) CDR comprises a CDR1 amino acid sequence set forth in any one of Tables 1-11 (see e.g., SEQ ID NOs: 12, 15, 18, 21, 24, 27, 30, 33, 36, 39, 42, 45, 48, 51, 54, and 57), a CDR2 amino acid sequence set forth in any one of Tables 1-11 (see e.g., SEQ ID NOs: 13, 16, 19, 22, 25, 28, 31, 34, 37, 40, 43, 46, 49, 52, 55, and 58), or a CDR3 amino acid sequence set forth in any one of Tables 1-11 (see e.g., DSL, DGL, or SEQ ID NOs: 14, 17, 20, 23, 26, 32, 35, 38, 41, 44, 47, 50, 53, and 56). In some embodiments, an antibody or antigen-binding fragment thereof, comprises a plurality of CDRs (e.g., at least one CDR1, at least one CDR2, and at least one CDR3), wherein one or more of the CDRs in the plurality comprises a CDR amino acid sequence set forth in any one of Tables 1-11. In some embodiments, the CDR1, CDR2, and/or CDR3 are selected from the same table (e.g., a V_{H}CDR1, V_{H} CDR2, and V_{H} CDR3 from the same table and/or a V_{L} CDR1, V_{L} CDR2, and V_{L} CDR3 from the same table) or a different table (e.g., a V_{H} CDR1, V_{H} CDR2, and V_{H} CDR3 which are selected from two or three different tables and/or a V_{L} CDR1, V_{L} CDR2, and V_{L} CDR3 which are selected from two or three different tables) selected from Tables 1-11. In some embodiments, an antibody or antigen-binding fragment thereof, comprises a VH. In some embodiments, the VH comprises a CDR1, a CDR2, and/or a CDR3 comprising a CDR amino acid sequence set forth in any one of Tables 1-11 (see, e.g., the V_{H} CDRs of DSL, DGL, or set forth in SEQ ID NOs: 15, 16, 21, 24, 27, 33, 36, 42, 45, 48, 54, 57, 22, 25, 28, 34, 37, 43, 46, 49, 55, 58, 17, 23, 26, 35, 38, 44, 47, 50, and 56, and the V_{L} CDRs set forth in SEQ ID NOs: 12, 13, 14, 18, 19, 20, 30, 31, 32, 39, 40, 41, 51, 52, and 53). In some embodiments, the CDR1, CDR2, and/or CDR3 of the VH are selected from the same table or two or more different tables selected from Tables 1-11. In some embodiments, the VH comprises a VH amino acid sequence set forth in any one of Tables 1-11 (see, e.g., SEQ ID NOs: 63, 69, 73, 75, 79, 83, 87, 91, 93, 97, and 101). In some embodiments, an antibody or antigen-binding fragment thereof, comprises a VL. In some embodiments, the VL comprises a CDR1, a CDR2, and/or a CDR3 set forth in any one of Tables 1-11 (see, e.g., the V_{H} CDRs of DSL, DGL, or set forth in SEQ ID NOs: 15, 16, 21, 24, 27, 33, 36, 42, 45, 48, 54, 57, 22, 25, 28, 34, 37, 43, 46, 49, 55, 58, 17, 23, 26, 35, 38, 44, 47, 50, and 56, and the V_{L} CDRs set forth in SEQ ID NOs: 12, 13, 14, 18, 19, 20, 30, 31, 32, 39, 40, 41, 51, 52, and 53). In some embodiments, the CDR1, CDR2, and/or CDR3 of the VL are selected from the same table (e.g., a V_{L} CDR1, V_{L} CDR2, and V_{L} CDR3 from the same table) or two or three different tables selected from Tables 1-11. In some embodiments, the VL comprises a VL amino acid sequence set forth in any one of Tables 1-11 (see, e.g., SEQ ID NOs: 61, 67, 77, 85, and 95). In some embodiments, an antibody or antigen-binding fragment thereof, comprises a VH and a VL set forth in any one of Tables 1-11, wherein the VH and VL are selected from the same table or a different table. In some embodiments, an antibody or antigen-binding fragment thereof, comprises a linker set forth in any one of Tables 1-11. In some embodiments, the linker connects a VH and a VL. In some embodiments, a chimeric antigen receptor described herein comprises an amino acid sequence set forth in any one of Tables 1-11. In some embodiments, a cell (e.g., a hematopoietic cell, such as a hematopoietic stem cell, a hematopoietic progenitor cell, or an immune cell, such as a T cell or a NK cell) comprises an antibody, or antigen binding fragment thereof, comprising an amino acid sequence set forth in any one of Tables 1-11. In some embodiments, a nucleic acid comprises a nucleotide sequence encoding an amino acid sequence set forth in any one of Tables 1-11. In some embodiments, a nucleic acid comprises a nucleotide sequence set forth in any one of Tables 1-11. In some embodiments, a cell (e.g., a hematopoietic cell, such as a hematopoietic stem cell, a hematopoietic progenitor cell, or an immune cell, such as a T cell or a NK cell) comprises nucleotide sequence set forth in any one Tables 1-11 (e.g., 60, 62, 64, 66, 68, 70, 72, 74, 76, 78, 80, 82, 84, 86, 88, 90, 92, 94, 96, 98, 100, and 102).

TABLE 1
Anti-EMR2 Clone EMR2-1-scFv

| Anti-EMR2 | |
|---|---|
| Clone EMR2-1- | |
| scFv | Sequence |
| V_{L} CDR1 | GGNNIGSKNVH (SEQ ID NO: 12) |
| V_{L} CDR2 | RDSNRPS (SEQ ID NO: 13) |
| V_{L} CDR3 | QVWDSSTVV (SEQ ID NO: 14) |
| V_{H} CDR1 | GYTFTSYGIS (SEQ ID NO: 15) |
| V_{H} CDR2 | WISAYNGNTNYAQKLQG (SEQ ID NO: 16) |
| V_{H} CDR3 | GYAFDI (SEQ ID NO: 17) |
| V_{L} Nucleotide | tcctatgagctgacacagccactctcagtgtcagtggccctgggacagacggccaggattacctgtgggggaaac |
| Sequence | aacattggaagtaaaaatgtgcactggtaccagcagaagccaggccaggcccctgtgctggtcatctatagggata |
| | gcaaccggccctctgggatccctgagcgattctctggctccaactcggggaacacggccaccctgaccatcaaca |
| | gagcccaagccggggatgaggctgactattactgtcaggtgtgggacagcagcactgtggtattcggcggaggg |
| | acggagctgaccgtccta (SEQ ID NO: 60) |
| V_{L} Amino Acid | SYELTQPLSVSVALGQTARITCGGNNIGSKNVHWYQQKPGQAPVLVIYRD |
| Sequence | SNRPSGIPERFSGSNSGNTATLTINRAQAGDEADYYCQVWDSSTVVFGGG |
| | TELTVL (SEQ ID NO: 61) |
| Linker Nucleotide | ggtggaggcggttccggaggcggtggcagcggcggtggeggttcg (SEQ ID NO: 102) |
| Sequence | |
| Linker Amino | GGGGSGGGGSGGGGS (SEQ ID NO: 3) |
| Acid Sequence | |
| V_{H} Nucleotide | caggttcagctggtgcagtctggagctgaggtgaagaagcctggggcctcagtgaaggtctcctgcaaggcttctg |
| Sequence | gttacacctttaccagctatggtatcagctgggtgcgacaggcccctggacaagggcttgagtggatgggatggat |
| | cagcgcttacaatggtaacacaaactatgcacagaagctccagggcagagtcaccatgaccacagacacatccac |
| | gagcacagcctacatggagctgaggagcctgagatctgacgacacggccgtgtattactgtgcgagagggtatgc |
| | ttttgatatctggggccaagggacaatggtcaccgtctcctct (SEQ ID NO: 62) |
| V_{H} Amino Acid | QVQLVQSGAEVKKPGASVKVSCKASGYTFTSYGISWVRQAPGQGLEWM |
| Sequence | GWISAYNGNTNYAQKLQGRVTMTTDTSTSTAYMELRSLRSDDTAVYYCA |
| | RGYAFDIWGQGTMVTVSS (SEQ ID NO: 63) |
| scFv Nucleotide | tcctatgagctgacacagccactctcagtgtcagtggccctgggacagacggccaggattacctgtgggggaaac |
| Sequence | aacattggaagtaaaaatgtgcactggtaccagcagaagccaggccaggcccctgtgctggtcatctatagggata |
| | gcaaccggccctctgggatccctgagcgattctctggctccaactcggggaacacggccaccctgaccatcaaca |
| | gagcccaagccggggatgaggctgactattactgtcaggtgtgggacagcagcactgtggtattcggcggaggg |
| | acggagctgaccgtcctaggtggaggcggttccggaggcggtggcagcggcggtggcggttcgcaggttcagc |
| | tggtgcagtctggagctgaggtgaagaagcctggggcctcagtgaaggtctcctgcaaggcttctggttacaccttt |
| | accagctatggtatcagctgggtgcgacaggcccctggacaagggcttgagtggatgggatggatcagcgcttac |
| | aatggtaacacaaactatgcacagaagctccagggcagagtcaccatgaccacagacacatccacgagcacagc |
| | ctacatggagctgaggagcctgagatctgacgacacggccgtgtattactgtgcgagagggtatgcttttgatatctg |
| | gggccaagggacaatggtcaccgtctcctca (SEQ ID NO: 64) |
| scFv Amino Acid | SYELTQPLSVSVALGQTARITCGGNNIGSKNVHWYQQKPGQAPVLVIYRD |
| Sequence | SNRPSGIPERFSGSNSGNTATLTINRAQAGDEADYYCQVWDSSTVVFGGG |
| | TELTVLGGGGSGGGGSGGGGSQVQLVQSGAEVKKPGASVKVSCKASGY |
| | TFTSYGISWVRQAPGQGLEWMGWISAYNGNTNYAQKLQGRVTMTTDTS |
| | TSTAYMELRSLRSDDTAVYYCARGYAFDIWGQGTMVTVSS (SEQ ID NO: |
| | 65) |

TABLE 2
Anti-EMR2 Clone EMR2-2-scFv

| Anti-EMR2 Clone | |
|---|---|
| EMR2-2-scFv | Sequence |
| V_{L} CDR1 | RASQSISSYLN (SEQ ID NO: 18) |
| V_{L} CDR2 | AASSLQS (SEQ ID NO: 19) |
| V_{L} CDR3 | QQSYSTPPWT (SEQ ID NO: 20) |
| V_{H} CDR1 | GFTFSSYEMN (SEQ ID NO: 21) |
| V_{H} CDR2 | AISGGGGSTYYADSVKG (SEQ ID NO: 22) |
| V_{H} CDR3 | SGRGWAAAGR (SEQ ID NO: 23) |
| V_{L} Nucleotide | gccatccagatgacccagtctccatcctccctgtctgcatctgtaggagacagagtcaccatcacttgccgggcaag |
| Sequence | tcagagcattagcagctatttaaattggtatcagcagaaaccagggaaagcccctaagctcctgatctatgctgcatc |
| | cagtttgcaaagtggggtcccatcaaggttcagtggcagtggatctgggacagatttcactctcaccatcagcagtct |
| | gcaacctgaagattttgcaacttactactgtcaacagagttacagtacccctccgtggacgttcggccaagggacca |
| | aggtggagatcaaa (SEQ ID NO: 66) |
| V_{L} Amino Acid | AIQMTQSPSSLSASVGDRVTITCRASQSISSYLNWYQQKPGKAPKLLIYAA |
| Sequence | SSLQSGVPSRFSGSGSGTDFTLTISSLQPEDFATYYCQQSYSTPPWTFGQG |
| | TKVEIK (SEQ ID NO: 67) |
| Linker Nucleotide | ggtggaggcggttccggaggcggtggcagcggcggtggcggttcg (SEQ ID NO: 102) |
| Sequence | |
| Linker Amino | GGGGSGGGGSGGGGS (SEQ ID NO: 3) |
| Acid Sequence | |
| V_{H} Nucleotide | CAGGTGCAGCCGGTGGAGTCTGGGGGAGGCTTGGTACAGCCTGGAGG |
| Sequence | GTCCCTGAGACTCTCCTGTGCAGCCTCTGGATTCACCTTCAGTAGTTAT |
| | GAAATGAACTGGGTCCGCCAGGCTCCAGGGAAGGGGCTGGAGTGGGT |
| | CTCAGCTATTAGTGGTGGTGGTGGTAGCACATACTACGCAGACTCCGT |
| | GAAGGGCCGGTTCACCATCTCCAGAGACAATTCCAAGAACACGCTGT |
| | ATCTGCAGATGAACAGCCTGAGAGCCGAGGACACGGCCGTATATTACT |
| | GTGCGAAATCCGGGCGCGGGTGGGCAGCAGCTGGTAGATGGGGCCAG |
| | GGCACCCTGGTCACCGTCTCCTCA (SEQ ID NO: 68) |
| V_{H} Amino Acid | QVQPVESGGGLVQPGGSLRLSCAASGFTFSSYEMNWVRQAPGKGLEWV |
| Sequence | SAISGGGGSTYYADSVKGRFTISRDNSKNTLYLQMNSLRAEDTAVYYCA |
| | KSGRGWAAAGRWGQGTLVTVSS (SEQ ID NO: 69) |
| scFv Nucleotide | GCCATCCAGATGACCCAGTCTCCATCCTCCCTGTCTGCATCTGTAGGAG |
| Sequence | ACAGAGTCACCATCACTTGCCGGGCAAGTCAGAGCATTAGCAGCTATT |
| | TAAATTGGTATCAGCAGAAACCAGGGAAAGCCCCTAAGCTCCTGATCT |
| | ATGCTGCATCCAGTTTGCAAAGTGGGGTCCCATCAAGGTTCAGTGGCA |
| | GTGGATCTGGGACAGATTTCACTCTCACCATCAGCAGTCTGCAACCTG |
| | AAGATTTTGCAACTTACTACTGTCAACAGAGTTACAGTACCCCTCCGT |
| | GGACGTTCGGCCAAGGGACCAAGGTGGAGATCAAAGGTGGAGGCGG |
| | TTCCGGAGGCGGTGGCAGCGGCGGTGGCGGTTCGCAGGTGCAGCCGG |
| | TGGAGTCTGGGGGAGGCTTGGTACAGCCTGGAGGGTCCCTGAGACTC |
| | TCCTGTGCAGCCTCTGGATTCACCTTCAGTAGTTATGAAATGAACTGG |
| | GTCCGCCAGGCTCCAGGGAAGGGGCTGGAGTGGGTCTCAGCTATTAG |
| | TGGTGGTGGTGGTAGCACATACTACGCAGACTCCGTGAAGGGCCGGTT |
| | CACCATCTCCAGAGACAATTCCAAGAACACGCTGTATCTGCAGATGAA |
| | CAGCCTGAGAGCCGAGGACACGGCCGTATATTACTGTGCGAAATCCGG |
| | GCGCGGGTGGGCAGCAGCTGGTAGATGGGGCCAGGGCACCCTGGTCA |
| | CCGTCTCCTCA (SEQ ID NO: 70) |
| scFv Amino Acid | AIQMTQSPSSLSASVGDRVTITCRASQSISSYLNWYQQKPGKAPKLLIYAA |
| Sequence | SSLQSGVPSRFSGSGSGTDFTLTISSLQPEDFATYYCQQSYSTPPWTFGQG |
| | TKVEIKGGGGSGGGGSGGGGSQVQPVESGGGLVQPGGSLRLSCAASGFT |
| | FSSYEMNWVRQAPGKGLEWVSAISGGGGSTYYADSVKGRFTISRDNSKN |
| | TLYLQMNSLRAEDTAVYYCAKSGRGWAAAGRWGQGTLVTVSS |
| | (SEQ ID NO: 71) |

TABLE 3
Anti-EMR2 Clone EMR2-3-VH

| Anti-EMR2 Clone | |
|---|---|
| EMR2-3-VH | Sequence |
| V_{H} CDR1 | DFSFDSYEMS (SEQ ID NO: 24) |
| V_{H} CDR2 | SISASGGRTFYADSVKG (SEQ ID NO: 25) |
| V_{H} CDR3 | HPGI (SEQ ID NO: 26) |
| V_{H} Nucleotide | caggtgcagctggtgcagtctgggggaggcttggtacagcctggagggtccctgagactctcctgtgcagcctctg |
| Sequence | atttctctttcgattcttatgaaatgagctgggtccgccaggctccagggaaggggctagagtgggtctcaagtatta |
| | gtgctagtggtggtaggacattctacgcagactctgtgaagggccgattcaccatctccagagacaattccaagaac |
| | acgctgtatctgcaaatgaacaccctgagagccgaggacacagccttgtattactgtgcgagacatccggggattat |
| | gggccagggaaccctggtcaccgtctcctca (SEQ ID NO: 72) |
| V_{H} Amino Acid | QVQLVQSGGGLVQPGGSLRLSCAASDFSFDSYEMSWVRQAPGKGLEWV |
| Sequence | SSISASGGRTFYADSVKGRFTISRDNSKNTLYLQMNTLRAEDTALYYCAR |
| | HPGIMGQGTLVTVSS (SEQ ID NO: 73) |

TABLE 4
Anti-EMR2 Clone EMR2-4-VH

| Anti-EMR2 Clone | |
|---|---|
| EMR2-4-VH | Sequence |
| V_{H} CDR1 | SFDFASYEMS (SEQ ID NO: 27) |
| V_{H} CDR2 | SISGSGGHIFYADSVKG (SEQ ID NO: 28) |
| V_{H} CDR3 | DGL |
| V_{H} Nucleotide | caggtgcagctggtgcagtctgggggaggcttggtacagcctggagggtccctgagactctcctgtgcagcctctt |
| Sequence | ctttcgatttcgcttcttatgaaatgagctgggtccgccaggctccagggaagggactagagtgggtctcatcgatta |
| | gtggtagtggtggtcacatattctacgcagactccgtgaagggccgattcaccatctccagagacaattccaagaac |
| | acgctgtatctgcaaatgaacaccctgagagccgaggacacggccgtgtattactgtgcgagagatggattgatgg |
| | gccagggaaccctggtcaccgtctcctca (SEQ ID NO: 74) |
| V_{H} Amino Acid | QVQLVQSGGGLVQPGGSLRLSCAASSFDFASYEMSWVRQAPGKGLEWV |
| Sequence | SSISGSGGHIFYADSVKGRFTISRDNSKNTLYLQMNTLRAEDTAVYYCAR |
| | DGLMGQGTLVTVSS (SEQ ID NO: 75) |

TABLE 5
Anti-EMR2 Clone EMR2-5-scFv

| Anti-EMR2 Clone | |
|---|---|
| EMR2-5-scFv | Sequence |
| V_{L} CDR1 | GGNNIGSKNVH (SEQ ID NO: 30) |
| V_{L} CDR2 | RDSNRPS (SEQ ID NO: 31) |
| V_{L} CDR3 | QVWDSSTGV (SEQ ID NO: 32) |
| V_{H} CDR1 | GYTFTSYGIS (SEQ ID NO: 33) |
| V_{H} CDR2 | WISAYNGNTNYAQKLQG (SEQ ID NO: 34) |
| V_{H} CDR3 | GYSYGL (SEQ ID NO: 35) |
| V_{L} Nucleotide | tcctatgtgctgactcagccactctcagtgtcagtggccctgggacagacggccaggattacctgtgggggaaaca |
| Sequence | acattggaagtaaaaatgtgcactggtaccagcagaagccaggccaggcccctgtgctggtcatctatagggatag |
| | caaccggccctctgggatccctgagcgattctctggctccaactcggggaacacggccatcctgaccatcagcag |
| | agcccaagccggggatgaggctgactattactgtcaggtgtgggacagcagcactggggtgttcggcggaggaa |
| | cccagctgatcatttta (SEQ ID NO: 76) |
| V_{L} Amino Acid | SYVLTQPLSVSVALGQTARITCGGNNIGSKNVHWYQQKPGQAPVLVIYR |
| Sequence | DSNRPSGIPERFSGSNSGNTAILTISRAQAGDEADYYCQVWDSSTGVFGG |
| | GTQLIIL (SEQ ID NO: 77) |
| Linker Nucleotide | ggaggaggcggttccggaggcggtggcagcggcggtggcggttcg (SEQ ID NO: 103) |
| Sequence | |
| Linker Amino | GGGGSGGGGSGGGGS (SEQ ID NO: 3) |
| Acid Sequence | |
| V_{H} Nucleotide | Caggtgcagctggtgcagtctggagctgaggtgaagaagcctggggcctcagtgaaggtctcctgcaaggcttct |
| Sequence | ggttacacctttaccagctatggtatcagctgggtgcgacaggcccctggacaagggcttgagtggatgggatgga |
| | tcagcgcttacaatggtaacacaaactatgcacagaagctccagggcagggtcaccatgaccacagacacatcca |
| | cgagcacagcctacatggagctgaggagcctgagatctgacgacacggccgtgtattactgtgcgagagggtaca |
| | gctatggtctctggggccagggaaccacggtcaccgtctcctca (SEQ ID NO: 78) |
| V_{H} Amino Acid | QVQLVQSGAEVKKPGASVKVSCKASGYTFTSYGISWVRQAPGQGLEWM |
| Sequence | GWISAYNGNTNYAQKLQGRVTMTTDTSTSTAYMELRSLRSDDTAVYYCA |
| | RGYSYGLWGQGTTVTVSS (SEQ ID NO: 79) |
| scFv Nucleotide | Tcctatgtgctgactcagccactctcagtgtcagtggccctgggacagacggccaggattacctgtgggggaaac |
| Sequence | aacattggaagtaaaaatgtgcactggtaccagcagaagccaggccaggcccctgtgctggtcatctatagggata |
| | gcaaccggccctctgggatccctgagcgattctctggctccaactcggggaacacggccatcctgaccatcagca |
| | gagcccaagccggggatgaggctgactattactgtcaggtgtgggacagcagcactggggtgttcggcggagga |
| | acccagctgatcattttaggaggaggcggttccggaggcggtggcagcggcggtggcggttcgcaggtgcagct |
| | ggtgcagtctggagctgaggtgaagaagcctggggcctcagtgaaggtctcctgcaaggcttctggttacaccttta |
| | ccagctatggtatcagctgggtgcgacaggcccctggacaagggcttgagtggatgggatggatcagcgcttaca |
| | atggtaacacaaactatgcacagaagctccagggcagggtcaccatgaccacagacacatccacgagcacagcc |
| | tacatggagctgaggagcctgagatctgacgacacggccgtgtattactgtgcgagagggtacagctatggtctct |
| | ggggccagggaaccacggtcaccgtctcctca (SEQ ID NO: 80) |
| scFv Amino Acid | SYVLTQPLSVSVALGQTARITCGGNNIGSKNVHWYQQKPGQAPVLVIYR |
| Sequence | DSNRPSGIPERFSGSNSGNTAILTISRAQAGDEADYYCQVWDSSTGVFGG |
| | GTQLIILGGGGSGGGGSGGGGSQVQLVQSGAEVKKPGASVKVSCKASG |
| | YTFTSYGISWVRQAPGQGLEWMGWISAYNGNTNYAQKLQGRVTMTTDT |
| | STSTAYMELRSLRSDDTAVYYCARGYSYGLWGQGTTVTVSS (SEQ ID NO: 81) |

TABLE 6
Anti-EMR2 Clone EMR2-6-VH

| Anti-EMR2 Clone | |
|---|---|
| EMR2-6-VH | Sequence |
| V_{H} CDR1 | DFSFSAYEMS (SEQ ID NO: 36) |
| V_{H} CDR2 | SISGSGRSTYYADSVKG (SEQ ID NO: 37) |
| V_{H} CDR3 | GSGYYDSSGYL (SEQ ID NO: 38) |
| V_{H} Nucleotide | caggtgcagctggtgcagtctgggggaggcctggtacagcctggagggtccctgagactctcctgtgcagcctct |
| Sequence | gatttctctttctctgcttatgaaatgagctgggtccgccaggctccagggaagggcctagagtgggtctcaagtatta |
| | gtggtagtggtcgtagcacatactacgcagactccgtgaagggccgattcaccatctccagagacaattccaagaa |
| | cacgctgtatctgcaaatgaacaccctgagagccgaggacacagccatgtattactgtgcgaaaggtagcggttac |
| | tatgatagtagtggttacctctggggccagggaaccctggtcaccgtctcctca (SEQ ID NO: 82) |
| V_{H} Amino Acid | QVQLVQSGGGLVQPGGSLRLSCAASDFSFSAYEMSWVRQAPGKGLEWV |
| Sequence | SSISGSGRSTYYADSVKGRFTISRDNSKNTLYLQMNTLRAEDTAMYYCA |
| | KGSGYYDSSGYLWGQGTLVTVSS (SEQ ID NO: 83) |

TABLE 7
Anti-EMR2 Clone EMR2-7-scFv

| Anti-EMR2 Clone | |
|---|---|
| EMR2-7-scFv | Sequence |
| V_{L} CDR1 | QGDSLRTYYAS (SEQ ID NO: 39) |
| V_{L} CDR2 | GKNNRPS (SEQ ID NO: 40) |
| V_{L} CDR3 | QTWDSTTGV (SEQ ID NO: 41) |
| V_{H} CDR1 | GFTFSNYEMN (SEQ ID NO: 42) |
| V_{H} CDR2 | AISGSGGSTYYADSVKG (SEQ ID NO: 43) |
| V_{H} CDR3 | DGGGWFGEEY (SEQ ID NO: 44) |
| V_{L} Nucleotide | tcttctgagctgactcaggaccctgctgtgtctgtggccttgggacagacagtcaggatcacatgccaaggagaca |
| Sequence | gcctcagaacctattatgcaagctggtaccagcagaagccaggacaggcccctgtacttgtcatctatggtaaaaac |
| | aaccggccctcagggatccctgagcgattctctggctccaactctgggaacatagccactctgaccatcagcggga |
| | cccagcctatggatgaggctgactattactgtcagacgtgggacagtaccactggcgtcttcggcggagggaccg |
| | agctgaccgtccta (SEQ ID NO: 84) |
| V_{L} Amino Acid | SSELTQDPAVSVALGQTVRITCQGDSLRTYYASWYQQKPGQAPVLVIYGK |
| Sequence | NNRPSGIPERFSGSNSGNIATLTISGTQPMDEADYYCQTWDSTTGVFGGG |
| | TELTVL (SEQ ID NO: 85) |
| Linker Nucleotide | ggtggaggcggttccggaggcggtggcagcggcggtggcggttcg (SEQ ID NO: 102) |
| Sequence | |
| Linker Amino | GGGGSGGGGSGGGGS (SEQ ID NO: 3) |
| Acid Sequence | |
| V_{H} Nucleotide | caggtgcagctggtggagtctgggggaggcttggtacagcctggagggtccctgagactctcctgtgcaacctctg |
| Sequence | gattcaccttcagtaattatgaaatgaactgggtccgccaggctccagggaaggggctggagtgggtctcagctatt |
| | agtggtagtggtggtagcacatactacgcagactccgtgaagggccggttcaccatctccagagacaattccaaga |
| | acacgctgtatctgcaaatgaacagcctgagagccgaggacacggccgtatattactgtgcgaaagatgggggcg |
| | gttggttcggggaggagtactggggccagggagccacggtcaccgtctcctca (SEQ ID NO: 86) |
| V_{H} Amino Acid | QVQLVESGGGLVQPGGSLRLSCATSGFTFSNYEMNWVRQAPGKGLEWV |
| Sequence | SAISGSGGSTYYADSVKGRFTISRDNSKNTLYLQMNSLRAEDTAVYYCAK |
| | DGGGWFGEEYWGQGATVTVSS (SEQ ID NO: 87) |
| scFv Nucleotide | tcttctgagctgactcaggaccctgctgtgtctgtggccttgggacagacagtcaggatcacatgccaaggagaca |
| Sequence | gcctcagaacctattatgcaagctggtaccagcagaagccaggacaggcccctgtacttgtcatctatggtaaaaac |
| | aaccggccctcagggatccctgagcgattctctggctccaactctgggaacatagccactctgaccatcagcggga |
| | cccagcctatggatgaggctgactattactgtcagacgtgggacagtaccactggcgtcttcggcggagggaccg |
| | agctgaccgtcctaggtggaggcggttccggaggcggtggcagcggcggtggcggttcgcaggtgcagctggt |
| | ggagtctgggggaggcttggtacagcctggagggtccctgagactctcctgtgcaacctctggattcaccttcagta |
| | attatgaaatgaactgggtccgccaggctccagggaaggggctggagtgggtctcagctattagtggtagtggtgg |
| | tagcacatactacgcagactccgtgaagggccggttcaccatctccagagacaattccaagaacacgctgtatctg |
| | caaatgaacagcctgagagccgaggacacggccgtatattactgtgcgaaagatgggggcggttggttcgggga |
| | ggagtactggggccagggagccacggtcaccgtctcctca (SEQ ID NO: 88) |
| scFv Amino Acid | SSELTQDPAVSVALGQTVRITCQGDSLRTYYASWYQQKPGQAPVLVIYGK |
| Sequence | NNRPSGIPERFSGSNSGNIATLTISGTQPMDEADYYCQTWDSTTGVFGGG |
| | TELTVLGGGGSGGGGSGGGGSQVQLVESGGGLVQPGGSLRLSCATSGFT |
| | FSNYEMNWVRQAPGKGLEWVSAISGSGGSTYYADSVKGRFTISRDNSKN |
| | TLYLQMNSLRAEDTAVYYCAKDGGGWFGEEYWGQGATVTVSS (SEQ ID NO: 89) |

TABLE 8
Anti-EMR2 Clone EMR2-8-VH

| Anti-EMR2 Clone | |
|---|---|
| EMR2-8-VH | Sequence |
| V_{H} CDR1 | AFAFADYEMS (SEQ ID NO: 45) |
| V_{H} CDR2 | AISGSGGSTYYADSVKG (SEQ ID NO: 46) |
| V_{H} CDR3 | GTAYGDYENY (SEQ ID NO: 47) |
| V_{H} Nucleotide | caggtgcagctggtgcagtctgggggaggcttggtacagcctggagggtccctgagactctcctgtgcagcctctg |
| Sequence | ctttcgctttcgctgattatgaaatgagctgggtccgccaggctccaggacaaggccttgagtgggtctcagctatta |
| | gtggtagtggtggtagcacatactacgcagactccgtgaagggccgatttaccatctccagagacaattccaagaa |
| | cacgctgtatctgcaaatgaacaccctgagagccgaggacacggccgtgtattactgtgcgagaggcacagccta |
| | cggtgactacgaaaactactggggccagggaaccctggtcaccgtctcctca (SEQ ID NO: 90) |
| V_{H} Amino Acid | QVQLVQSGGGLVQPGGSLRLSCAASAFAFADYEMSWVRQAPGQGLEWV |
| Sequence | SAISGSGGSTYYADSVKGRFTISRDNSKNTLYLQMNTLRAEDTAVYYCAR |
| | GTAYGDYENYWGQGTLVTVSS (SEQ ID NO: 91) |

TABLE 9
Anti-EMR2 Clone EMR2-9-VH

| Anti-EMR2 Clone | |
|---|---|
| EMR2-9-VH | Sequence |
| V_{H} CDR1 | AFDFDDAYMD (SEQ ID NO: 48) |
| V_{H} CDR2 | GINWNSGWVGYADSVKG (SEQ ID NO: 49) |
| V_{H} CDR3 | TGIAAAGTNFDY (SEQ ID NO: 50) |
| V_{H} Nucleotide | caggtgcagctggtgcagtctgggggaggcttggtacagcctggagggtccctgagactctcctgtgcagcctctg |
| Sequence | ctttcgatttcgatgatgcttatatggactgggtccgccaggctccagggaaggggctagagtgggtcgcaggtatt |
| | aattggaatagtggttgggtaggctatgcggactcagtgaagggccgattcaccatctccagagacaattccaaga |
| | acacgctgtatctgcaaatgaacaccctgagagccgaggacacggccgtatattactgtgcgagaacgggtatag |
| | cagcagctggtaccaactttgactactggggccagggaaccctggtcactgtctcctca (SEQ ID NO: 92) |
| V_{H} Amino Acid | QVQLVQSGGGLVQPGGSLRLSCAASAFDFDDAYMDWVRQAPGKGLEW |
| Sequence | VAGINWNSGWVGYADSVKGRFTISRDNSKNTLYLQMNTLRAEDTAVYY |
| | CARTGIAAAGTNFDYWGQGTLVTVSS (SEQ ID NO: 93) |

TABLE 10
Anti-EMR2 Clone EMR2-10-scFv

| Anti-EMR2 Clone | |
|---|---|
| EMR2-10-scFv | Sequence |
| V_{L} CDR1 | SGSSSNIGNNYVS (SEQ ID NO: 51) |
| V_{L} CDR2 | DNNKRPS (SEQ ID NO: 52) |
| V_{L} CDR3 | GTWDSSLSAWV (SEQ ID NO: 53) |
| V_{H} CDR1 | GYTFTGYYMH (SEQ ID NO: 54) |
| V_{H} CDR2 | IINPSGGSTIYAQKFQG (SEQ ID NO: 55) |
| V_{H} CDR3 | VKYSKLNDAFDI (SEQ ID NO: 56) |
| V_{L} Nucleotide | cagtctgtgttgactcagccgccctcagtgtctgcggccccaggacagaaggtcaccatctcctgctctggaagca |
| Sequence | gctccaacattgggaataattatgtttcctggtaccagcacctcccaggaacagcccccaaactcctcatttatgaca |
| | ataataagcgaccctcagggattcctgaccgattctctggctccaagtctgccacgtcagccaccctgggcatcacc |
| | ggactccagactggggacgaggccgattattactgcggaacatgggatagcagcctgagtgcttgggtgttcggc |
| | ggagggaccaagctgaccgtccta (SEQ ID NO: 94) |
| V_{L} Amino Acid | QSVLTQPPSVSAAPGQKVTISCSGSSSNIGNNYVSWYQHLPGTAPKLLIYD |
| Sequence | NNKRPSGIPDRFSGSKSATSATLGITGLQTGDEADYYCGTWDSSLSAWVF |
| | GGGTKLTVL (SEQ ID NO: 95) |
| Linker Nucleotide | ggtggaggcggttccggaggcggtggcagcggcggtggcggttcg (SEQ ID NO: 102) |
| Sequence | |
| Linker Amino | GGGGSGGGGSGGGGS (SEQ ID NO: 3) |
| Acid Sequence | |
| V_{H} Nucleotide | caggtgcagctggtgcagtctggggctgaggtgaagaagcctggggcctcagtgaaggtctcctgcaaggcttct |
| Sequence | ggatacaccttcaccggctactatatgcactgggtgcgacaggcccctggacaagggcttgagtggatgggaata |
| | atcaaccctagtggtggtagcacaatctacgcacagaagttccagggcagagtcaccatgaccagggacacgtcc |
| | acgagcacagtctacatggagctgagcagcctgggatctgaggacacggccgtgtattactgtgcgagagtaaaa |
| | tacagcaagcttaatgatgcttttgatatctggggccaagggacaatggtcaccgtctcttca (SEQ ID NO: 96) |
| V_{H} Amino Acid | QVQLVQSGAEVKKPGASVKVSCKASGYTFTGYYMHWVRQAPGQGLEW |
| Sequence | MGIINPSGGSTIYAQKFQGRVTMTRDTSTSTVYMELSSLGSEDTAVYYCA |
| | RVKYSKLNDAFDIWGQGTMVTVSS (SEQ ID NO: 97) |
| scFv Nucleotide | cagtctgtgttgactcagccgccctcagtgtctgcggccccaggacagaaggtcaccatctcctgctctggaagca |
| Sequence | gctccaacattgggaataattatgtttcctggtaccagcacctcccaggaacagcccccaaactcctcatttatgaca |
| | ataataagcgaccctcagggattcctgaccgattctctggctccaagtctgccacgtcagccaccctgggcatcacc |
| | ggactccagactggggacgaggccgattattactgcggaacatgggatagcagcctgagtgcttgggtgttcggc |
| | ggagggaccaagctgaccgtcctaggtggaggcggttccggaggcggtggcagcggcggtggcggttcgcag |
| | gtgcagctggtgcagtctggggctgaggtgaagaagcctggggcctcagtgaaggtctcctgcaaggcttctgga |
| | tacaccttcaccggctactatatgcactgggtgcgacaggcccctggacaagggcttgagtggatgggaataatca |
| | accctagtggtggtagcacaatctacgcacagaagttccagggcagagtcaccatgaccagggacacgtccacg |
| | agcacagtctacatggagctgagcagcctgggatctgaggacacggccgtgtattactgtgcgagagtaaaataca |
| | gcaagcttaatgatgcttttgatatctggggccaagggacaatggtcaccgtctcttca (SEQ ID NO: 98) |
| scFv Amino Acid | QSVLTQPPSVSAAPGQKVTISCSGSSSNIGNNYVSWYQHLPGTAPKLLIYD |
| Sequence | NNKRPSGIPDRFSGSKSATSATLGITGLQTGDEADYYCGTWDSSLSAWVF |
| | GGGTKLTVLGGGGSGGGGSGGGGSQVQLVQSGAEVKKPGASVKVSCK |
| | ASGYTFTGYYMHWVRQAPGQGLEWMGIINPSGGSTIYAQKFQGRVTMT |
| | RDTSTSTVYMELSSLGSEDTAVYYCARVKYSKLNDAFDIWGQGTMVTVS |
| | S (SEQ ID NO: 99) |

TABLE 11
Anti-EMR2 Clone EMR2-11-VH

| Anti-EMR2 Clone | |
|---|---|
| EMR2-11-VH | Sequence |
| V_{H} CDR1 | SFAFDSYEMS (SEQ ID NO: 57) |
| V_{H} CDR2 | SISGSGGSTYYADSVKG (SEQ ID NO: 58) |
| V_{H} CDR3 | DSL |
| V_{H} Nucleotide | caggtgcagctggtgcagtctgggggaggcttggtacagcctggagggtccctgagactctcctgtgcagcctctt |
| Sequence | ctttcgctttcgattcttatgaaatgagctgggtccgccaggctccaggacaaggccttgagtgggtctcatctattagt |
| | ggtagtggtggtagcacatactacgcagactccgtgaagggccgattcaccatctccagagacaattccaagaaca |
| | cgctgtatctgcaaatgaacaccctgagagccgaggacacggccgtatattactgtgcgagagattcccttatgggc |
| | cagggaaccctggtcaccgtctcctca (SEQ ID NO: 100) |
| V_{H} Amino Acid | QVQLVQSGGGLVQPGGSLRLSCAASSFAFDSYEMSWVRQAPGQGLEWV |
| Sequence | SSISGSGGSTYYADSVKGRFTISRDNSKNTLYLQMNTLRAEDTAVYYCAR |
| | DSLMGQGTLVTVSS (SEQ ID NO: 101) |

### Fusion Proteins and Conjugates

In some embodiments, the disclosure provides fusion proteins comprising (i) one or more single domain antibodies, or antigen-binding fragments thereof, described herein (e.g., comprising one or more CDRs described herein), and (ii) one or more additional polypeptides. In some embodiments, the disclosure provides fusion proteins comprising (i) one or more single domain antibodies, or antigen-binding fragments thereof, described herein (e.g., comprising one or more CDRs described herein), and (ii) one or more additional domains. For example, a fusion protein can include one or more single domain antibodies described herein and one or more (e.g., 1, 2, 3, 4 or more) constant regions or an Fc region. In some embodiments, one or more single domain antibodies, or antigen-binding fragments thereof, described herein (e.g., one or more CDRs described herein) can be conjugated non-covalently or covalently, e.g., fused, to an antigen (e.g., an antigen target for a cellular therapeutic, e.g., a CAR-T cell or an antibody drug conjugate) as described in, e.g., PCT Publication Nos. WO2017/075537, WO2017/075533, WO2018156802, and WO2018156791.

In some embodiments, the disclosure provides a fusion protein comprising one or more VHH as described herein and one or more additional polypeptides or polypeptide domains. In some embodiments, an additional polypeptide comprises an additional antibody or fragment thereof. Additional antibodies include, e.g., intact IgG, IgE and IgM, bi- or multi-specific antibodies (e.g., Zybodies®, etc.), single chain Fvs, polypeptide-Fc fusions, Fabs, cameloid antibodies, masked antibodies (e.g., Probodies®), Small Modular ImmunoPharmaceuticals (“SMIPs™”), single chain or Tandem diabodies (TandAb®), VHHs (including but not limited to those described in the present disclosure), Anticalins®, Nanobodies®, minibodies, BiTE®s, ankyrin repeat proteins or DARPINs®, Avimers®, a DART, a TCR-like antibody, Adnectins®, Affilins®, Trans-bodies®, Affibodies®, a TrimerX®, MicroProteins, Fynomers®, and Centyrins®.

In some embodiments, the one or more additional polypeptides or polypeptide domains comprises a second antigen-binding domain, such as a second antigen-binding domain that binds to the same target antigen (i.e., EMR2), for example any of the anti-EMR2 antibody, or antigen-binding fragments thereof, described herein. In some embodiments, the one or more additional polypeptides or polypeptide domains comprises a second antigen-binding domain, such as a second antigen-binding domain that binds to a different target antigen (e.g., not an epitope of EMR2).

In some embodiments, an antibody of the disclosure can be covalently attached by a linker (e.g., by a disulfide or non-cleavable thioether linker) to a drug (e.g., a cytotoxic agent, such as a toxin) as an antibody drug conjugate (ADC). The drug to which the antibody is covalently attached may have cytotoxic or cytostatic effect when it is not conjugated to the antibody. The ADC can be used to selectively deliver an effective dose of a cytotoxic agent to cells (e.g., to tumor tissue). The ADC may improve the bioavailability of the drug and/or the antibody compared to when the drug and/or antibody is administered in its unconjugated form.

A variety of linker types and strategies are known in the art and any or all of these are contemplated for use with the antibodies or ADCs of the disclosure. In some embodiments, the linker is biodegradable, e.g., cleavable by an endogenous protease (e.g., present in the target tissue and/or cells). In some embodiments, the linker comprises a protease cleavable site. In some embodiments, the linker comprises a pH sensitive site, e.g., a site sensitive to acidic pH, e.g., that hydrolyzes under acidic conditions. In some embodiments, the linker is stable under physiological conditions, e.g., sufficiently stable so that the antibody targets the drug to the target tissue prior to release of the drug. In some embodiments, the linker comprises a disulfide bond, e.g., a glutathione-sensitive disulfide bond. In some embodiments, the drug conjugated to the antibody is active only after cleavage of the linker. In some embodiments, the drug conjugated to the antibody is active only after proteolytic digestion of the antibody (e.g., in the lysosome of a target cell). In some embodiments, the linker is a non-cleavable heterobifunctional thioether linker, e.g., a maleimide linker, e.g., comprising N-hydroxysuccinimide ester (succinimidyl-4-(N-maleimidomethyl) cyclohexane-1-carboxylate or SMCC.

A variety of drugs compatible with ADCs of the disclosure are known in the art and any or all of these are contemplated for use with the antibodies of the disclosure.

Also within the scope of the present disclosure are chimeric antigen receptors (CARs) comprising any of the anti-EMR2 antibodies, or antigen-binding fragments thereof, described herein. A CAR is an artificially constructed hybrid protein or polypeptide containing the antigen-binding domain of one or more antibodies (e.g., single chain variable fragment (scFv)) linked to T-cell signaling domains. Characteristics of CARs include their ability to redirect T-cell specificity and reactivity toward a selected target in a non-MHC-restricted manner, exploiting the antigen-binding properties of monoclonal antibodies. The non-MHC-restricted antigen recognition gives T cells expressing CARs the ability to recognize antigen independent of antigen processing, thus bypassing a major mechanism of tumor escape. Moreover, when expressed in T-cells, CARs advantageously do not dimerize with endogenous T cell receptor (TCR) alpha and beta chains. The phrases “antigen(ic) specificity” and “elicit antigen-specific response,” as used herein, means that the CAR can specifically bind to and immunologically recognize an antigen, such that binding of the CAR to the antigen elicits an immune response.

Of the conventional CARs containing an antigen-binding domain of an antibody, there are three generations of CARs. “First generation” CARs are typically composed of an extracellular antigen-binding domain (e.g., a scFv), which is fused to a transmembrane domain, which is fused to cytoplasmic/intracellular signaling domain. First generation CARs can provide de novo antigen recognition and cause activation of both CD4+ and CD8+ T cells through their CD3ξ chain signaling domain in a single fusion molecule, independent of HLA-mediated antigen presentation. “Second generation” CARs add an intracellular signaling domain from various co-stimulatory signaling molecules (e.g., CD28, 4-1BB, ICOS, OX40, CD27, CD40/My88, and NKGD2) to the cytoplasmic tail of the CAR to provide additional signals to the T cell. Second generation CARs comprise those that provide both co-stimulation (e.g., CD28 or 4-1BB) and activation (CD3ξ). “Third generation” CARs comprise those that provide multiple co-stimulatory domains (e.g., CD28 and 4-1BB) and a signaling domain providing activation (e.g., CD3ξ).

The CARs described herein comprise an extracellular portion of the CAR containing an anti-EMR2 binding fragment, a transmembrane domain, and a signaling domain. In some embodiments, the CAR further comprises one or more of a linker region, hinge region, and co-stimulatory signaling domains. In some embodiments, the CAR further comprises a signal peptide/signal sequence.

CARs of the present disclosure (including functional portions and functional variants) can be of any length, i.e., can comprise any number of amino acids, provided that the CARs (or functional portions or functional variants thereof) retain their biological activity, e.g., the ability to specifically bind to the target antigen (e.g., EMR2), detect diseased cells in a mammal, or treat or prevent disease in a mammal. For example, the CAR can be about 50 to about 5000 amino acids long, such as 50, 70, 75, 100, 125, 150, 175, 200, 300, 400, 500, 600, 700, 800, 900, 1000 or more amino acids in length.

In some embodiments, CAR constructs (including functional portions and functional variants of the invention) can comprise synthetic amino acids in place of one or more naturally-occurring amino acids. Such synthetic amino acids are known in the art, and include, for example, aminocyclohexane carboxylic acid, norleucine, a-amino n-decanoic acid, homoserine, S-acetylaninomrethyl-cysteine, trans-3- and trans-4-hydroxyproline, 4-arninophenylalanine, 4-nitrophenylalanine, 4-chlorophenylalanine, 4-carboxyphenylalanine, b-phenylserine b-hydroxyphenylalanine, phenylglycine, a-naphthylalanine, cyclohexylalanine, cyclohexylglycine, indoline-2-carboxylic acid, 1,2,3,4-tetrahydroisoquinoline-3-carboxylic acid, aminomalonic acid, arminomalonic acid ronoamide, N‘-benzyl-N’-methyl-lysine, N′,N′-dibenzyl-lysine, 6-hydroxylysine, ornithine, a-aminocyclopentane carboxylic acid, a-aminocyclohexane carboxylic acid, a-aminocycloheptane carboxylic acid, a-(2-amino-2-norbornane)-carboxylic acid, a,g-diaminobutyric acid, a,b-diaminopropionic acid, hornophenylalanine, and a-tert-butylglycine.

In some embodiments, CAR constructs (including functional portions and functional variants) can be glycosylated, anridated, carboxylated, phosphorylated, esterified, N-acylated, cyclized via, e.g., a disulfide bridge, or converted into an acid addition salt and/or optionally dimerized or polymerized, or conjugated.

In some embodiments, CAR constructs (including functional portions and functional variants thereof) can be obtained by methods known in the art. In some embodiments, CAR constructs may be made by any suitable method of making polypeptides or proteins, including de novo synthesis. CAR constructs can be recombinantly produced using the nucleic acids described herein using standard recombinant methods. See, for instance, Green et al., Molecular Cloning: A Laboratory Manual, 4th ed., Cold Spring Harbor Press, Cold Spring Harbor, NY 2012. Further, portions of some of the CAR constructs described herein (including functional portions and functional variants thereof) can be isolated and/or purified from a source, such as a plant, a bacterium, an insect, a mammal, e.g., a rat, a human, etc. Methods of isolation and purification are well known in the art. Alternatively, the CAR constructs described herein (including functional portions and functional variants thereof) can be commercially synthesized by companies, such as Synpep (Dublin, CA), Peptide Technologies Corp. (Gaithersburg, MD), and Multiple Peptide Systems (San Diego, CA). In this respect, the CAR constructs can be synthetic, recombinant, isolated, and/or purified.

Further provided herein are nucleic acids comprising a nucleotide sequence encoding any of the CAR constructs described herein (including functional portions and functional variants thereof). The nucleic acids described herein may comprise a nucleotide sequence encoding any of the leader sequences (e.g., signal peptides), antigen binding domains, transmembrane domains, linker regions, costimulatory signaling domains, and/or intracellular T cell signaling domains described herein.

In some aspects, any of the antigen-binding domains described herein may be operably linked to another domain of the CAR, such as the transmembrane domain or the intracellular domain, for expression in the cell. In some embodiments, a nucleic acid encoding the antigen-binding domain is operably linked to a nucleic acid encoding a transmembrane domain and a nucleic acid encoding an intracellular domain.

In some embodiments, a nucleic acid encoding the anti-EMR2 antigen binding domain is operably linked to a nucleic acid encoding a linker region, a nucleic acid encoding a transmembrane domain, and/or a nucleic acid encoding an intracellular domain (e.g., a costimulatory signaling domain, a signaling domain). In some embodiments, the CAR comprises any of the anti-EMR2 antibodies or antigen-binding fragments thereof, described herein (e.g., comprising one or more CDRs described herein). In some embodiments, the CAR comprises any of the anti-EMR2 antibodies or antigen-binding fragments thereof, provided in any one of SEQ ID NOs: 65, 71, 73, 75, 81, 83, 89, 91, 93, 99, 101.

In some embodiments, the CAR comprises a linker region. In some embodiments, the light chain variable region and the heavy chain variable region of the antigen-binding domain can be joined to each other by a linker. In some embodiments, the antigen-binding domain can be joined to another domain, such as a transmembrane domain, hinge, and/or intracellular domain with a linker region. The linker may comprise any suitable amino acid sequence. In some embodiments, the linker is a Gly/Ser linker from about 1 to about 100, from about 3 to about 20, from about 5 to about 30, from about 5 to about 18, or from about 3 to about 8 amino acids in length and consists of glycine and/or serine residues in sequence. Accordingly, the Gly/Ser linker may consist of glycine and/or serine residues. Preferably, the Gly/Ser linker comprises the amino acid sequence of GGGGS (SEQ ID NO: 1), and multiple amino acid sequences of SEQ ID NO: 1 may be present within the linker. Any linker sequence may be used as a spacer between the antigen-binding domain and any other domain of the CAR, such as the transmembrane domain. In some, embodiments, the region linker is ([G]x[S]y)z, for example wherein x can be 1-10, y can be 1-3, and z can be 1-5. In some embodiments, the linker region comprises the amino acid sequence GGGGSGGGGS (SEQ ID NO: 2). In some embodiments, the linker region comprises the amino acid sequence GGGGSGGGGSGGGGS (SEQ ID NO: 3). In some embodiments, the linker region comprises an amino acid sequence of any one of SEQ ID NOs: 1-3.

In some embodiments, the antigen-binding domain comprises one or more leader sequences (signal peptides, signal sequence), such as those described herein. In some embodiments, the leader sequence may be positioned at the amino terminus of the CAR within the CAR construct. The leader sequence may comprise any suitable leader sequence, e.g., any CARs described herein may comprise any leader sequence, such as those described herein. In some embodiments, while the leader sequence may facilitate expression of the released CARs on the surface of the cell, the presence of the leader sequence in an expressed CAR is not necessary in order for the CAR to function. In some embodiments, upon expression of the CAR on the cell surface, the leader sequence may be cleaved off. Accordingly, in some embodiments, the released CARs (e.g., surface expressed) lack a leader sequence. In some embodiments, the CARs within the CAR construct lack a leader sequence.

### Hinge

In some embodiments, the CAR comprises a hinge/spacer region that links the extracellular antigen-binding domain to another domain, such as a transmembrane domain. The hinge/spacer region can be flexible enough to allow the antigen-binding domain to orient in different directions to facilitate target antigen recognition. In some embodiments, the hinge domain is a portion of the hinge domain of CD8a or CD28, e.g., a fragment containing at least 15 (e.g., 20, 25, 30, 35, or 40) consecutive amino acids of the hinge domain of CD8a or CD28.

In some embodiments, the CAR comprises a hinge domain, such as a hinge domain from CD8, CD28, or IgG4. In some embodiments, the hinge domain is a CD8 (e.g., CD8a) hinge domain. In some embodiments, the CD8 hinge domain is human (e.g., obtained from/derived from a human protein sequence). In some embodiments, the CD8 hinge domain comprises, consists of, or consists essentially of SEQ ID NO: 4.

CD8 hinge region
[SEQ ID NO: 4]
TTTPAPRPPTPAPTIASQPLSLRPEACRPAAGGAVHTRGLDFACD

In some embodiments, the hinge domain is a CD28 hinge domain. In some embodiments, the CD28 hinge domain is human (e.g., obtained from/derived from a human protein sequence). In some embodiments, the CD28 hinge domain comprises, consists of, or consists essentially of SEQ ID NO: 5.

CD28 hinge region
[SEQ ID NO: 5]
AAAIEVMYPPPYLDNEKSNGTIIHVKGKHLCPSPLFPGPSKP

Hinge domains of antibodies, such as an IgG, IgA, IgM, IgE, or IgD antibody, are also compatible for use in the chimeric receptors described herein. In some embodiments, the hinge domain is the hinge domain that joins the constant domains CH1 and CH2 of an antibody. In some embodiments, the hinge domain is of an antibody and comprises the hinge domain of the antibody and one or more constant regions of the antibody. In some embodiments, the hinge domain comprises the hinge domain of an antibody and the CH3 constant region of the antibody. In some embodiments, the hinge domain comprises the hinge domain of an antibody and the CH2 and CH3 constant regions of the antibody. In some embodiments, the antibody is an IgG, IgA, IgM, IgE, or IgD antibody. In some embodiments, the antibody is an IgG antibody. In some embodiments, the antibody is an IgG1, IgG2, IgG3, or IgG4 antibody. In some embodiments, the hinge region comprises the hinge region and the CH2 and CH3 constant regions of an IgG1 antibody. In some embodiments, the hinge region comprises the hinge region and the CH3 constant region of an IgG1 antibody. In some embodiments, the hinge domain is an IgG4 hinge domain.

Also within the scope of the present disclosure are CARs comprising a hinge domain that is a non-naturally occurring peptide. In some embodiments, the hinge domain between the C-terminus of the extracellular ligand-binding domain of an Fc receptor and the N-terminus of the transmembrane domain is a peptide linker, such as a (GlyxSer)ₙ linker, wherein x and n, independently can be an integer between 3 and 12, including 3, 4, 5, 6, 7, 8, 9, 10, 11, 12, or more. In some embodiments, the linker region comprises an amino acid sequence of any one of SEQ ID NOs: 1-3.

Additional peptide linkers that may be used in a hinge domain of the chimeric receptors described herein are known in the art. See, e.g., Wriggers et al. Current Trends in Peptide Science (2005) 80(6): 736-74 and PCT Publication No. WO 2012/088461.

In some embodiments, the hinge/spacer region of a presently disclosed CAR comprises a native or modified hinge region of a CD28 polypeptide as described herein. In certain embodiments, the hinge/spacer region of a presently disclosed CAR construct comprises a native or modified hinge region of a CD8a polypeptide as described herein. In some embodiments, the hinge/spacer region of any of the disclosed CAR constructs comprises a native or modified hinge region of a IgG4 polypeptide as described herein.

### Transmembrane Domain

With respect to the transmembrane domain, a CAR can be designed to comprise a transmembrane domain that connects the antigen-binding domain of the CAR to an intracellular region of the CAR. In some embodiments, the transmembrane domain is naturally associated with one or more of the domains in the CAR. In some instances, the transmembrane domain can be selected or modified by amino acid substitution to avoid binding of such domains to the transmembrane domains of the same or different surface membrane proteins to minimize interactions with other members of the receptor complex.

The transmembrane domain may be derived either from a natural or from a synthetic source. Where the source is natural, the domain may be derived from any membrane-bound or transmembrane protein. Transmembrane regions of particular use in this invention may be derived from (i.e., comprise at least the transmembrane region(s) of the alpha, beta or zeta chain of the T-cell receptor, CD28, CD3 epsilon, CD45, CD4, CD5, CD8, CD8a, CD9, CD 16, CD22, CD33, CD37, CD64, CD80, CD86, CD134, CD137, CD154, Toll-like receptor 1 (TLR1), TLR2, TLR3, TLR4, TLR5, TLR6, TLR7, TLR8, and TLR9.

In some embodiments, the transmembrane domain may be synthetic, in which case it will comprise predominantly hydrophobic residues such as leucine and valine. Preferably a triplet of phenylalanine, tryptophan and valine will be found at each end of a synthetic transmembrane domain.

In some embodiments, the transmembrane domain is a CD8 (e.g., CD8a) transmembrane domain. In some embodiments, the CD8 transmembrane domain is human (e.g., obtained from/derived from a human protein sequence). In some embodiments, a CD8 transmembrane domain comprises, consists of, or consists essentially of SEQ ID NO: 6.

CD8 transmembrane region
[SEQ ID NO: 6]
IYIWAPLAGTCGVLLLSLVITLYC

In some embodiments, the transmembrane domain is a CD28 transmembrane domain. In some embodiments, the CD28 transmembrane domain is human (e.g., obtained from/derived from a human protein sequence). In some embodiments, the CD28 transmembrane domain comprises, consists of, or consists essentially of SEQ ID NO: 7.

CD28 transmembrane domain
[SEQ ID NO: 7]
FWVLVVVGGVLACYSLLVTVAFIIFWVRSKRSRLLHSDYMNMTPRRPGP
TRKHYQPYAPPRDFAAYRS

### Intracellular Signaling Domains

In some embodiments, the CAR construct comprises an intracellular signaling domain, which may be comprised of one or more signaling domains and costimulatory domains. The intracellular signaling domain of the CAR, is involved in activation of the cell in which the CAR is expressed. In some embodiments, the intracellular signaling domain of the CAR construct described herein is involved in activation of a T lymphocyte or NK cells. In some embodiments, the signaling domain of the CAR construct described herein includes a domain involved in signal activation and/or transduction.

Examples of an intracellular signaling domains for use in the CAR constructs described herein include, but are not limited to, the cytoplasmic portion of a surface receptor, co-stimulatory molecule, and any molecule that acts in concert to initiate signal transduction in a cell (e.g., an immune cell (e.g., a T lymphocyte), NK cell), as well as any derivative or variant of these elements and any synthetic sequence that has the same functional capability.

Examples of the signaling domains that may be used in the intracellular signaling domain of the CARs described herein include, without limitation, a fragment or domain from one or more molecules or receptors including, but are not limited to, TCR, CD3 zeta (CD3ξ), CD3 gamma, CD3 delta, CD3 epsilon, CD86, common FcR gamma, FcR beta (Fc Epsilon Rib), CD79a, CD79b, Fcgamma RIIa, DAP10, DAP 12, T cell receptor (TCR), CD27, CD28, 4-1BB (CD137), OX40, CD30, CD40, PD-1, ICOS, lymphocyte function-associated antigen-1 (LFA-1), CD2, CD7, LIGHT, NKG2C, B7-H3, a ligand that specifically binds with CD83, CDS, ICAM-1, GITR, BAFFR, HVEM (LIGHTR), SLAMF7, NKp80 (KLRFI), CD127, CD160, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD 1 id, ITGAE, CD103, ITGAL, CDiia, LFA-1, ITGAM, CDiib, ITGAX, CDiic, ITGB1, CD29, ITGB2, CD18, LFA-1, ITGB7, TNFR2, TRANCE/RANKL, DNAM1 (CD226), SLAMF4 (CD244, 2B4), CD84, CD96 (Tactile), CEACAMl, CRTAM, Ly9 (CD229), CD160 (BY55), PSGL1, CD100 (SEMA4D), CD69, SLAMF6 (NTB-A, Ly108), SLAM (SLAMFI, CD150, IPO-3), BLAME (SLAMF8), SELPLG (CD 162), LTBR, LAT, GADS, SLP-76, PAG/Cbp, NKp44, NKp30, NKp46, NKG2D, Toll-like receptor 1 (TLR1), TLR2, TLR3, TLR4, TLR5, TLR6, TLR7, TLR8, TLR9, other co-stimulatory molecules described herein, any derivative, variant, or fragment thereof, any synthetic sequence of a co-stimulatory molecule that has the same functional capability, and any combination thereof.

Any cytoplasmic signaling domain can be used in the CARs described herein. In general, a cytoplasmic signaling domain relays a signal, such as interaction of an extracellular ligand-binding domain with its ligand, to stimulate a cellular response, such as inducing an effector function of the cell (e.g., cytotoxicity).

As will be evident to one of ordinary skill in the art, a factor involved in T cell activation is the phosphorylation of immunoreceptor tyrosine-based activation motif (ITAM) of a cytoplasmic signaling domain. Any ITAM-containing domain known in the art may be used to construct the chimeric receptors described herein, and included as part of the cytoplasmic signaling domain. In general, an ITAM motif may comprise two repeats of the amino acid sequence YxxL/I separated by 6-8 amino acids, wherein each x is independently any amino acid, producing the conserved motif YxxL/Ix(6-8)YxxL/I. In some embodiments, the cytoplasmic signaling domain is from CD3ξ.

CD3ξ associates with TCRs to produce a signal and contains immunoreceptor tyrosine-based activation motifs (ITAMs). In some embodiments, a CD3ξ intracellular T cell signaling sequence is human (e.g., obtained from or derived from a human protein). In some embodiments, a CD3ξ intracellular T cell signaling sequence comprises, consists of, or consists essentially of the amino acid sequence of SEQ ID NO: 8 or 9, or a sequence that is at least 70%, at least 75%, at least 80%, at least 85%, at least 90%, at least 95%, or at least 99% identical the amino acid sequence of SEQ ID NO: 8 or 9. In some embodiments, an intracellular T cell signaling domain comprises a CD3ξ that contains on or more mutated and/or deleted ITAMs.

CD3 ζ signaling domain (variant A)
[SEQ ID NO: 8]
RVKFSRSADAPAYKQGQNQLYNELNLGRREEYDVLDKRRGRDPEMGGKP
RRKNPQEGLYNELQKDKMAEAYSEIGMKGERRRGKGHDGLYQGLSTATK
DTYDALHMQALPPR
CD3 ζ signaling domain (variant B)
[SEQ ID NO: 9]
RVKFSRSADAPAYQQGQNQLYNELNLGRREEYDVLDKRRGRDPEMGGKP
RRKNPQEGLYNELQKDKMAEAYSEIGMKGERRRGKGHDGLYQGLSTATK
DTYDALHMQALPPR

In certain non-limiting embodiments, an intracellular signaling domain of the CAR further comprises at least one (e.g., 1, 2, 3 or more) co-stimulatory signaling domain. In some embodiments, the co-stimulatory signaling domain comprises at least one co-stimulatory molecule, which can provide optimal lymphocyte activation. In general, many immune cells require co-stimulation, in addition to stimulation of an antigen-specific signal, to promote cell proliferation, differentiation and survival, and to activate effector functions of the cell. Activation of a co-stimulatory signaling domain in a host cell (e.g., an immune cell) may induce the cell to increase or decrease the production and secretion of cytokines, phagocytic properties, proliferation, differentiation, survival, and/or cytotoxicity. The co-stimulatory signaling domain of any co-stimulatory protein may be compatible for use in the chimeric receptors described herein. The type(s) of co-stimulatory signaling domains may be selected based on factors such as the type of the cells in which the CARs would be expressed (e.g., primary T cells, T cell lines, NK cell lines) and the desired immune effector function (e.g., cytotoxicity).

Examples of such co-stimulatory signaling domains include a fragment or domain from one or more molecules or receptors including, without limitation, 4-1BB, CD28, ICOS, TLR1, TLR2, TLR3, TLR4, TLR5, TLR6, TLR7, TLR8, TLR9, TLR10, TLR11, CD116 receptor beta chain, CSF1-R, LRP1/CD91, SR-A1, SR-A2, MARCO, SR-CL1, SR-CL2, SR-C, SR-E, CR1, CR3, CR4, dectin 1, DEC-205, DC-SIGN, CD14, CD36, LOX-1, CD11b, together with any of the signaling domains listed in the above paragraph in any combination. In some embodiments, the intracellular signaling domain of the CAR includes any portion of one or more co-stimulatory signaling molecules, such as at least one signaling domain from CD3, Fc epsilon RI gamma chain, any derivative or variant thereof, including any synthetic sequence thereof that has the same functional capability, and any combination thereof.

In some embodiments, one or more co-stimulatory signaling domains (e.g., 1, 2, 3, or more) are included in a CAR construct with a CD3ξ intracellular T cell signaling sequence. In some embodiments, the one or more co-stimulatory signaling domains are selected from CD137 (4-1BB) and CD28, or a combination thereof. In some embodiments, the CAR comprises a 4-1BB (CD137) costimulatory signaling domain. In some embodiments, the CAR comprises a CD28 costimulatory signaling domain. In some embodiments, the CAR comprises both a 4-1BB costimulatory signaling domain and a CD28 costimulatory signaling domain.

4-1BB, also known as CD137, transmits a potent costimulatory signal to T cells, promoting differentiation and enhancing long-term survival of T lymphocytes. In some embodiments, a 4-1BB intracellular signaling sequence is human (e.g., obtained from/derived from a human protein sequence). In some embodiments, the 4-1BB intracellular T cell signaling sequence comprises, consists of, or consists essentially of the amino acid sequence of SEQ ID NO: 10. In some embodiments, the 4-1BB costimulatory signaling domain comprises, consists of, or consists essentially of the amino acid sequence of SEQ ID NO: 10, or a sequence that is at least 70%, at least 75%, at least 80%, at least 85%, at least 90%, at least 95%, or at least 99% identical the amino acid sequence of SEQ ID NO: 10.

4-1BB costimulatory signaling domain
[SEQ ID NO: 10]
KRGRKKLLYIFKQPFMRPVQTTQEEDGCSCRFPEEEEGGCEL

Some suitable costimulatory domains are provided herein, and other suitable costimulatory domains and costimulatory domain sequences will be apparent to the skilled artisan based on the present disclosure in view of the knowledge in the art. Suitable costimulatory domains include, for example, those described in Weinkove et al., Selecting costimulatory domains for chimeric antigen receptors: functional and clinical considerations, Clin Transl Immunology (2019) 8(5): e1049, the entire contents of which are incorporated herein by reference.

Between the antigen-binding domain and the transmembrane domain of the CAR, or between the intracellular signaling domain and the transmembrane domain of the CAR, a spacer domain may be incorporated. As used herein, the term “spacer domain” generally means any oligo- or polypeptide that functions to link the transmembrane domain to, either the antigen binding domain or, the intracellular domain in the polypeptide chain. In some embodiments, the spacer domain may comprise up to 300 amino acids, preferably 10 to 100 amino acids and most preferably 25 to 50 amino acids. In some embodiments, a short oligo- or polypeptide linker, preferably between 2 and 10 amino acids in length may form the linkage between the transmembrane domain and the intracellular domain of the CAR. An example of a linker includes a glycine-serine doublet.

### Signal Peptides

In some embodiments, any of the CARs described herein may further comprise a signal peptide (signal sequence). In general, signal peptides are short amino acid sequences that target a polypeptide to a site in a cell. In some embodiments, the signal peptide directs the CAR to the secretory pathway of the cell and will allow for integration and anchoring of the CAR into the lipid bilayer at the cell surface. Signal sequences including signal sequences of naturally occurring proteins or synthetic, non-naturally occurring signal sequences, that are compatible for use in the chimeric receptors described herein will be evident to one of skill in the art.

The CARs described herein may be prepared in constructs with, e.g., self-cleaving peptides, such that the CAR constructs containing anti-EMR2 CAR components are bicistronic, tricistronic, etc.

Various CAR constructs and numerous elements of CAR constructs (for example, various EMR2 binding domains, signal peptides, linkers, hinge sequences, transmembrane domains, costimulatory domains, and signaling domains) are disclosed herein, and those of skill in the art will be able to ascertain the sequences of these elements and of additional suitable elements known in the art based on the present disclosure in view of the knowledge in the art. Exemplary CAR element sequences, e.g., for EMR2 binding domains, signal peptides, linkers, hinge sequences, transmembrane domains, costimulatory domains, and signaling domains, are disclosed, for example, in PCT Publication No. WO2016/120218, e.g., throughout the specification and in Tables 1-8, the entire contents of which are incorporated herein by reference.

Any of the anti-EMR2 antibodies, or antigen-binding fragments thereof, may be used in a CAR construct, with any one or more of the additional components described herein, e.g., hinge, transmembrane domain, co-stimulatory domain, intracellular signaling domain. In some embodiments, the CAR construct comprises an EMR2 binding domain comprising one or more of the CDR sequences provided by DSL, DGL, or SEQ ID NOs: 12-28 and 30-58, a CD8a transmembrane domain, a CD8a hinge domain, a CD137 (4-1BB) co-stimulatory domain, and a CD3ξ intracellular signaling domain. In some embodiments, the CAR construct comprises an EMR2 binding domain comprising three of the CDR sequences provided by DSL, DGL, or by SEQ ID NOs: 12-28 and 30-58 (e.g., a VH), a CD8a transmembrane domain, a CD8a hinge domain, a CD137 (4-1BB) co-stimulatory domain, and a CD3ξ intracellular signaling domain. In some embodiments, the CAR construct comprises an EMR2 binding domain comprising six of the CDR sequences provided by DSL, DGL, or by SEQ ID NOs: 12-28 and 30-58 (e.g., a scFv), a CD8a transmembrane domain, a CD8a hinge domain, a CD137 (4-1BB) co-stimulatory domain, and a CD3ξ intracellular signaling domain.

As will be understood by one of ordinary skill in the art, one or more of the domains of the exemplary CAR constructs may be replaced by another domain. For example, the exemplary CAR constructs provided below comprise a CD8a transmembrane domain, a CD8a hinge domain, a CD137 (4-1BB) co-stimulatory domain, and a CD3ξ intracellular signaling domain, any one of the domains (such as the hinge domain, transmembrane domain, co-stimulatory domain, and/or intracellular signaling domain) may be replaced by another domain, such as those described herein.

### CAR Clones

An exemplary CAR construct, as described herein, comprises a nucleotide sequence encoding an EMR2 binding domain comprising any one of SEQ ID NOs: 64, 70, 72, 74, 80, 82, 88, 90, 92, 98, and 100, a CD8a transmembrane domain, a CD8a hinge domain, a CD137 (4-1BB) co-stimulatory domain, and a CD3ξ intracellular signaling domain.

In some embodiments, a CAR construct comprising any of the EMR2 binding domains described herein is encoded in a recombinant expression vector. In some embodiments, the recombinant expression vector includes a promoter (e.g., an SFFV promoter, an EFla promoter, a tEFIa promoter, a hPGK promoter, a SFFV promoter, or a MND promoter).

Any of the fusion proteins, such as any of the CARs described herein may be expressed in a cell and thereby presented on the surface of the cell. In some embodiments, the cell may be an immune cell, such as a T cell (i.e., T lymphocyte) or an NK cell. A T cell lymphocyte can be any T cell, such as a cultured T cell, e.g., a primary T cell, or a T cell from a cultured T cell line, e.g., TIB-153™, Jurkat, SupTl, etc., or a T cell obtained from a mammal.

### Nucleotide Sequences and Expression

The present disclosure includes nucleotide sequences encoding any one or more anti-EMR2 antibodies described herein (e.g., a VH described herein), or portion thereof (e.g., one or more CDRs described herein), and/or one or more fusion proteins described herein. In various instances, such nucleotide sequences may be present in a vector, such as an expression vector. In various instances such nucleotides may be present in the genome of a cell, e.g., a cell of a subject in need of treatment or a cell for production of an antibody, e.g., a mammalian cell for production of an antibody.

In some embodiments, any of the antibodies described herein are encoded by a polynucleotide comprised in a vector, e.g., a viral vector. Optionally, a polynucleotide encoding a polypeptide as described herein can be codon-optimized to enhance expression or stability. Codon optimization may be performed according to any standard methods known in the art. In some embodiments, expression of the polypeptide can be driven by a constitutively expressed promoter or an inducibly expressed promoter. In some embodiments, an antibody as described herein includes a signal peptide. Signal peptides can be derived from any protein that has an extracellular domain or is secreted. An antibody as described herein may include any signal peptides known in the art.

Retroviruses, such as lentiviruses, provide a convenient platform for delivery of nucleic acid sequences encoding a gene, or chimeric gene of interest. A selected nucleic acid sequence can be inserted into a vector and packaged in retroviral particles using techniques known in the art. The recombinant virus can then be isolated and delivered to cells, e.g., in vitro or ex vivo. Retroviral systems are well known in the art and are described in, for example, U.S. Pat. No. 5,219,740; Kurth and Bannert (2010) “Retroviruses: Molecular Biology, Genomics and Pathogenesis” Calster Academic Press (ISBN: 978-1-90455-55-4); and Hu and Pathak Pharmacological Reviews (2000) 52:493-512; which are incorporated by reference herein in their entirety. In some embodiments, an antibody described herein is expressed in a mammalian cell via transfection or electroporation of an expression vector comprising nucleic acid encoding the antibody. Transfection or electroporation methods are known in the art.

In another aspect, the disclosure relates to a cell, e.g., a mammalian cell, comprising any of the antibodies described herein; or a nucleic acid encoding any of the antibodies described herein. In one embodiment, the cell comprises an antibody described herein, or a nucleic acid encoding such an antibody described herein. The cell or tissue, e.g., mammalian cell or tissue, can be of human, primate, hamster, rabbit, rodent, cow, pig, sheep, horse, goat, dog or cat origin. In some embodiments, any other mammalian cell may be used. In some embodiments, the mammalian cell is human.

Efficient expression of an antibody described herein can be assessed using standard assays that detect the mRNA, DNA, or a gene product of the nucleic acid encoding the antibody, such as RT-PCR, FACS, Northern blotting, Western blotting, ELISA, flow cytometry, or immunohistochemistry. In some embodiments, the antibody described herein is encoded by recombinant nucleic acid sequence.

EMR2-Associated Diseases and/or Disorders

The present disclosure provides, among other things, compositions and methods for treating a disease associated with expression of EMR2 or a condition associated with cells expressing EMR2, including, e.g., a proliferative disease such as a cancer or malignancy (e.g., a hematopoietic malignancy), or a precancerous condition such as a myelodysplasia, a myelodysplastic syndrome or a preleukemia.

EGF-like module-containing mucin-like hormone receptor-like 2 (EMR2), also known as CD312, is a 823-amino acid, ~90 kDa protein (depending on isoform) of the EGF-seven-span transmembrane (TM7) family of adhesion G protein-coupled receptors (GPCR). EMR2 is highly homologous with CD97 from the same receptor family. EMR2 forms a heterodimer and binds to chondroitin sulfate B via its EGF-like domain 4 to mediate cell adhesion, granulocyte chemotaxis, degranulation, and the release of pro-inflammatory cytokines in macrophages. See, e.g., Kuan-Yu et al. Front. Immunol. (2017) 8:373. EMR2 is restricted to myeloid cells including monocytes, macrophages, granulocytes and dendritic cells.

The ADGRE2 gene located on human chromosome 19 encodes human EMR2 and canonically contains 19 exons, although a number of isoforms exist with varying number EGF domains due to alternative RNA splicing. The dominant isoform in whole blood contains 17 exons. See, e.g., Safaee et al. Onc. Rev. (2014). 8(242):20-24.

EMR2 expression has been associated with hematopoietic disorders, i.e., relapsed/refractory acute myeloid leukemia (AML). Analysis of target distribution in AML patient cells indicated that EMR2 is expressed on over 90% of AML cells (including leukemic stem cells) in most AML patients, including a majority of relapsed/refractory AML patient samples, along with CD33, CLL1, and CD123.

In some embodiments, the hematopoietic malignancy or hematological disorder is associated with EMR2 expression. A hematopoietic malignancy has been described as a malignant abnormality involving hematopoietic cells (e.g., blood cells, including progenitor and stem cells). Examples of hematopoietic malignancies include, without limitation, Hodgkin lymphoma, non-Hodgkin lymphoma, leukemia, or multiple myeloma. Exemplary leukemias include, without limitation, acute myeloid leukemia, acute lymphoid leukemia, chronic myelogenous leukemia, acute lymphoblastic leukemia or chronic lymphoblastic leukemia, and chronic lymphoid leukemia.

In some embodiments, cells involved in the hematopoietic malignancy are resistant to conventional or standard therapeutics used to treat the malignancy. For example, the cells (e.g., cancer cells) may be resistant to a chemotherapeutic agent and/or CAR T cells used to treat the malignancy.

In some embodiments, the leukemia is acute myeloid leukemia (AML). Acute myeloid leukemia (AML) is a cancer of the bone marrow that needs more effective therapies. According to the National Cancer Institute, more than 60,000 people in the U.S. have AML, and less than 30% of patients survive five years following diagnosis. AML cells can be characterized and distinguished from other cells by detecting cell surface marker expression. AML cells can be EMR2+, CD33+ (though some are CD33−), CD45+, and CDw52+. AML is characterized as a heterogeneous, clonal, neoplastic disease that originates from transformed cells that have progressively acquired critical genetic changes that disrupt key differentiation and growth-regulatory pathways. See, e.g., Dohner et al., NEJM, (2015) 373:1136. Without wishing to be bound by theory, it is believed in some embodiments, that EMR2 is expressed on myeloid leukemia cells as well as on normal myeloid and monocytic precursors and is an attractive target for AML therapy.

In some embodiments, the hematopoietic malignancy or hematological disease/disorder associated with EMR2 is a precancerous condition such as a myelodysplasia, a myelodysplastic syndrome or a preleukemia. Myelodysplastic syndromes (MDS) are hematological medical conditions characterized by disorderly and ineffective hematopoiesis, or blood production. Thus, the number and quality of blood-forming cells decline irreversibly. Some patients with MDS can develop severe anemia, while others are asymptomatic. The classification scheme for MDS is known in the art, with criteria designating the ratio or frequency of particular blood cell types, e.g., myeloblasts, monocytes, and red cell precursors. MDS includes refractory anemia, refractory anemia with ring sideroblasts, refractory anemia with excess blasts, refractory anemia with excess blasts in transformation, chronic myelomonocytic leukemia (CML). In some embodiments, MDS can progress to AML.

Additionally, aberrant EMR2 expression has been associated with human breast carcinoma and patient survival. See, e.g., Davies et al. Oncol. Rep. (2011) 25(3): 619-627. EMR2 overexpression has also been associated with other cancers including bladder carcinoma, colorectal carcinoma, gastric and esophageal carcinoma, and glioblastoma. See, e.g., Safaee et al. Oncol. Rev. (2014) 8(242): 20-24.

Due to the shared expression of EMR2 on both activated healthy cells as well as being an expressed antigen on malignant cells, therapeutic targeting of EMR2 may result in killing of heathy cells.

In various instances, an antibody and/or fusion protein and/or cells expressing any of the foregoing described herein treats, alleviates, reduces the prevalence of, reduces the frequency of, or reduces the level or amount of one or more symptoms or biomarkers of an EMR2-associated disorder (e.g., AML, MDS). Specific symptoms and progression of symptoms vary among subjects. Thus, in some embodiments, an antibody and/or fusion protein described herein is administered to a subject in need thereof, e.g., a subject having an EMR2-associated disorder (e.g., AML, MDS). In some embodiments, administration of any of the antibodies and/or fusion proteins, including CARs or cells expressing the CARs, described herein prevents cancer or hematopoietic malignancy or pre-malignancy, including reducing one or more symptoms and/or delaying the progression of the disease.

In some cases, a subject may initially respond to a therapy (e.g., for a hematopoietic malignancy) and subsequently experience relapse. In some embodiments, the subject has or is susceptible to relapse of a hematopoietic malignancy (e.g., AML) following administration of one or more previous therapies. In some embodiments, the administration of any of the antibodies and/or fusion proteins described herein reduce the subject's risk of relapse or the severity of relapse.

In some embodiments, one or more of the anti-EMR2 antibodies described herein are used in a method of treating one or more disorders described herein, e.g., one or more diseases or disorders associated with EMR2 expression. In some embodiments, the method comprises administering to a subject in need thereof a therapeutically effective amount of an antibody, or antigen-binding fragment thereof, described herein, fusion proteins, or a cell expressing any of the foregoing. In some embodiments, one or more of the anti-EMR2 antibodies described herein, including fusion proteins comprising any of the anti-EMR2 antibodies, are used in a method of treating diseases or disorders associated with EMR2 expression. In some embodiments, one or more of the anti-EMR2 antibodies described herein, including fusion proteins comprising any of the anti-EMR2 antibodies, or a cell expressing any of the foregoing, are used in a method of treating neoplastic diseases and malignancies of the blood that are associated with EMR2 expression. In some embodiments, one or more anti-EMR2 antibodies described herein, including fusion proteins comprising any of the anti-EMR2 antibodies, or a cell expressing any of the foregoing, are used in a method of treating MDS or AML.

In various instances, administration of an antibody and/or fusion protein described herein or a cell expressing any of the foregoing results in a decrease in the prevalence, frequency, level, and/or amount of one or more symptoms or biomarkers of an EMR2-associated disorder, e.g., a decrease of at least 3%, 4%, 5%, 6%, 7%, 8%, 9%, 10%, 15%, 20%, 25%, 30%, 35%, 40%, 45%, 50%, 60%, 70%, 80%, 90%, 95%, 99%, or 100% of one or more symptoms or biomarkers as compared to a prior measurement in the subject or to a reference value.

In some embodiments, an antibody and/or fusion protein described herein or a cell expressing any of the foregoing can be used in a number of diagnostic and/or therapeutic applications. For example, detectably-labeled versions of antibodies as described herein can be used in assays to detect the presence or amount of EMR2 in a sample (e.g., a biological sample). Antibodies and/or fusion proteins described herein or a cell expressing any of the foregoing can be used in in vitro assays for studying inhibition of EMR2 activity. In some embodiments, an antibody and/or fusion protein described herein or a cell expressing any of the foregoing can be used as a positive control in an assay designed to identify additional novel compounds that inhibit EMR2 or otherwise are useful for treating a EMR2-associated disorder.

Antibodies and/or fusion proteins described herein or a cell expressing any of the foregoing may be used in monitoring a subject, e.g., a subject having, suspected of having, at risk of developing, or under treatment for one or more EMR2-associated disorders. Monitoring may include determining the amount or activity of EMR2 in a subject, e.g., in the serum of a subject. In some embodiments, the evaluation is performed at least one (1) hour, e.g., at least 2, 4, 6, 8, 12, 24, or 48 hours, or at least 1 day, 2 days, 4 days, 10 days, 13 days, 20 days or more, or at least 1 week, 2 weeks, 4 weeks, 10 weeks, 13 weeks, 20 weeks or more, after an administration of an antibody and/or fusion protein as described herein or a cell expressing any of the foregoing. The subject can be evaluated in one or more of the following periods: prior to beginning of treatment; during the treatment; or after one or more elements of the treatment have been administered. Evaluation can include evaluating the need for further treatment, e.g., evaluating whether a dosage, frequency of administration, or duration of treatment should be altered. It can also include evaluating the need to add or drop a selected therapeutic modality, e.g., adding or dropping any of the treatments for a EMR2-associated disorder described herein.

### Measuring Interactions of Antibodies and EMR2

The binding properties of an antibody described herein to EMR2 can be measured by methods known in the art, e.g., one of the following methods: BIACORE™ analysis, enzyme-linked immunosorbent assay (ELISA), x-ray crystallography, sequence analysis and scanning mutagenesis. The binding interaction of an antibody and EMR2 can be analyzed using surface plasmon resonance (SPR). SPR or Biomolecular Interaction Analysis (BIA) detects bio-specific interactions in real time, without labeling any of the interactants. Changes in the mass at the binding surface (indicative of a binding event) of the BIA chip result in alterations of the refractive index of light near the surface. The changes in the refractivity generate a detectable signal, which are measured as an indication of real-time reactions between biological molecules. Methods for using SPR are described, for example, in U.S. Pat. No. 5,641,640; Raether (1988) Surface Plasmons Springer Verlag; Sjolander and Urbaniczky (1991) Anal. Chem. 63:2338-2345; Szabo et al. (1995) Curr Opin. Struct. Biol. 5:699-705 and on-line resources provided by BIAcore International AB (Uppsala, Sweden). Additionally, a KinExA® (Kinetic Exclusion Assay) assay, available from Sapidyne Instruments (Boise, Id.), can also be used.

Information from SPR can be used to provide an accurate and quantitative measure of the equilibrium dissociation constant (K_{D}), and kinetic parameters, including Koₙ and Ko_{f}f, for the binding of an antibody to EMR2. Such data can be used to compare different molecules. Information from SPR can also be used to develop structure-activity relationships (SAR). Variant amino acids at given positions can be identified that correlate with particular binding parameters, e.g., high affinity.

In certain embodiments, an antibody described herein exhibits high affinity for binding EMR2. In various embodiments, K_{D} of an antibody as described herein for EMR2 is less than about 10⁻⁴, 10⁻⁵, 10⁻⁶, 10⁻⁷, 10⁻⁸, 10⁻⁹, 10⁻¹⁰, 10⁻¹¹, 10⁻¹², 10⁻¹³, 10⁻¹⁴, or 10⁻¹⁵ M. In certain instances, K_{D} of an antibody as described herein for EMR2 is between 0.001 and 1 nM, e.g., 0.001 nM, 0.005 nM, 0.01 nM, 0.05 nM, 0.1 nM, 0.5 nM, or 1 nM.

In some embodiments, an antibody described herein binds to a specific epitope of EMR2, e.g., comprising one or more specific amino acids of EMR2. Without wishing to be bound by theory, disruption of the availability of the specific amino acids of an EMR2 epitope of an antibody described herein (e.g., by mutagenesis or due to binding by a competing antibody) may decrease or eliminate binding of the antibody. In some embodiments, multiple anti-EMR2 antibody to EMR2 interactions are desired, anti-EMR2 antibodies may be selected or designed to target non-competing EMR2 epitopes to avoid interfering with one another.

### Formulations and Administration

In various embodiments, any of the antibodies or portion thereof (e.g., one or more CDRs described herein) and/or one or more fusion proteins described herein can be incorporated into a pharmaceutical composition. Such a pharmaceutical composition can be useful, e.g., for the prevention and/or treatment of diseases, e.g., cancers, such as AML, MDS. Pharmaceutical compositions can be formulated by methods known to those skilled in the art (such as described in Remington's Pharmaceutical Sciences, 17th edition, ed. Alfonso R. Gennaro, Mack Publishing Company, Easton, Pa. (1985)).

In some embodiments, a pharmaceutical composition can be formulated to include a pharmaceutically acceptable carrier or excipient. Examples of pharmaceutically acceptable carriers include, without limitation, any and all solvents, dispersion media, coatings, antibacterial and antifungal agents, isotonic and absorption delaying agents, and the like that are physiologically compatible. Compositions of the present invention can include a pharmaceutically acceptable salt, e.g., an acid addition salt or a base addition salt.

In some embodiments, a composition including an antibody as described herein, e.g., a sterile formulation for injection, can be formulated in accordance with conventional pharmaceutical practices using distilled water for injection as a vehicle. For example, physiological saline or an isotonic solution containing glucose and other supplements such as D-sorbitol, D-mannose, D-mannitol, and sodium chloride may be used as an aqueous solution for injection, optionally in combination with a suitable solubilizing agent, such as, for example, an alcohol such as ethanol and/or a polyalcohol such as propylene glycol or polyethylene glycol, and/or a nonionic surfactant such as polysorbate 80 (TWEEN-80™) or HCO-50.

As disclosed herein, a pharmaceutical composition may be in any form known in the art. Such forms include, e.g., liquid, semi-solid and solid dosage forms, such as liquid solutions (e.g., injectable and infusible solutions), dispersions or suspensions, tablets, pills, powders, liposomes and suppositories.

Selection or use of any particular form may depend, in part, on the intended mode of administration and therapeutic application. For example, compositions containing a composition intended for systemic or local delivery can be in the form of injectable or infusible solutions. Accordingly, compositions can be formulated for administration by a parenteral mode (e.g., intravenous, subcutaneous, intraperitoneal, or intramuscular injection). As used herein, parenteral administration refers to modes of administration other than enteral and topical administration, usually by injection, and include, without limitation, intravenous, intranasal, intraocular, pulmonary, intramuscular, intraarterial, intrathecal, intracapsular, intraorbital, intracardiac, intradermal, intrapulmonary, intraperitoneal, transtracheal, subcutaneous, subcuticular, intraarticular, subcapsular, subarachnoid, intraspinal, epidural, intracerebral, intracranial, intracarotid and intrasternal injection and infusion.

Route of administration can be parenteral, for example, administration by injection, transnasal administration, transpulmonary administration, or transcutaneous administration. Administration can be systemic or local by intravenous injection, intramuscular injection, intraperitoneal injection, or subcutaneous injection.

In some embodiments, a pharmaceutical composition of the present invention can be formulated as a solution, microemulsion, dispersion, liposome, or other ordered structure suitable for stable storage at high concentration. Sterile injectable solutions can be prepared by incorporating a composition described herein in the required amount in an appropriate solvent with one or a combination of ingredients enumerated above, as required, followed by filter sterilization. Generally, dispersions are prepared by incorporating a composition described herein into a sterile vehicle that contains a basic dispersion medium and the required other ingredients from those enumerated above. In the case of sterile powders for the preparation of sterile injectable solutions, methods for preparation include vacuum drying and freeze-drying that yield a powder of a composition described herein plus any additional desired ingredient (see below) from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition a reagent that delays absorption, for example, monostearate salts, and gelatin.

A pharmaceutical composition can be administered parenterally in the form of an injectable formulation comprising a sterile solution or suspension in water or another pharmaceutically acceptable liquid. For example, the pharmaceutical composition can be formulated by suitably combining the therapeutic molecule with pharmaceutically acceptable vehicles or media, such as sterile water and physiological saline, vegetable oil, emulsifier, suspension agent, surfactant, stabilizer, flavoring excipient, diluent, vehicle, preservative, binder, followed by mixing in a unit dose form required for generally accepted pharmaceutical practices. The amount of active ingredient included in a pharmaceutical preparation is such that a suitable dose within the designated range is provided. Non-limiting examples of oily liquid include sesame oil and soybean oil and may be combined with benzyl benzoate or benzyl alcohol as a solubilizing agent. Other items that may be included are a buffer such as a phosphate buffer, or sodium acetate buffer, a soothing agent such as procaine hydrochloride, a stabilizer such as benzyl alcohol or phenol, and an antioxidant. A formulated injection can be packaged in a suitable ampule.

In various embodiments, subcutaneous administration can be accomplished by means of a device, such as a syringe, a prefilled syringe, an auto-injector (e.g., disposable or reusable), a pen injector, a patch injector, a wearable injector, an ambulatory syringe infusion pump with subcutaneous infusion sets, or other device for combining with antibody drug for subcutaneous injection.

An injection system of the present disclosure may employ a delivery pen as described in U.S. Pat. No. 5,308,341. Pen devices, most commonly used for self-delivery of insulin to patients with diabetes, are well known in the art. Such devices can comprise at least one injection needle (e.g., a 31 gauge needle of about 5 to 8 mm in length), are typically pre-filled with one or more therapeutic unit doses of a therapeutic solution and are useful for rapidly delivering solution to a subject with as little pain as possible. One medication delivery pen includes a vial holder into which a vial of a therapeutic or other medication may be received. The pen may be an entirely mechanical device or it may be combined with electronic circuitry to accurately set and/or indicate the dosage of medication that is injected into the user. See, e.g., U.S. Pat. No. 6,192,891. In some embodiments, the needle of the pen device is disposable and the kits include one or more disposable replacement needles. Pen devices suitable for delivery of any one of the presently featured compositions are also described in, e.g., U.S. Pat. Nos. 6,277,099; 6,200,296; and 6,146,361, the disclosures of each of which are incorporated herein by reference in their entirety. A microneedle-based pen device is described in, e.g., U.S. Pat. No. 7,556,615, the disclosure of which is incorporated herein by reference in its entirety. See also the Precision Pen Injector (PPI) device, MOLLY™, manufactured by Scandinavian Health Ltd.

In some embodiments, a composition described herein can be therapeutically delivered to a subject by way of local administration. As used herein, “local administration” or “local delivery,” can refer to delivery that does not rely upon transport of the composition or agent to its intended target tissue or site via the vascular system. For example, the composition may be delivered by injection or implantation of the composition or agent or by injection or implantation of a device containing the composition or agent. In certain embodiments, following local administration in the vicinity of a target tissue or site, the composition or agent, or one or more components thereof, may diffuse to an intended target tissue or site that is not the site of administration.

In some embodiments, a composition can be formulated for storage at a temperature below 0 °C (e.g., −20 °C or −80 °C). In some embodiments, the composition can be formulated for storage for up to 2 years (e.g., one month, two months, three months, four months, five months, six months, seven months, eight months, nine months, 10 months, 11 months, 1 year, 11/2 years, or 2 years) at 2-8 °C (e.g., 4 °C). Thus, in some embodiments, the compositions described herein are stable in storage for at least 1 year at 2-8 °C (e.g., 4 °C).

In some embodiments, a pharmaceutical composition can be formulated as a solution. In some embodiments, a composition can be formulated, for example, as a buffered solution at a concentration suitable for storage at 2-8 °C (e.g., 4 °C).

Compositions including one or more antibodies as described herein can be formulated in immunoliposome compositions. Such formulations can be prepared by methods known in the art. Liposomes with enhanced circulation time are disclosed in, e.g., U.S. Pat. No. 5,013,556.

In certain embodiments, compositions can be formulated with a carrier that will protect the compound against rapid release, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are known in the art. See, e.g., J. R. Robinson (1978) “Sustained and Controlled Release Drug Delivery Systems,” Marcel Dekker, Inc., New York.

In some embodiments, administration of an antibody as described herein is achieved by administering to a subject a nucleic acid encoding the antibody. Nucleic acids encoding a therapeutic antibody described herein can be incorporated into a gene construct to be used as a part of a gene therapy protocol to deliver nucleic acids that can be used to express and produce antibody within cells. Expression constructs of such components may be administered in any therapeutically effective carrier, e.g., any formulation or composition capable of effectively delivering the component gene to cells in vivo. Approaches include insertion of the subject gene in viral vectors including recombinant retroviruses, adenovirus, adeno-associated virus, lentivirus, and herpes simplex virus-1 (HSV-1), or recombinant bacterial or eukaryotic plasmids. Viral vectors can transfect cells directly; plasmid DNA can be delivered with the help of, for example, cationic liposomes (lipofectin) or derivatized, polylysine conjugates, gramicidin S, artificial viral envelopes or other such intracellular carriers, as well as direct injection of the gene construct or CaPO₄ precipitation (see, e.g., PCT Publication No. WO 2004/060407). Examples of suitable retroviruses include pLJ, pZIP, pWE and pEM which are known to those skilled in the art (see, e.g., Eglitis et al. Science (19785) 230:1395-1398; Danos and Mulligan Proc Nat Acad Sci USA (1988) 85:6460-6464; Wilson et al. Proc Natl Acad Sci USA (1988) 85:3014-3018; Armentano et al. Proc Natl Acad Sci USA (1990) 87:6141-6145; Huber et al. Proc Natl Acad Sci USA (1991) 88:8039-8043; Ferry et al. Proc Natl Acad Sci USA (1991) 88:8377-8381; Chowdhury et al. Science (1991) 254:1802-1805; van Beusechem et al. Proc Natl Acad Sci USA (1992) 89:7640-7644; Kay et al. Human Gene Therapy (1992)3:641-647; Dai et al. Proc Natl Acad Sci USA (1992) 89:10892-10895; Hwu et al. J Immunol (1993) 150:4104-4115; U.S. Pat. Nos. 4,868,116 and 4,980,286; and PCT Publication Nos. WO89/07136, WO89/02468, WO89/05345, and WO92/07573). Another viral gene delivery system utilizes adenovirus-derived vectors (see, e.g., Berkner et al. BioTechniques (1988) 6:616; Rosenfeld et al. Science (1991) 252:431-434; and Rosenfeld et al. Cell (1992) 68:143-155). Suitable adenoviral vectors derived from the adenovirus strain Ad type 5 d1324 or other strains of adenovirus (e.g., Ad2, Ad3, Ad7, etc.) are known to those skilled in the art. Yet another viral vector system useful for delivery of the subject gene is the adeno-associated virus (AAV). See, e.g., Flotte et al. Am J Respir Cell Mol Biol (1992) 7:349-356; Samulski et al. J Virol (1989) 63:3822-3828; and McLaughlin et al. J Virol (1989) 62:1963-1973.

In some embodiments, the compositions provided herein are present in unit dosage form, which unit dosage form can be suitable for self-administration. Such a unit dosage form may be provided within a container, typically, for example, a vial, cartridge, prefilled syringe or disposable pen. A doser such as the doser device described, for example, in U.S. Pat. No. 6,302,855, may also be used, for example, with an injection system as described herein.

A suitable dose of a composition described herein, which dose is capable of treating or preventing a disorder in a subject, can depend on a variety of factors including, e.g., the age, sex, and weight of a subject to be treated and the particular inhibitor compound used. For example, a different dose of one composition including an antibody, or portion thereof (e.g., one or more CDRs described herein), and/or one or more fusion proteins described herein may be required to treat a subject with a cancer (e.g., AML) as compared to the dose of a different formulation of that antibody. Other factors affecting the dose administered to the subject include, e.g., the type or severity of the disorder. Other factors can include, e.g., other medical disorders concurrently or previously affecting the subject, the general health of the subject, the genetic disposition of the subject, diet, time of administration, rate of excretion, drug combination, and any other additional therapeutics that are administered to the subject. It should also be understood that a specific dosage and treatment regimen for any particular subject can also be adjusted based upon the judgment of the treating medical practitioner.

A pharmaceutical solution can include a therapeutically effective amount of a composition described herein. Such effective amounts can be readily determined by one of ordinary skill in the art based, in part, on the effect of the administered composition, or the combinatorial effect of the composition and one or more additional active agents, if more than one agent is used. A therapeutically effective amount of a composition described herein can also vary according to factors such as the disease state, age, sex, and weight of the individual, and the ability of the composition (and one or more additional active agents) to elicit a desired response in the individual, e.g., amelioration of at least one condition parameter, e.g., amelioration of at least one symptom of a cancer (e.g., AML, MDS). For example, a therapeutically effective amount of a composition described herein can inhibit (lessen the severity of or eliminate the occurrence of) and/or prevent a particular disorder, and/or any one of the symptoms of the particular disorder known in the art or described herein. A therapeutically effective amount is also one in which any toxic or detrimental effects of the composition are outweighed by the therapeutically beneficial effects.

Suitable human doses of any of the compositions described herein can further be evaluated in, e.g., Phase I dose escalation studies. See, e.g., van Gurp et al. Am J Transplantation (2008) 8(8):1711-1718; Hanouska et al. Clin Cancer Res (2007) 13(2, part 1):523-531; and Hetherington et al. Antimicrobial Agents and Chemotherapy (2006) 50(10): 3499-3500.

Toxicity and therapeutic efficacy of compositions can be determined by known pharmaceutical procedures in cell cultures or experimental animals (e.g., animal models of any of the cancers described herein). These procedures can be used, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. A composition described herein that exhibits a high therapeutic index is preferred. While compositions that exhibit toxic side effects may be used, care should be taken to design a delivery system that targets such compounds to the site of affected tissue and to minimize potential damage to normal cells and, thereby, reduce side effects.

Those of skill in the art will appreciate that data obtained from cell culture assays and animal studies can be used in formulating a range of dosage for use in humans. Appropriate dosages of compositions described herein lie generally within a range of circulating concentrations of the compositions that include the ED₅₀ with little or no toxicity. The dosage may vary within this range depending upon the dosage form employed and the route of administration utilized. For a composition described herein, the therapeutically effective dose can be estimated initially from cell culture assays. A dose can be formulated in animal models to achieve a circulating plasma concentration range that includes the IC₅₀ (i.e., the concentration of the antibody which achieves a half-maximal inhibition of symptoms) as determined in cell culture. Such information can be used to more accurately determine useful doses in humans. Levels in plasma may be measured, for example, by high performance liquid chromatography. In some embodiments, e.g., where local administration (e.g., to the eye or a joint) is desired, cell culture or animal modeling can be used to determine a dose required to achieve a therapeutically effective concentration within the local site.

### Combination Therapy

In some embodiments, an anti-EMR2 antibody described herein, or portion thereof (e.g., one or more CDRs described herein), and/or one or more fusion proteins described herein, or a cell expressing any of the foregoing, is administered in combination with one or more additional therapeutic agents, such as a chemotherapeutic agent or an oncolytic therapeutic agent. “Combination therapy,” as used herein, refers to those situations in which two or more different pharmaceutical agents are administered in overlapping regimens so that the subject is simultaneously exposed to both agents. When used in a combination therapy, two or more different agents may be administered simultaneously or separately. Administration in combination can include simultaneous administration of the two or more agents in the same dosage form, simultaneous administration in separate dosage forms, and separate administration. That is, two or more agents can be formulated together in the same dosage form and administered simultaneously. Alternatively, two or more agents can be simultaneously administered, wherein the agents are present in separate formulations. In another alternative, a first agent can be administered just followed by one or more additional agents. In the separate administration protocol, two or more agents may be administered a few minutes apart, or a few hours apart, or a few days apart.

As used herein, the term “chemotherapeutic agent” or “oncolytic therapeutic agent” (e.g., anti-cancer drug, e.g., anti-cancer therapy, e.g., immune cell therapy) has its art-understood meaning referring to one or more pro-apoptotic, cytostatic and/or cytotoxic agents, and/or hormonal agents, for example, specifically including agents utilized and/or recommended for use in treating one or more diseases, disorders or conditions associated with undesirable cell proliferation. In some embodiments, a chemotherapeutic agent and/or oncolytic therapeutic agent may be or comprise platinum compounds (e.g., cisplatin, carboplatin, and oxaliplatin), alkylating agents (e.g., cyclophosphamide, ifosfamide, chlorambucil, nitrogen mustard, thiotepa, melphalan, busulfan, procarbazine, streptozocin, temozolomide, dacarbazine, and bendamustine), antitumor antibiotics (e.g., daunorubicin, doxorubicin, idarubicin, epirubicin, mitoxantrone, bleomycin, mytomycin C, plicamycin, and dactinomycin), taxanes (e.g., paclitaxel and docetaxel), antimetabolites (e.g., 5-fluorouracil, cytarabine, premetrexed, thioguanine, floxuridine, capecitabine, and methotrexate), nucleoside analogues (e.g., fludarabine, clofarabine, cladribine, pentostatin, and nelarabine), topoisomerase inhibitors (e.g., topotecan and irinotecan), hypomethylating agents (e.g., azacitidine and decitabine), proteosome inhibitors (e.g., bortezomib), epipodophyllotoxins (e.g., etoposide and teniposide), DNA synthesis inhibitors (e.g., hydroxyurea), vinca alkaloids (e.g., vicristine, vindesine, vinorelbine, and vinblastine), tyrosine kinase inhibitors (e.g., imatinib, dasatinib, nilotinib, sorafenib, and sunitinib), nitrosoureas (e.g., carmustine, fotemustine, and lomustine), hexamethylmelamine, mitotane, angiogenesis inhibitors (e.g., thalidomide and lenalidomide), steroids (e.g., prednisone, dexamethasone, and prednisolone), hormonal agents (e.g., tamoxifen, raloxifene, leuprolide, bicaluatmide, granisetron, and flutamide), aromatase inhibitors (e.g., letrozole and anastrozole), arsenic trioxide, tretinoin, nonselective cyclooxygenase inhibitors (e.g., nonsteroidal anti-inflammatory agents, salicylates, aspirin, piroxicam, ibuprofen, indomethacin, naprosyn, diclofenac, tolmetin, ketoprofen, nabumetone, and oxaprozin), selective cyclooxygenase-2 (COX-2) inhibitors, or any combination thereof.

In some embodiments, chemotherapeutic agents and/or oncolytic therapeutic agents for anti-cancer treatment comprise biological agents such as tumor-infiltrating lymphocytes, CAR T-cells, antibodies, antigens, therapeutic vaccines (e.g., made from a patient's own tumor cells or other substances such as antigens that are produced by certain tumors), immune-modulating agents (e.g., cytokines, e.g., immunomodulatory drugs or biological response modifiers), checkpoint inhibitors or other immunologic agents. In some embodiments, immunologic agents include immunoglobins, immunostimulants (e.g., bacterial vaccines, colony stimulating factors, interferons, interleukins, therapeutic vaccines, vaccine combinations, viral vaccines) and/or immunosuppressive agents (e.g., calcineurin inhibitors, interleukin inhibitors, TNF alpha inhibitors). In some embodiments, hormonal agents include agents for anti-androgen therapy (e.g., Ketoconazole, ABiraterone, TAK-700, TOK-001, Bicalutamide, Nilutamide, Flutamide, Enzalutamide, ARN-509).

Additional chemotherapeutic agents and/or oncolytic therapeutic agents include immune checkpoint therapeutics (e.g., pembrolizumab, nivolumab, ipilimumab, atezolizumab, avelumab, durvalumab, tremelimumab, or cemiplimab), other monoclonal antibodies (e.g., rituximab, cetuximab, panetumumab, tositumomab, trastuzumab, alemtuzumab, gemtuzumab ozogamicin, bevacizumab, catumaxomab, denosumab, obinutuzumab, ofatumumab, ramucirumab, pertuzumab, nimotuzumab, lambrolizumab, pidilizumab, siltuximab, BMS-936559, RG7446/MPDL3280A, MEDI4736), antibody-drug conjugates (e.g., brentuximab vedotin (ADCETRIS®, Seattle Genetics); ado-trastuzumab emtansine (KADCYLA®, Roche); Gemtuzumab ozogamicin (Wyeth); CMC-544; SAR3419; CDX-011; PSMA-ADC; BT-062; and IMGN901 (see, e.g., Sassoon et al., Methods Mol. Biol. (2013) 1045:1-27; Bouchard et al., Bioorganic Med. Chem. Lett. (2014) 24: 5357-5363), or any combination thereof.

In some embodiments, combined administration of an anti-EMR2 antibody or portion thereof (e.g., one or more CDRs described herein), and/or one or more fusion proteins described herein, or a cell comprising any of the foregoing, and an additional therapeutic agent results in an improvement in cancer treatment to an extent that is greater than one produced by either the anti-EMR2 antibody or the additional therapeutic agent alone (e.g., an improvement in one or more symptom, effect, or measure of the severity of the cancer). The difference between the combined effect and the effect of each agent alone can be a statistically significant difference. In some embodiments, the combined effect can be a synergistic effect. In some embodiments, combined administration of an anti-EMR2 antibody or portion thereof (e.g., one or more CDRs described herein) and/or one or more fusion proteins described herein, or a cell comprising any of the foregoing, and an additional therapeutic agent allows administration of the additional therapeutic agent at a reduced dose, at a reduced number of doses, and/or at a reduced frequency of dosage compared to a standard dosing regimen, e.g., an approved dosing regimen for the additional therapeutic agent.

In some embodiments, treatment methods described herein are performed on subjects for whom other treatments of the medical condition have failed or have had less success in treatment through other means. Additionally, the treatment methods described herein can be performed in conjunction with one or more additional treatments of the medical condition. For instance, the method can comprise administering a cancer treatment regimen, e.g., non-myeloablative chemotherapy, surgery, hormone therapy, and/or radiation, prior to, substantially simultaneously with, or after the administration of an anti-EMR2 antibody described herein or portion thereof (e.g., one or more CDRs described herein) and/or one or more fusion proteins described herein, or composition thereof.

Aspects of the present disclosure involve administration of hematopoietic cells that are genetically modified to have reduced or eliminated expression of EMR2, e.g., in the context of treating a subject in need of such hematopoietic stem cells, which may include, for example, a subject having a hematologic malignancy, such as, e.g., AML, or a premalignancy, such as, e.g., MDS, and undergoing an immunotherapy regimen targeting EMR2, e.g., an EMR2-antibody-drug conjugate or an EMR2 CAR-T or CAR-NK therapy. Such treatment regimen can involve, for example, the following steps: (1) administering a therapeutically effective amount of any of the anti-EMR2 antibodies, EMR2 binding fragments thereof, including fusion proteins, such as e.g., CARs, and cells expressing any of the foregoing, e.g., CAR-T or CAR-NK cells, as described herein or otherwise apparent to the skilled artisan based on the present disclosure; and (2) administering (e.g., infusing or reinfusing) to the patient hematopoietic stem cells, either autologous or allogeneic, where the hematopoietic cells have reduced expression or eliminated expression of EMR2. In some embodiments, the hematopoietic cells are genetically modified to have reduced expression of EMR2. In some embodiments, the hematopoietic cells are genetically modified to have eliminated expression of EMR2. In some embodiments, the hematopoietic cells are genetically modified to have reduced or eliminated expression of an EMR2 epitope bound by an antibody, an EMR2-binding fragment thereof, or portion thereof (e.g., one or more CDRs described herein), and/or one or more fusion proteins described herein.

### CAR and CAR-T Cell Characterization Assays

In some embodiments, one or more CAR characterization assays are used to assess the activity of the CARs and activation of a cell (e.g., T cells) expressing the CARs comprising any of the anti-EMR2 antibodies described herein.

Some suitable CAR characterization assays are provided herein. Exemplary CAR characterization assays include but are not limited to cytotoxicity assays (e.g., chromium (⁵¹Cr)-release assay, luciferase-mediated bioluminescence imaging (BLI) assay, impedance-based assay, and flow cytometry assay), cytokine secretion assays to quantify various cytokines released by CAR-T cells, for example using flow cytometry-based methods, enzyme-linked immunosorbent assays, or reporter constructs (e.g., IL-2 Reporter Systems (IRS)). Additional suitable CAR characterization assays will be apparent to those of skill in the art based on the present disclosure. Exemplary CAR characterization assays are described in, for example, MCB: CAR T Cells: Development, Characterization and Applications. United Kingdom: Elsevier Science, 2022; Cell Reprogramming for Immunotherapy, Springer Nature 2020, Vol. 2097; Zaritskaya et al., Expert Rev Vaccines. (2010) 9(6): 601-616; Xi et al., J Vis Exp. (2019) 153; Mukherjee et al., Mol Ther (2017) 25(8): 1757-1768, which are incorporated by reference herein in their entirety.

In some embodiments, an IL-2 reporter system comprising a minimal nuclear factor of activated T cells (NFAT)-responsive promoter (i.e., an NFAT-responsive reporter system) is used to assess the activity of CARs and activation of a cell (e.g., T cells) expressing the CARs comprising any of the anti-EMR2 antibodies described herein. CAR activation sets in motion an intracellular pathway leading to T-cell activation and effector function of the T cell, which involves NFAT signaling and gene expression (see, e.g., Hogan, Cell Calcium. (2017) 63:66-9). As used herein, the term “NFAT-responsive promoter” refers to a promoter region that is activated by NFAT signaling and promotes expression of a gene that is operably linked to the NFAT-responsive promoter upon activation. In some embodiments, the gene that is operably linked (under control of) the NFAT-responsive promoter encodes a reporter molecule. Various NFAT-responsive reporter systems are described, for example, in PCT Publication No. WO 2023/010118, which is incorporated by reference in its entirety.

### Hematopoietic Cells Deficient in a Lineage-Specific Cell-Surface Antigen

Additionally, the present disclosure provides methods for use of any of the anti-EMR2 antibodies, EMR2 binding fragments thereof, including fusion proteins, or cells comprising any of the foregoing, in combination with inhibition of EMR2 lineage specific antigen. As described herein, such treatment regimens can involve the following steps: (1) administering a therapeutically effective amount of an immune cell described herein (e.g., a T cell) to the patient, where the immune cell comprises a nucleic acid sequence encoding a chimeric antigen receptor (CAR) targeting EMR2 lineage specific antigens; and (2) infusing or reinfusing the patient with hematopoietic stem cells, either autologous or allogeneic, where the hematopoietic cells have reduced or eliminated expression of EMR2 lineage specific antigens.

In some embodiments, the hematopoietic cells are hematopoietic stem cells. Hematopoietic stem cells (HSCs) are capable of giving rise to both myeloid and lymphoid progenitor cells that further give rise to myeloid cells (e.g., monocytes, macrophages, neutrophils, basophils, dendritic cells, erythrocytes, platelets, etc.) and lymphoid cells (e.g., T cells, B cells, NK cells), respectively. HSCs are characterized by the expression of the cell surface marker CD34 (e.g., CD34+), which can be used for the identification and/or isolation of HSCs, and absence of cell surface markers associated with commitment to a cell lineage.

In some embodiments, HSCs are obtained from a subject, such as a mammalian subject. In some embodiments, the mammalian subject is a non-human primate, a rodent (e.g., mouse or rat), a bovine, a porcine, an equine, or a domestic animal. In some embodiments, HSCs are obtained from a human patient, such as a human patient having a hematopoietic malignancy. In some embodiments, HSCs are obtained from a healthy donor (i.e., allogeneic). In some embodiments, HSCs are obtained from the subject to whom the immune cells expressing the chimeric receptors will be subsequently administered (i.e., autologous).

HSCs may be obtained from any suitable source using conventional means known in the art. In some embodiments, HSCs are obtained from a sample from a subject, such as bone marrow sample or from a blood sample. Alternatively, or in addition, HSCs may be obtained from an umbilical cord. In some embodiments, the HSCs are from bone marrow or peripheral blood mononuclear cells (PBMCs). In general, bone marrow cells may be obtained from iliac crest, femora, tibiae, spine, rib or other medullary spaces of a subject. Bone marrow may be taken out of the patient and isolated through various separations and washing procedures known in the art. An exemplary procedure for isolation of bone marrow cells comprises the following steps: a) extraction of a bone marrow sample; b) centrifugal separation of bone marrow suspension in three fractions and collecting the intermediate fraction, or buffycoat; c) the buffycoat fraction from step (b) is centrifuged one more time in a separation fluid, commonly Ficoll™, and an intermediate fraction which contains the bone marrow cells is collected; and d) washing of the collected fraction from step (c) for recovery of re-transfusable bone marrow cells.

HSCs typically reside in the bone marrow but can be mobilized into the circulating blood by administering a mobilizing agent in order to harvest HSCs from the peripheral blood. In some embodiments, the subject from which the HSCs are obtained is administered a mobilizing agent, such as granulocyte colony-stimulating factor (G-CSF). The number of the HSCs collected following mobilization using a mobilizing agent is typically greater than the number of cells obtained without use of a mobilizing agent.

In some embodiments, a sample is obtained from a subject and is then enriched for a desired cell type (e.g., CD34+/EMR2-cells). For example, PBMCs and/or CD34+ hematopoietic cells can be isolated from blood as described herein. Cells can also be isolated from other cells, for example by isolation and/or activation with an antibody binding to an epitope on the cell surface of the desired cell type. Another method that can be used includes negative selection using antibodies to cell surface markers to selectively enrich for a specific cell type without activating the cell by receptor engagement.

Populations of HSC can be expanded prior to or after genetically engineering the HSC to become deficient in a lineage specific cell-surface antigen. The cells may be cultured under conditions that comprise an expansion medium comprising one or more cytokines, such as stem cell factor (SCF), Flt-3 ligand (FLt3L), thrombopoietin (TPO), Interleukin 3 (IL-3), or Interleukin 6 (IL-6). The cell may be expanded for about 1, 2, 3, 4, 5, 6, 7, 8, 9, 10, 11, 12, 13, 14, 15, 16, 17, 18, 19, 20, 21, 22, 23, 23, 25 days or any range necessary. In some embodiments, HSCs are expanded after isolation of a desired cell population (e.g., CD34+/EMR2-) from a sample obtained from a subject and prior to genetic engineering. In some embodiments, the HSC are expanded after genetic engineering, thereby selectively expanding cells that have undergone the genetic modification and are deficient in a lineage-specific cell-surface antigen. In some embodiments, a cell (“a clone”) or several cells having a desired characteristic (e.g., phenotype or genotype) following genetic modification may be selected and independently expanded.

In some embodiments, the hematopoietic cells are genetically engineered to be deficient in a cell-surface lineage-specific antigen. In some embodiments, the hematopoietic cells are genetically engineered to be deficient in the same cell-surface lineage-specific antigen that is targeted by the agent. As used herein, a hematopoietic cell is considered to be deficient in a cell-surface lineage-specific antigen if hematopoietic cell has substantially reduced expression of the cell-surface lineage-specific antigen as compared to a naturally occurring hematopoietic cell of the same type as the genetically engineered hematopoietic cell (e.g., is characterized by the presence of the same cell surface markers, such as CD34).

In some embodiments, the hematopoietic cell has no detectable expression of the cell-surface lineage-specific antigen. The expression level of a cell-surface lineage-specific antigen can be assessed by any means known in the art. For example, the expression level of a cell surface lineage-specific antigen can be assessed by detecting the antigen with an antigen specific antibody (e.g., flow cytometry methods, Western blotting).

In some embodiments, the expression of the cell-surface lineage-specific antigen on the genetically engineered hematopoietic cell is compared to the expression of the cell-surface lineage-specific antigen on a naturally occurring hematopoietic cell. In some embodiments, the genetic engineering results in a reduction in the expression level of the cell-surface lineage-specific antigen by at least about 50%, 60%, 70%, 80%, 90%, 91%, 92%, 93%, 94%, 95%, 96%, 97%, 98%, or 99% as compared to the expression of the cell-surface lineage-specific antigen on a naturally occurring hematopoietic cell. In some embodiments, the hematopoietic cell is deficient in the whole endogenous gene encoding the cell-surface lineage-specific antigen. In some embodiments, the whole endogenous gene encoding the cell-surface lineage-specific antigen has been deleted. In some embodiments, the hematopoietic cell comprises a portion of endogenous gene encoding the cell-surface lineage-specific antigen. In some embodiments, the hematopoietic cell expressing a portion (e.g., a truncated protein) of the cell-surface lineage specific antigen. In other embodiments, a portion of the endogenous gene encoding the cell surface lineage-specific antigen has been deleted. In some embodiments, at least 10%, 20%, 30%, 40%, 50%, 60%, 70% or more of the gene encoding the cell-surface lineage-specific antigen has been deleted.

As will be appreciated by one of ordinary skill in the art, a portion of the nucleotide sequence encoding the cell-surface lineage-specific antigen may be deleted or one or more non-coding sequences, such that the hematopoietic cell is deficient in the antigen (e.g., has substantially reduced expression of the antigen).

In some embodiments, the cell-surface lineage-specific antigen is EMR2. In some embodiments, a portion of EMR2 is deleted. In some embodiments, an epitope of EMR2 to which an anti-EMR2 antibody, or antigen binding fragment thereof, binds/recognizes is deleted. In some embodiments, the cell is a hematopoietic cell (e.g., a hematopoietic stem or progenitor cell) having reduced EMR2 expression (e.g., reduced 1-10%, 10-20%, 20-30%, 30-40%, 40-50%, 50-60%, 60-70%, 70-80%, 80-90%, 90-95%, or more than 95%) relative to a wild-type counterpart cell. In some embodiments, a cell having reduced or eliminated expression of EMR2 has expresses a variant of EMR2. In some embodiments, the variant of EMR2 comprises an amino acid substitution, an amino acid deletion, or an amino acid insertion at one or more corresponding amino acid positions in EMR2 (e.g., a variant of EMR2 lacking an epitope, such as an extracellular epitope targeted by an antibody or antigen binding fragment thereof described herein).

Any of the genetically engineering hematopoietic cells, such as HSCs, that are deficient in a cell-surface lineage-specific antigen can be prepared by routine methods or by methods described herein. In some embodiments, the genetic engineering is performed using genome editing. As used herein, “genome editing” refers to a method of modifying the genome, including any protein-coding or non-coding nucleotide sequence, of an organism to knock out the expression of a target gene. In general, genome editing methods involve use of an endonuclease that is capable of cleaving the nucleic acid of the genome, for example at a targeted nucleotide sequence. Repair of the double-stranded breaks in the genome may be repaired introducing mutations and/or exogenous nucleic acid may be inserted into the targeted site.

Genome editing methods are generally classified based on the type of endonuclease that is involved in generating double stranded breaks in the target nucleic acid. These methods include use of zinc finger nucleases (ZFN), transcription activator-like effector-based nuclease (TALEN), meganucleases, and CRISPR/Cas systems. Methods of editing the genome of HSCs described herein can be found, e.g., in PCT Publication Nos. WO 2017/066760, WO 2020/047164, WO 2021/041971, and WO 2022/047168, all of which are incorporated by reference in their entirety. Examples of CRISPR/Cas systems including Cas endonucleases and gRNAs sequences for editing the EMR2 gene can be found, for example, in PCT Publication Nos. WO 2023/086422 and WO 2023/043858, which are incorporated by reference in their entireties. In some embodiments, a cell having reduced or eliminated expression of EMR2 (e.g., a cell having reduced or eliminated expression of wild-type EMR2 and/or expressing a variant of EMR2 including, but not limited to, a hematopoietic cell, such as a hematopoietic stem or progenitor cell or an immune cell described herein) is engineered using a CRISPR/Cas system comprising a Cas endonuclease (e.g., Cas nuclease and variants thereof, such as base editors including, but not limited to, adenosine base editors (ABEs)) and a gRNA described in PCT Publication No. WO 2023/086422 or WO 2023/043858. In some embodiments, a gRNA used for editing an EMR2 gene comprises a spacer sequence of CUUGGCCAAUAACACCAUCC (SEQ ID NO: 104) or GUGGUACCUGCUGGCUGAGG (SEQ ID NO: 105).

As described herein, agents comprising an antigen-binding domain that binds to a cell-surface lineage-specific antigen (e.g., EMR2 antibodies, fusion proteins, including EMR2 CAR) may be administered to a subject in combination with hematopoietic cells that are deficient for the cell-surface lineage-specific antigen (i.e., EMR2).

In some embodiments, the agents and/or the hematopoietic cells may be mixed with a pharmaceutically acceptable carrier to form a pharmaceutical composition, which is also within the scope of the present disclosure.

To perform the methods described herein, an effective amount of the agent comprising an antigen-binding domain that binds to a cell-surface lineage-specific antigen and an effective amount of hematopoietic cells can be co-administered to a subject in need of the treatment.

As described herein, the hematopoietic cells and/or immune cells expressing chimeric receptors may be autologous to the subject. i.e., the cells are obtained from the subject in need of the treatment, genetically engineered to be deficient for expression of the cell-surface lineage-specific antigen or for expression of the chimeric receptor constructs, and then administered to the same subject. Administration of autologous cells to a subject may result in reduced rejection of the host cells as compared to administration of non-autologous cells. Alternatively, the host cells are allogeneic cells, i.e., the cells are obtained from a first subject, genetically engineered to be deficient for expression of the cell-surface lineage-specific antigen or for expression of the chimeric receptor constructs, and administered to a second subject that is different from the first subject but of the same species. For example, allogeneic immune cells may be derived from a human donor and administered to a human recipient who is different from the donor.

In some embodiments, the immune cells and/or hematopoietic cells are allogeneic cells and have been further genetically engineered to reduced graft-versus-host disease. For example, as described herein, the hematopoietic stem cells may be genetically engineered (e.g., using genome editing) to have reduced expression of CD45RA.

In some embodiments, the immune cells expressing any of the chimeric receptors described herein are administered to a subject in an amount effective in to reduce the number of target cells (e.g., cancer cells) by least 20%, e.g., 50%, 80%, 100%, 2-fold, 5-fold, 10-fold, 20-fold, 50-fold, 100-fold or more.

In some embodiments, a subject is administered an agent that comprises an antigen-binding domain that binds a cell-surface lineage-specific antigen and a population of hematopoietic cells deficient in the cell-surface lineage-specific antigen. Accordingly, in such therapeutic methods, the agent recognizes (binds) a target cell expressing the cell-surface lineage-specific antigen for targeted killing. The hematopoietic cells that are deficient in the antigen allow for repopulation of a cell type that is targeted by the agent. In some embodiments, the treatment of the patient can involve the following steps: (1) administering a therapeutically effective amount of an agent targeting a cell-surface lineage-specific antigen to the patient and (2) infusing or reinfusing the patient with hematopoietic stem cells, either autologous or allogenic, where the hematopoietic cells have reduced expression of a lineage specific disease-associated antigen. In some embodiments, the treatment of the patient can involve the following steps: (1) administering a therapeutically effective amount of an immune cell expressing a chimeric receptor to the patient, wherein the immune cell comprises a nucleic acid sequence encoding a chimeric receptor that binds a cell-surface lineage-specific, disease associated antigen; and (2) infusing or reinfusing the patient with hematopoietic cells (e.g., hematopoietic stem cells), either autologous or allogenic, where the hematopoietic cells have reduced expression of a lineage specific disease-associated antigen.

The efficacy of the therapeutic methods using an agent comprising an antigen-binding fragment that binds a cell-surface lineage-specific antigen and a population of hematopoietic cells deficient in the cell-surface lineage-specific antigen may be assessed by any method known in the art and would be evident to a skilled medical professional. For example, the efficacy of the therapy may be assessed by survival of the subject or cancer burden in the subject or tissue or sample thereof. In some embodiments, the efficacy of the therapy is assessed by quantifying the number of cells belonging to a particular population or lineage of cells. In some embodiments, the efficacy of the therapy is assessed by quantifying the number of cells presenting the cell-surface lineage-specific antigen. In some embodiments, the agent comprising an antigen-binding fragment that binds to the cell-surface lineage-specific antigen and the population of hematopoietic cells is administered concomitantly.

In some embodiments, the agent comprising an antigen-binding fragment that binds a cell-surface lineage-specific antigen (e.g., immune cells expressing a chimeric receptor as described herein) is administered prior to administration of the hematopoietic cells. In some embodiments, the agent comprising an antigen-binding fragment that binds a cell-surface lineage-specific antigen (e.g., immune cells expressing a chimeric receptor as described herein) is administered at least about 1 day, 2 days, 3 days, 4 days, 5 days, 6 days, 1 week, 2 weeks, 3 weeks, 4 weeks, 5 weeks, 6 weeks, 7 weeks, 8 weeks, 9 weeks, 10 weeks, 11 weeks, 12 weeks, 3 months, 4 months, 5 months, 6 months or more prior to administration of the hematopoietic cells.

In some embodiments, the hematopoietic cells are administered prior to the agent comprising an antigen-binding fragment that binds a cell-surface lineage-specific antigen (e.g., immune cells expressing a chimeric receptor as described herein). In some embodiments, the population of hematopoietic cells is administered at least about 1 day, 2 days, 3 days, 4 days, 5 days, 6 days, 1 week, 2 weeks, 3 weeks, 4 weeks, 5 weeks, 6 weeks, 7 weeks, 8 weeks, 9 weeks, 10 weeks, 11 weeks, 12 weeks, 3 months, 4 months, 5 months, 6 months or more prior to administration of the agent comprising an antigen-binding fragment that binds to the cell-surface lineage-specific antigen.

In some embodiments, the agent targeting the cell-surface lineage-specific antigen and the population of hematopoietic cells are administered at substantially the same time. In some embodiments, the agent targeting the cell-surface lineage-specific antigen is administered and the patient is assessed for a period of time, the population of hematopoietic cells is administered, and the patient is assessed for a period of time, after which agent targeting the cell-surface lineage-specific antigen is administered.

Also within the scope of the present disclosure are multiple administrations (e.g., doses) of the agents and/or populations of hematopoietic cells. In some embodiments, the agents and/or populations of hematopoietic cells are administered to the subject once. In some embodiments, agents and/or populations of hematopoietic cells are administered to the subject more than once (e.g., at least 2, 3, 4, 5, or more times). In some embodiments, the agents and/or populations of hematopoietic cells are administered to the subject at a regular interval, e.g., every six months.

In some embodiments, the subject is a human subject having a hematopoietic malignancy. As used herein a hematopoietic malignancy refers to a malignant abnormality involving hematopoietic cells (e.g., blood cells, including progenitor and stem cells). Examples of hematopoietic malignancies include, without limitation, Hodgkin's lymphoma, non-Hodgkin's lymphoma, leukemia, or multiple myeloma. Leukemias include acute myeloid leukemia, chronic myelogenous leukemia, chronic lymphoblastic leukemia, and chronic lymphoid leukemia.

### General Techniques

The practice of the present disclosure will employ, unless otherwise indicated, conventional techniques of molecular biology (including recombinant techniques), microbiology, cell biology, biochemistry, and immunology, which are within the skill of the art. Such techniques are explained fully in the literature, such as Molecular Cloning: A Laboratory Manual, second edition (Sambrook, et al., 1989) Cold Spring Harbor Press; Oligonucleotide Synthesis (M. J. Gait, ed. 1984); Methods in Molecular Biology, Humana Press; Cell Biology: A Laboratory Notebook (J. E. Cellis, ed., 1989) Academic Press; Animal Cell Culture (R. I. Freshney, ed. 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds. 1993-8) J. Wiley and Sons; Methods in Enzymology (Academic Press, Inc.): Handbook of Experimental Immunology (D. M. Weir and C. C. Blackwell, eds.): Gene Transfer Vectors for Mammalian Cells (J. M. Miller and M. P. Calos, eds., 1987; Current Protocols in Molecular Biology (F. M. Ausubel, et al. eds. 1987); PCR: The Polymerase Chain Reaction, (Mullis, et al., eds. 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practice approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds. Harwood Academic Publishers, 1995); DNA Cloning: A practical Approach, Volumes I and II (D. N. Glover ed. 1985); Nucleic Acid Hybridization (B. D. Hames & S. J. Higgins eds. (1985; Transcription and Translation (B. D. Hames & S. J. Higgins, eds. (1984; Animal Cell Culture (R. I. Freshney, ed. (1986; Immobilized Cells and Enzymes (IRL Press, (1986; and B. Perbal, A practical Guide To Molecular Cloning (1984); F. M. Ausubel et al. (eds.).

Without further elaboration, it is believed that one skilled in the art can, based on the above description, utilize the present disclosure to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

All publications, patent applications, patents, and other references mentioned herein are incorporated by reference in their entirety. In addition, the materials, methods, and examples are illustrative only and not intended to be limiting. Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described herein.

The disclosure is further illustrated by the following examples. The examples are provided for illustrative purposes only. They are not to be construed as limiting the scope or content of the disclosure in any way.

### EXAMPLES

### Example 1. Generation of Novel Antibodies Against EMR2

A fully human scFv phage display library and a human VH phage display library were panned and screened to identify single domain antibodies that specifically recognize EMR2. A recombinant Navi-His-tag EMR2 protein was used during panning. Human myeloid MOLM13 cells were used as an EMR2-positive cell line, and HEK-293 cells were used as EMR2 negative cells. A purified anti-human EMR2 antibody was used as a positive control. Specific binding activity of identified binders was confirmed by FACS flow cytometry and ELISA as described below. The scFv and heavy chain variable region sequences of the identified EMR2 binders are included herein (Tables 1-11).

### Flow Cytometry Analysis of EMR2 Binders

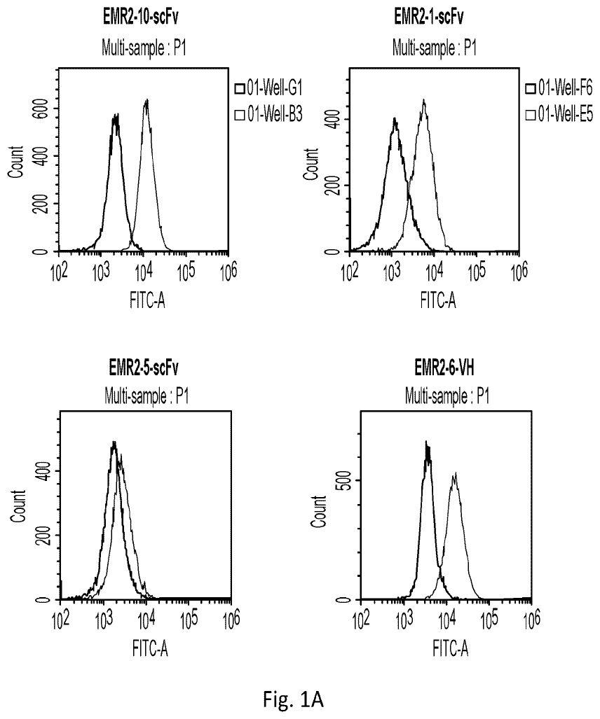

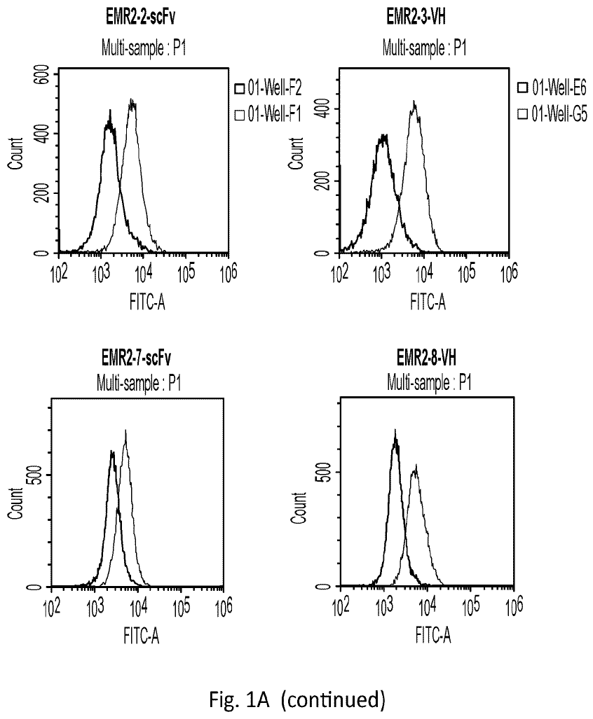

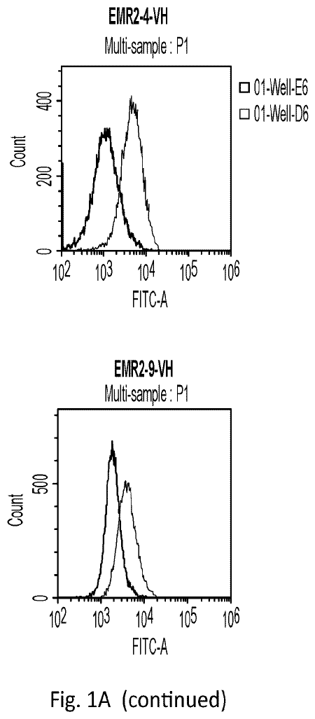

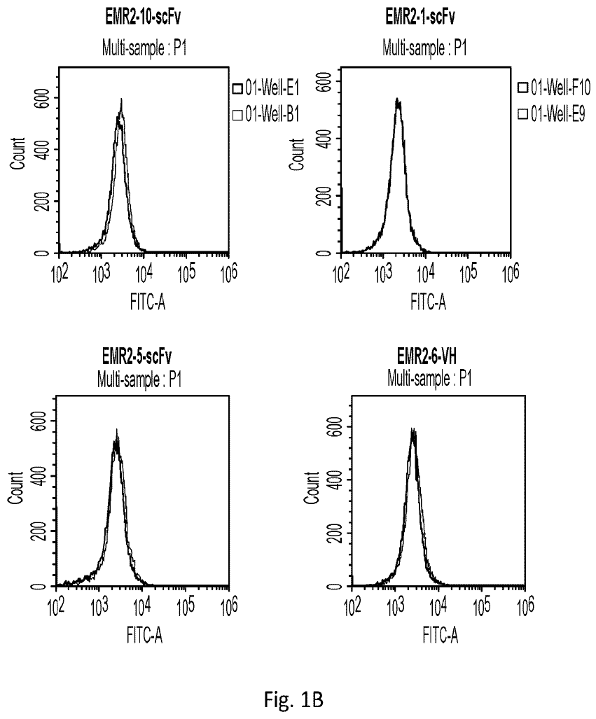

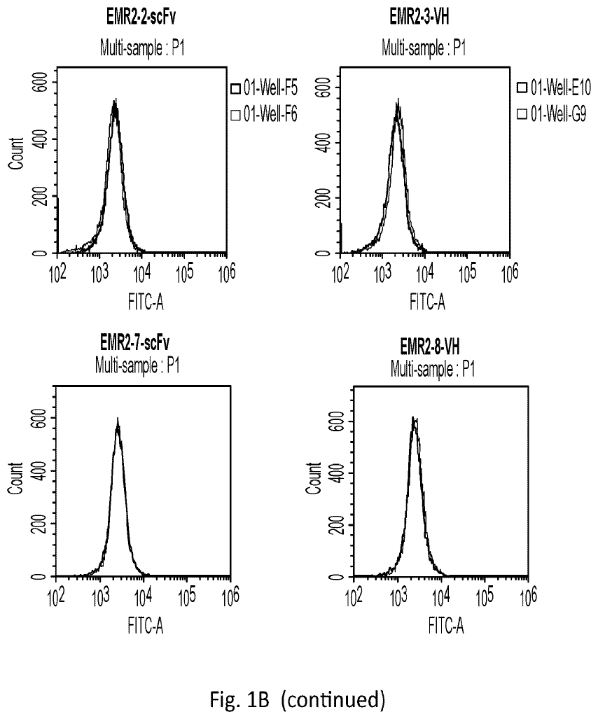

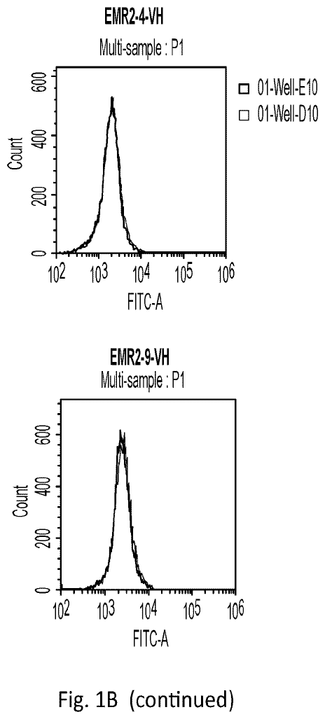

Anti-EMR2 scFv binders (Anti-EMR2 Clone EMR2-10-scFv (Table 10), Anti-EMR2 Clone EMR2-1-scFv (Table 1), Anti-EMR2 Clone EMR2-2-scFv (Table 2), Anti-EMR2 Clone EMR2-5-scFv (Table 5) and Anti-EMR2 Clone EMR2-7-scFv (Table 7)) and VH binders (Anti-EMR2 Clone EMR2-3-VH (Table 3), Anti-EMR2 Clone EMR2-4-VH (Table 4), Anti-EMR2 Clone EMR2-6-VH (Table 6), Anti-EMR2 Clone EMR2-8-VH (Table 8), Anti-EMR2 Clone EMR2-9-VH (Table 9)) were tested for binding ability to EMR2-expressing cells. Binding was assessed by staining of cells with primary and secondary labeled antibodies vs. unstained controls. MOLM13 WT or HEK-293 control cells were counted, washed in PBS and mixed with 1 ml of Fixable Viability dye eFluor 780 (diluted at 1:1000 in PBS) then incubated at room temperature for 10 minutes to distinguish Live/Dead cells. Subsequently, the cells were washed once with FACS Buffer (PBS with 2% FBS) and incubated with human TruStain FcX™ (Fc block diluted at 1:10 in FACS buffer) at room temperature for 5 minutes. Fc blocked cells were stained with 50 μL of the EMR2 Flag-tagged binders (at 200 nM concentrations) and incubated at 4 °C for 30 minutes in the dark. Cells were subsequently stained with 100 μL of a secondary antibody (FITC Rabbit anti-FLAG-tag diluted at 1:1000) and incubated at 4 °C for 30 minutes in the dark. Cells were washed and flow cytometric analysis was performed on the NovoCyte Quanteon flow cytometer (AgilentTechnologies) and data analysis was performed using FlowJo™. See FIGS. 1A and 1B.

As shown in FIGS. 1A and 1B, EMR2 binders displayed significantly higher levels of binding to target cells expressing EMR2, as compared to HEK-293 cells. These data demonstrate specific binding of binder clones to AML cell lines.

### Example 2. Characterization of scFv and VH Binders

### Non-Specific Binding Assessment by ELISA

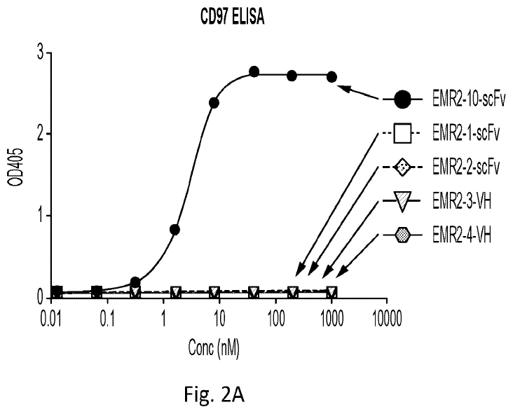

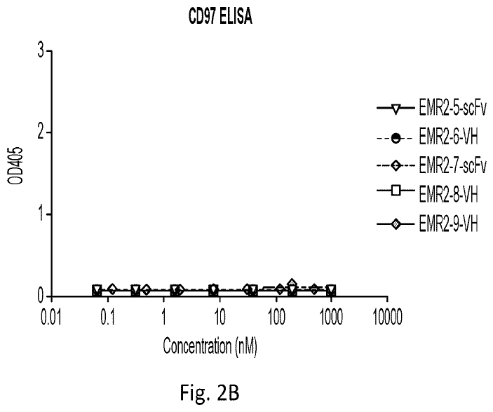

Cross reactivity of the EMR2 binders with CD97 was assessed by Enzyme-Linked Immunosorbent Assay (ELISA). Mean fluorescent intensity values for each binder were obtained at concentrations ranging from 0.01 nM to 1000 nM. Each binder was applied to a CD97-coated microtiter plate. Optical density measurements were obtained at 405 nm and plotted against binder concentrations. As shown in FIGS. 2A and 2B, the majority of the tested EMR2 binders did not cross-react with CD97, with the exception of EMR2-10-scFv, which demonstrated cross reactivity at concentrations above 1 nM.

### Binding Affinity Assessment by ELISA and Flow Cytometry

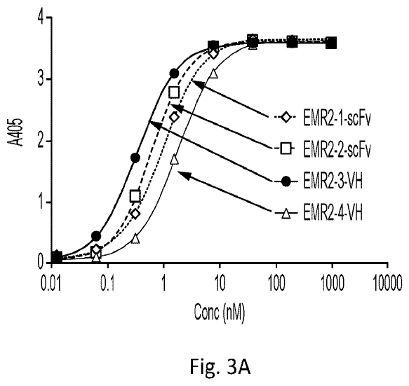

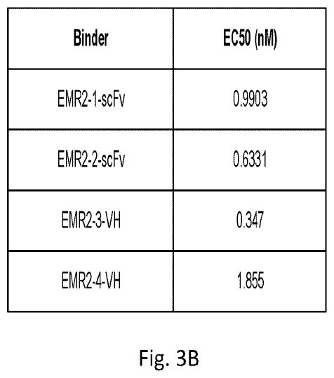

Binding affinity of the anti-EMR2 binders to recombinant EMR2 was measured by ELISA and flow cytometry methods. Mean fluorescent intensity values for each binder were obtained at concentrations ranging from 0.01 nM to 1000 nM. Binders were applied to recombinant human EMR2-coated microtiter plates. Optical density measurements were obtained at 405 nm and plotted against binder concentrations. FIG. 3A shows binding affinities of the indicated binders at tested concentrations. Binders EMR2-1-scFv, EMR2-2-scFv, and EMR2-3-VH demonstrated binding affinities lower than 1 nM (FIG. 3B).

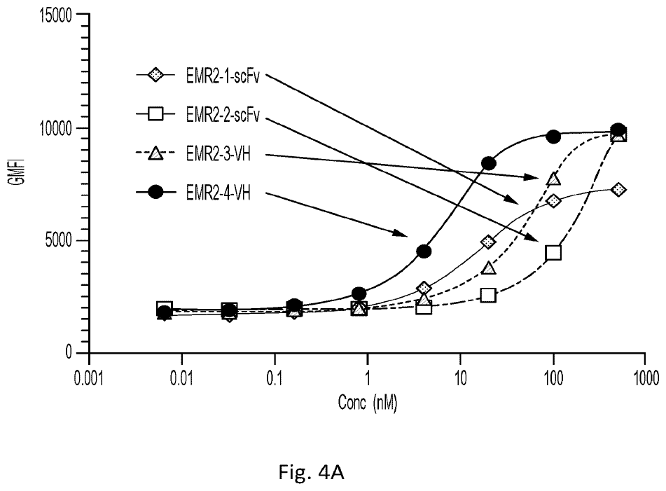

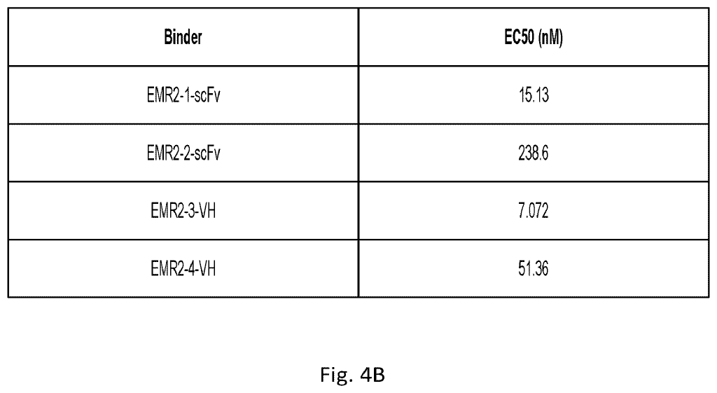

Binding affinity of the EMR2 binders to MOLM13 cells was measured by flow cytometry. Briefly, the EMR2 binders were incubated with a MOLM13 cell line, which expresses EMR2. Cells were washed and flow cytometric analysis was performed on the NovoCyte Quanteon flow cytometer (Agilent Technologies), and data analysis was performed using FlowJo™. Mean fluorescent intensity values for each binder were obtained at concentrations ranging from 0.01 nM to 1000 nM. EMR2-3-VH showed the highest binding affinity (FIG. 4A). FIG. 4B shows a table with the half-maximal binding affinity (EC50) values for each binder.

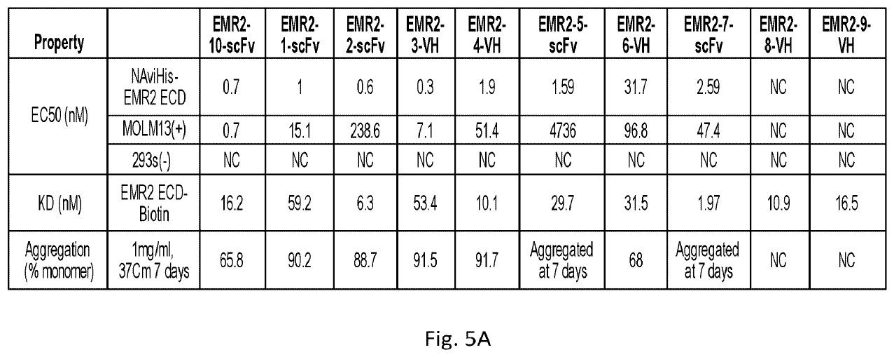

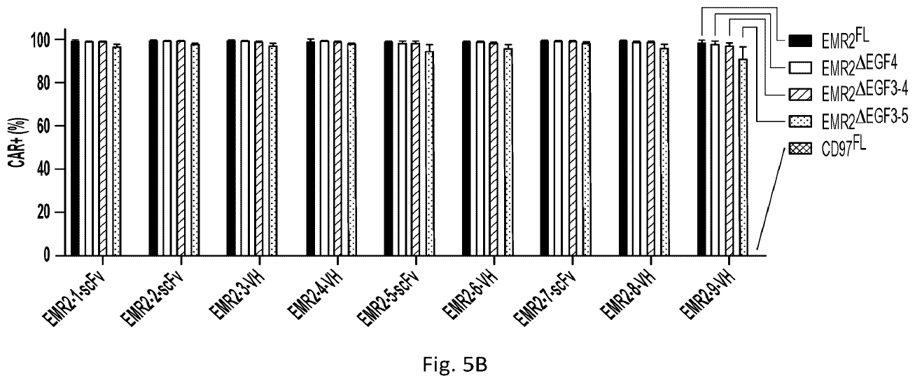

FIG. 5A shows a summary of binder characteristics of binders obtained by panning using EMR2 against scFv and VH libraries. Results of binder characterization are shown for flow cytometry and ELISA based EC50 measurements, CD97 cross-reactivity, stability assessment, and binding affinities. FIG. 5B shows binding to various EMR2 isoforms. Binders were selected for further testing as part of a chimeric antigen receptor (CAR).

### Example 3. EMR2 Binder CAR Constructs

### CAR Constructs

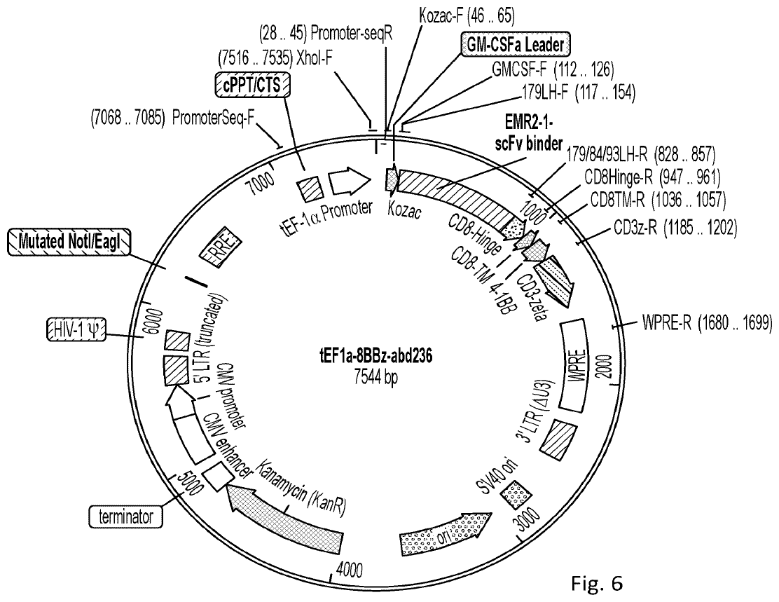

CAR constructs were developed with selected EMR2-specific binders described herein. Exemplary CAR constructs comprise a single chain antibody or single domain antibody linked with a CD8a transmembrane domain, paired with a 4-1BB co-stimulatory domain, and a CD3ξ (zeta) signaling domain. The CAR sequences were cloned in a third-generation lentiviral plasmid. All restriction enzymes were purchased from New England Biolabs (Ipswich, MA, USA). The sequences of the CAR constructs were confirmed by sequencing. Exemplary CAR construct may comprise a nucleotide sequence encoding a EMR2-specific binder provided by any of SEQ ID NOs: 60, 62, 64, 66, 68, 70, 72, 74, 76, 78, 80, 82, 84, 86, 88, 90, 92, 94, 96, 98, 100, and 102 (see, e.g., Tables 1-11). The exemplary plasmid map encoding an EMR2 binder EMR2-lis shown in FIG. 6.

### Flow Cytometry Analysis of EMR2 CAR-T Expression

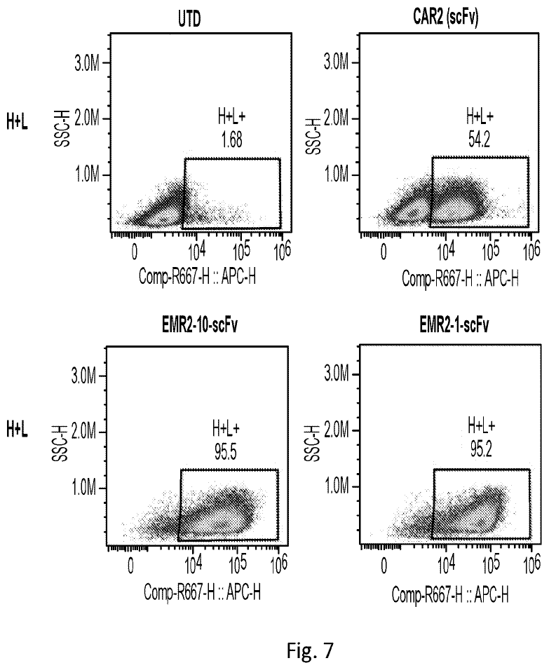

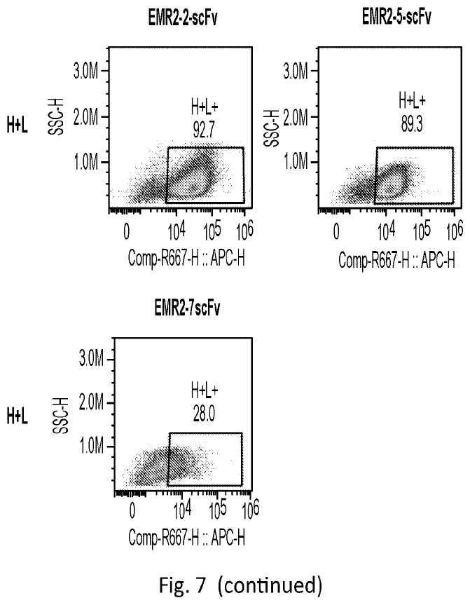

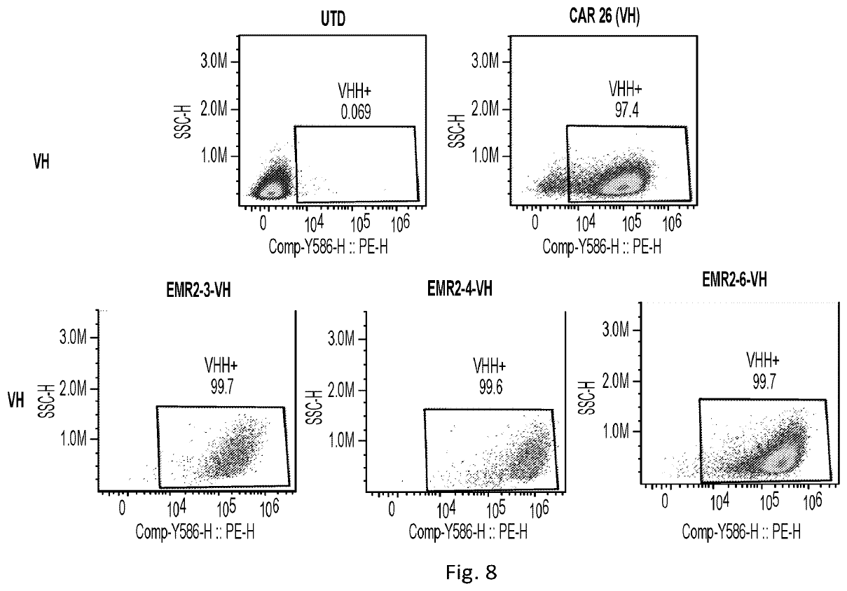

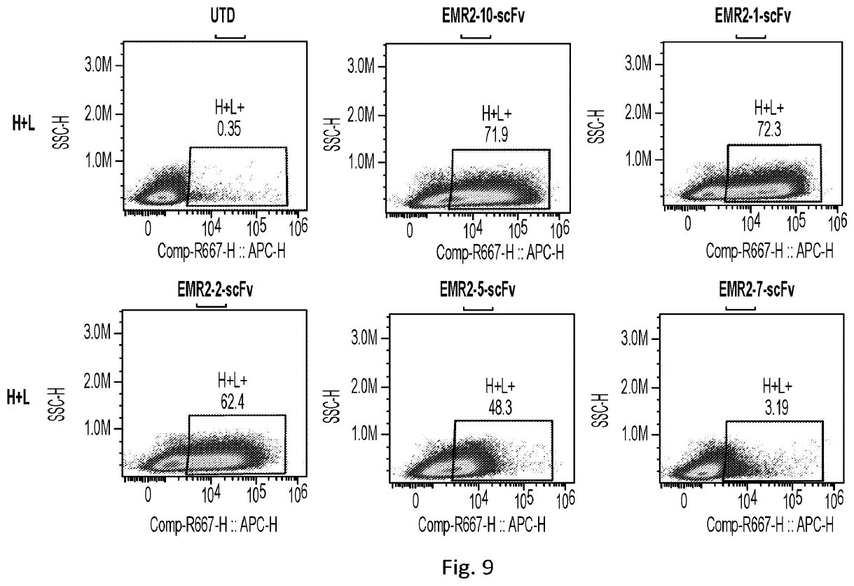

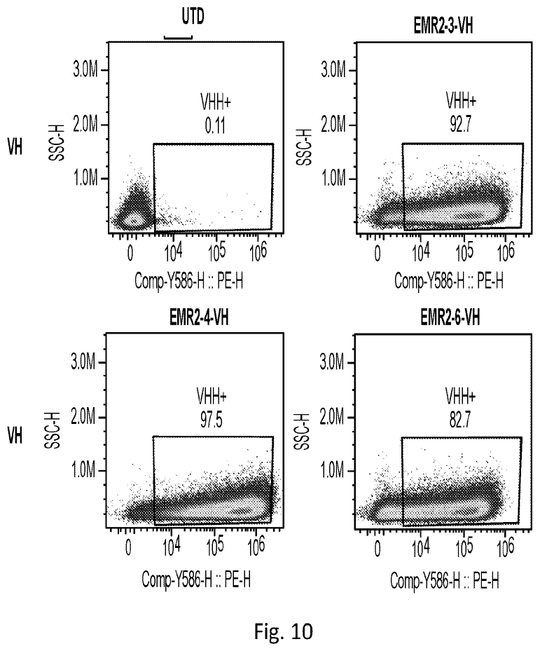

Exemplary CAR constructs were transduced into Jurkat cells and primary T cells. Surface expression of the CARs was detected on day 5 following transduction using an anti-IgG (H+L) or anti-VH antibody. Untransduced cells and CAR2 (scFv) or CAR26 (VH), both CD33-directed CARs were used as positive controls to stain for CAR expression using anti-IgG (H+L) and anti-VH antibodies. FIGS. 7-10 show that all of the CARs containing the scFv binders or VH binders were expressed on the surface of Jurkat cells (FIG. 7 and FIG. 8), as well as in primary T cells (FIG. 9 and FIG. 10).

### Characterization of EMR2-Directed CAR Expressing Cells

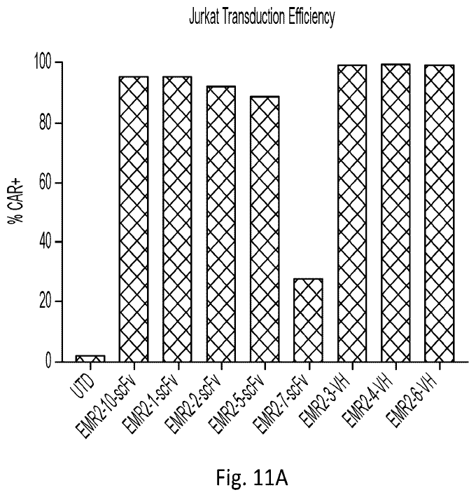

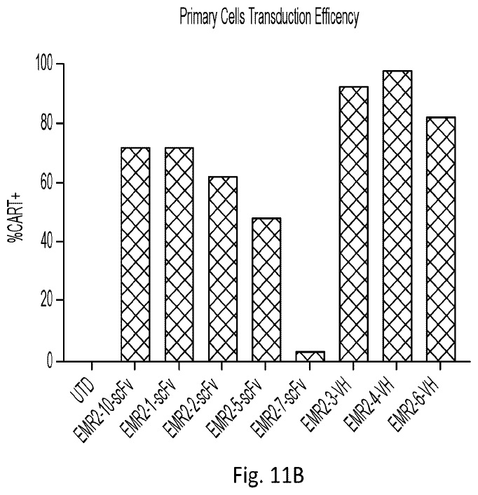

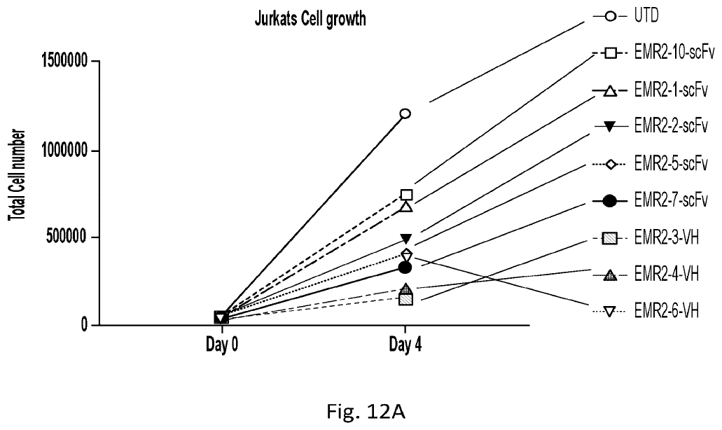

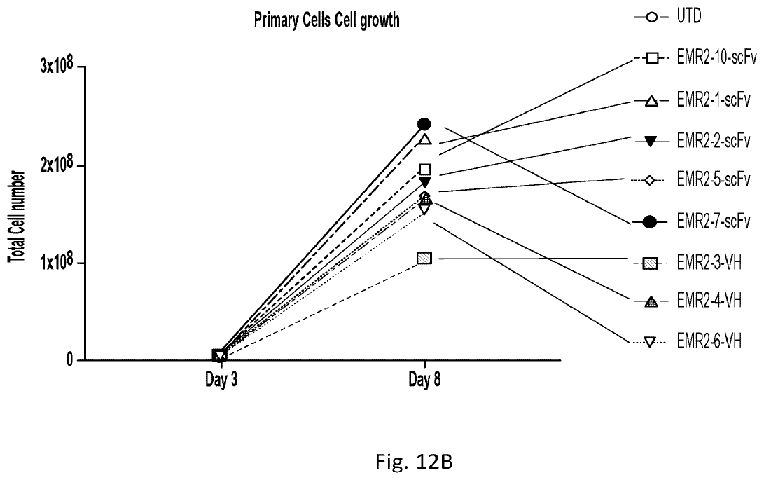

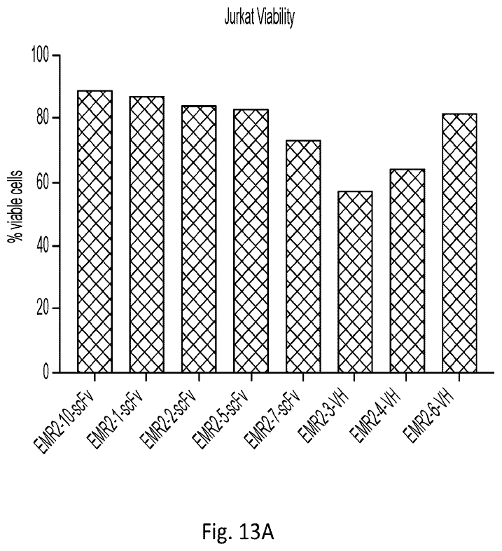

Transduction efficiency, cell growth, and cell viability were analyzed in Jurkat and primary T cells for each of the CAR constructs containing the exemplary EMR2 scFv and VH binders. As shown in FIGS. 11A and 11B, the majority of CAR constructs were efficiency transduced into Jurkat cells and primary T cells. Cell growth was assessed by enumerating the total cell number over time (FIGS. 12A and 12B). Cell viability was measured after transduction with scFv and VH CARs in both Jurkat and primary T cell lines (FIGS. 13A and 13B).

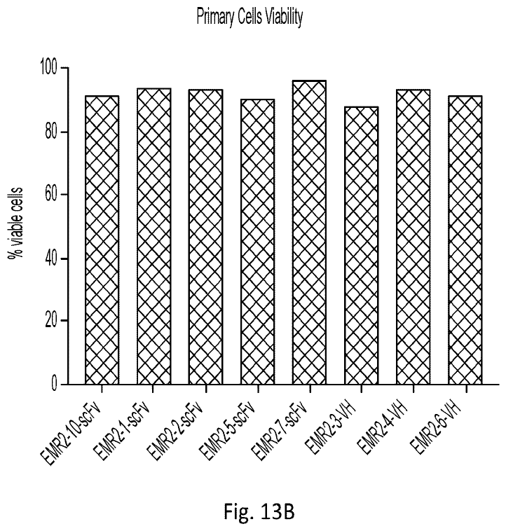

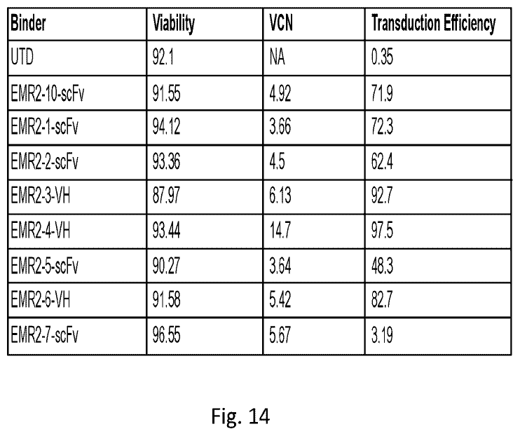

Cell viability, vector copy number (VCN), and transduction efficiency were also assessed in primary T cells transduced with the exemplary CAR constructs and compared to untransduced cells (UTD) (FIG. 14).

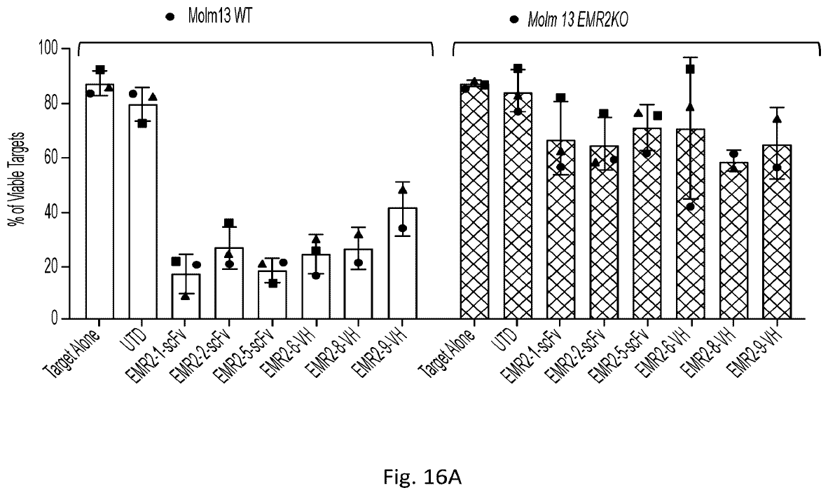

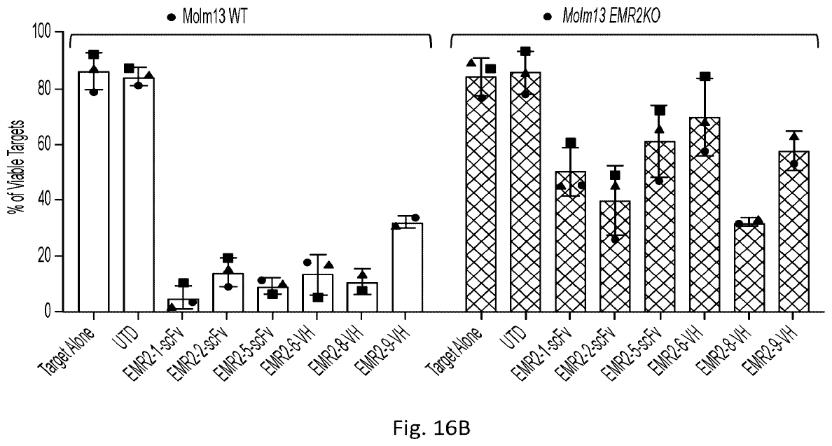

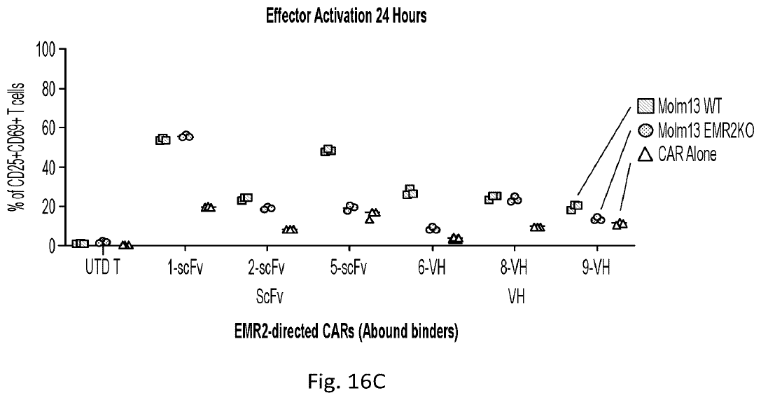

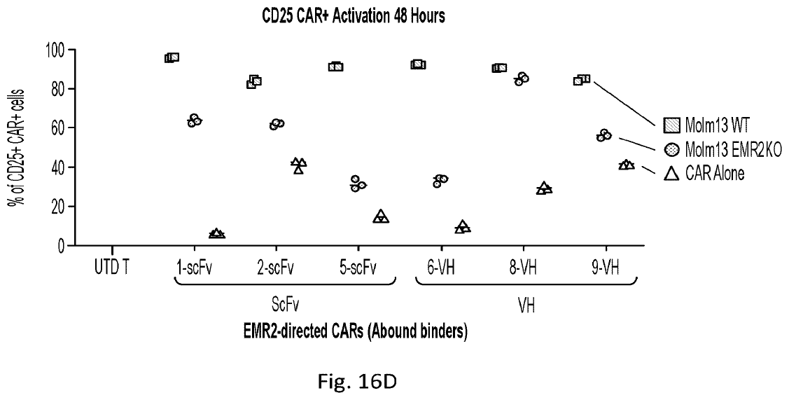

Cytotoxic activity of EMR2-directed CARs was assessed by flow cytometry. MOLM13 WT and MOLM13 EMR2KO were used as target cells and incubated with exemplary EMR2-directed CAR T cells (effector cells). The viability was calculated compared to untransduced controls (UTD) and presented as the change in viability (delta viability). The EMR2-directed CARs exhibited cytotoxic activity toward the target cells as shown in FIGS. 16A and 16B.

The CAR constructs containing the indicated EMR2 scFv and VH binders were tested for activation of the effector cells by flow cytometry. Briefly, effector cells expressing the EMR2-directed CARs were incubated in the presence of target cells (MOLM13 WT or MOLM13 EMR2KO) or alone (CAR alone). Activation was assessed by measuring the percentage of CD25 and CD69 positive T cells in the total effector cell population at 24 hr (FIG. 16C) or in the CAR+ effector population at 48 hr (FIG. 16D). Results demonstrated specific activation of EMR2-directed CAR T cells.

### Analysis of Cytokine Production

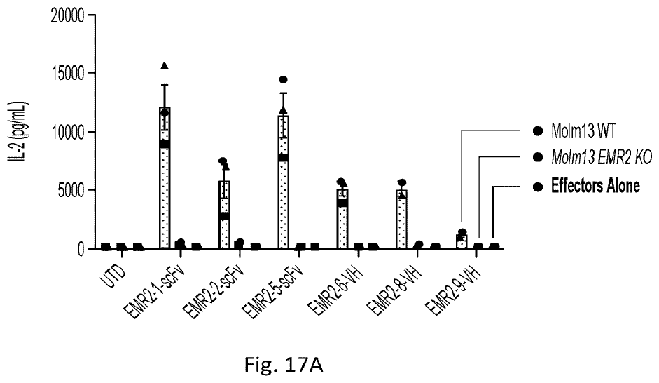

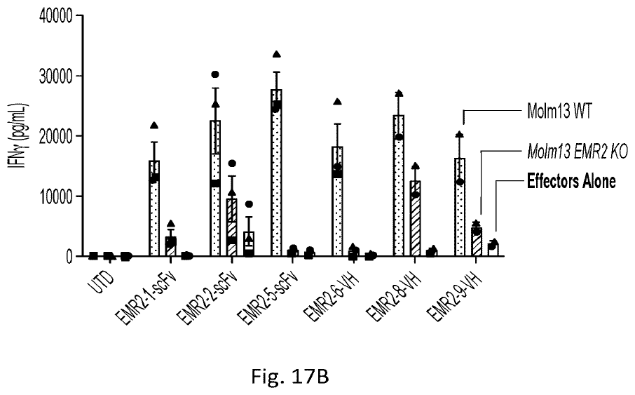

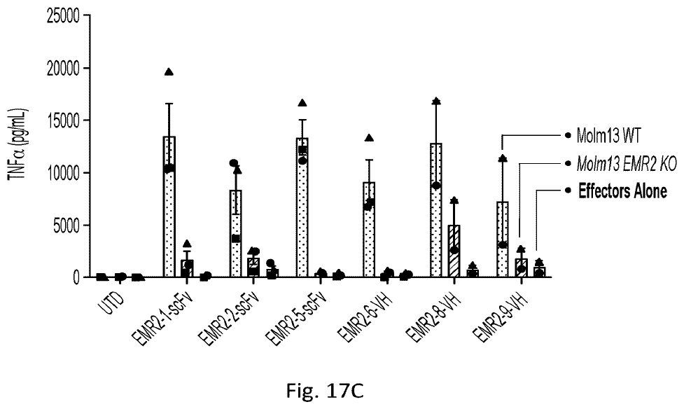

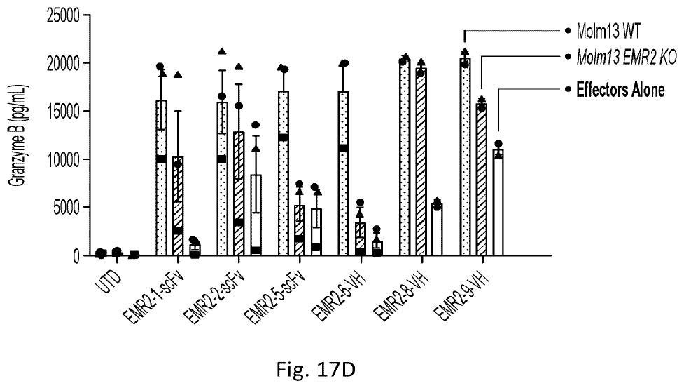

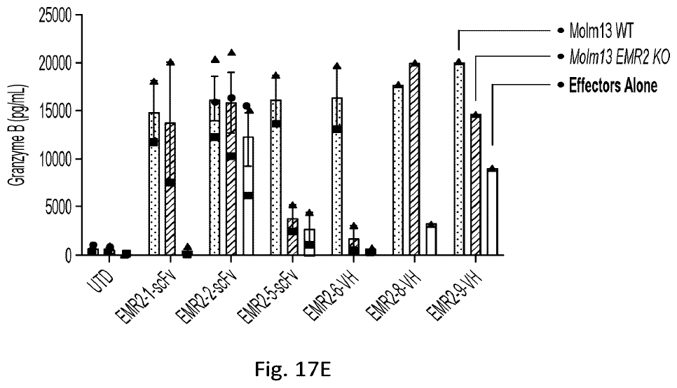

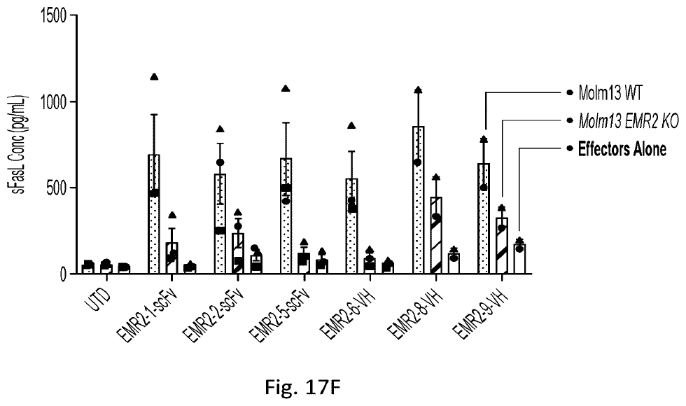

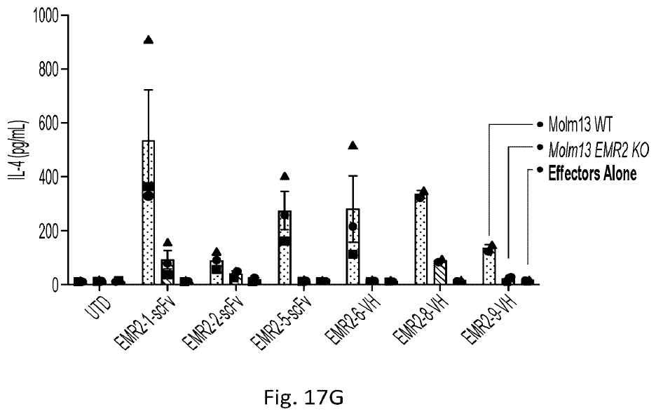

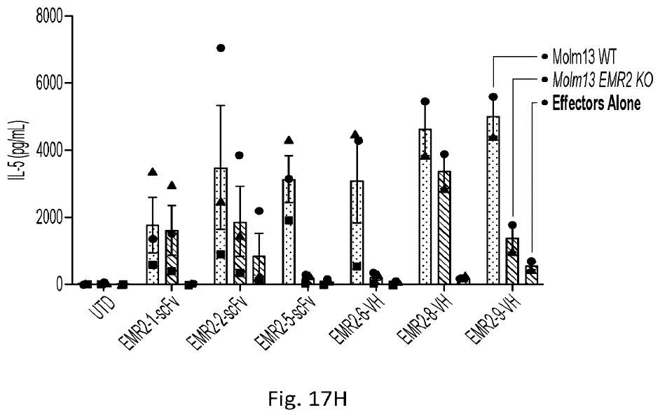

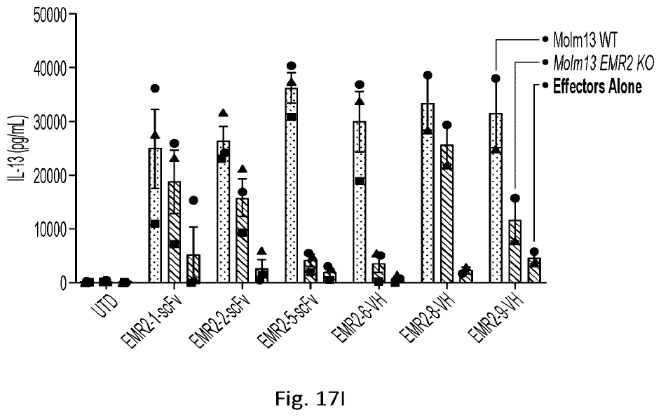

To assess cytokine production by EMR2-directed CAR T cells, effector cells expressing the EMR2-directed CARs were incubated in the presence of target cells (MOLM13 WT or MOLM13 EMR2KO) or effectors alone (CAR alone). Culture supernatant was harvested for detection of cytokine production as measured using either ELISA kits (R&D Systems, Minneapolis, MN, EISA) or a multiplex assay (Meso Scale Discovery, Rockville, MD, EISA). As shown in FIGS. 17A-17I, the EMR2-directed CARs exhibit specific production of IL-2, IFN-α and TNF-α in vitro.

### Generation of EMR2 Deficient Cell Lines

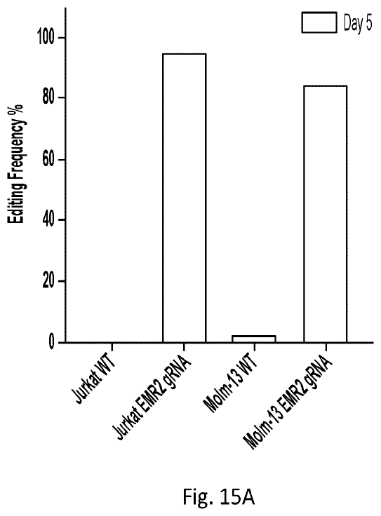

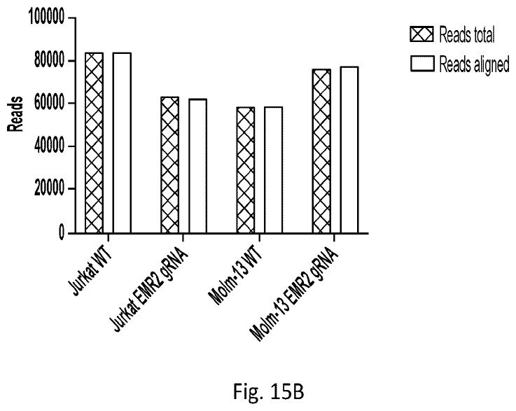

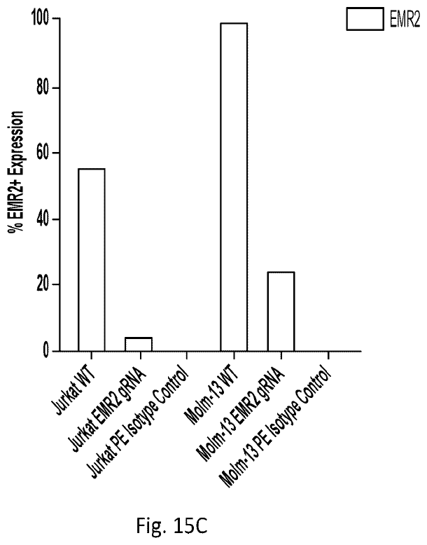

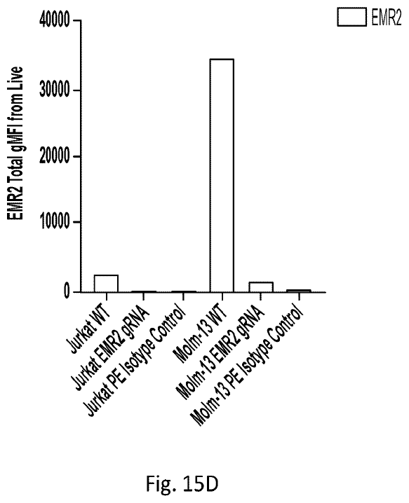

MOLM13 wild type (MOLM13 WT) cells and Jurkat cells were used to generate an EMR2 deficient MOLM13 cell line and EMR2 deficient Jurkat cell line for a true EMR2 “null” target. Briefly, MOLM13 cells were electroporated with a Cas endonuclease and gRNA targeting EMR2 which comprises a spacer sequence of CUUGGCCAAUAACACCAUCC (SEQ ID NO: 104). The editing efficiency was assessed on day 5 following transfection and compared to compared to the corresponding wild-type cells (FIG. 15A). FIG. 15B shows the total and aligned reads for the analysis. Gene editing efficacy was confirmed by assessment of EMR2 surface expression. The wild-type cells displayed expression of EMR2, whereas the gene edited cells Jurkat and MOLM13 cells engineered using the gRNA comprising the spacer sequence provided by SEQ ID NO: 104 had reduced EMR2 expression (FIGS. 15C and 15D).

Gene edited MOLM13 cells having reduced expression of EMR2 are referred to as MOLM13 EMR2KO cells. To analyze the effects of effector cells expressing the EMR2-directed CAR constructs, the CAR-expressing cells were co-cultured with MOLM13-WT or MOLM13 EMR2 KO cells and then assessed by flow cytometry. The percentage of viable target cells in both populations were calculated. As shown in FIGS. 16A and 16B, there were fewer viable target cells of the MOLM13-WT population as compared to the MOLM13 EMR2KO population following 24 hour or 48 hour co-culture.

Activation of the CAR-expressing effector cells was also assessed. For each CAR construct containing an EMR2 binder, the level of effector cell activation was higher in coculture with MOLM13 WT cells as compared to co-culture with EMR2-deficient cells (MOLM13 EMR2KO) or CAR-expressing cells alone (FIGS. 16C and 16D).

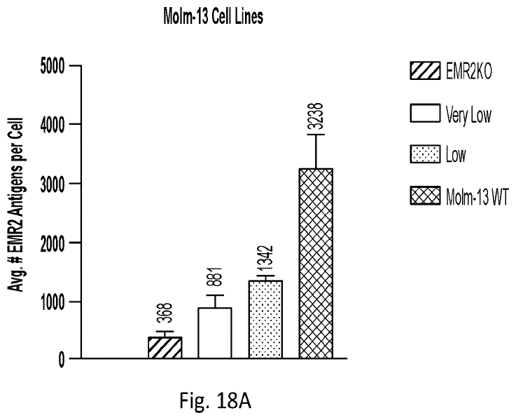

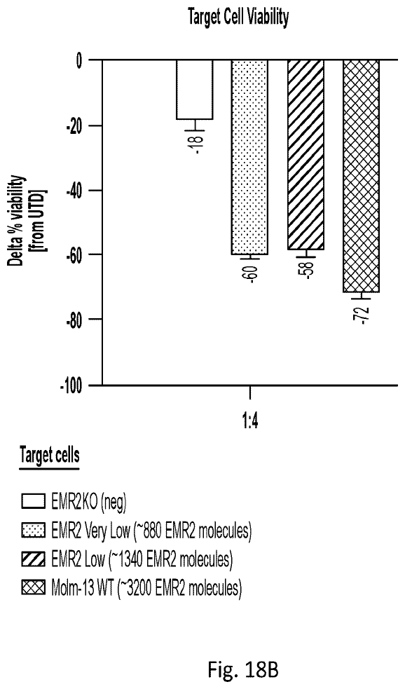

FIGS. 18A-18B show representative results obtained from analyzing the relationship between cell surface levels of EMR2 and EMR2 CAR-induced cytotoxicity. FIG. 18A shows data obtained from flow cytometry analysis of wild-type MOLM13 cells (“Molm-13 WT”), MOLM13 cells engineered using CRISPR/Cas9 and a gRNA to have reduced cell surface levels of EMR2 (“EMR2KO”), and MOLM13 cells engineered using CRISPR/Cas9 to have reduced cell surface levels of EMR2 and express an exogenous copy of the EMR2 gene operably linked to a heterologous promoter (“Low” and “Very Low”). The gRNA used for engineering the EMR2KO, EMR2 Low, and EMR2 Very Low cell populations comprised a spacer sequence of GUGGUACCUGCUGGCUGAGG (SEQ ID NO: 105). FIG. 18B shows reductions in cell viability of wild-type MOLM13 cells (“Molm-13 WT”), MOLM13 cells engineered using CRISPR/Cas9 to have reduced cell surface levels of EMR2 (“EMR2KO”), and MOLM13 cells engineered using CRISPR/Cas9 to have reduced cell surface levels of EMR2 and express an exogenous copy of the EMR2 gene operably linked to a heterologous promoter (“EMR2 Low” and “EMR2 Very Low”) obtained from viability analysis performed after co-culture with Anti-EMR2 Clone EMR2-5-scFv effector cells.

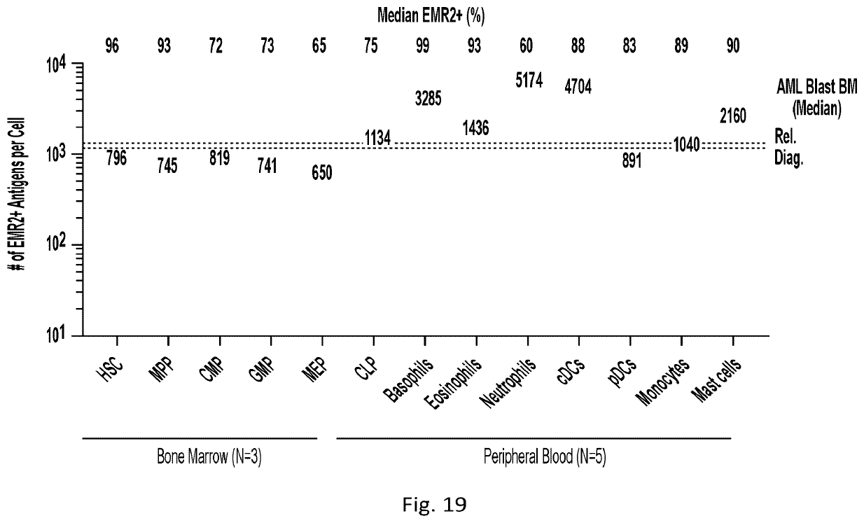

FIG. 19 shows flow cytometric analysis of EMR2 expression on healthy donor bone marrow and peripheral blood. QuantiBrite™ beads (BD Biosciences) were used for antigen quantitation. Hematopoietic stem cell (HSC), multipotent progenitor (MPP), common myeloid progenitor (CMP), granulocyte-monocyte progenitor (GMP), megakaryocytic-erythroid progenitor (MEP), common lymphoid progenitor (CLP), basophils, eosinophils, neutrophils, cDCs, pDCs, monocytes, and mast cells.

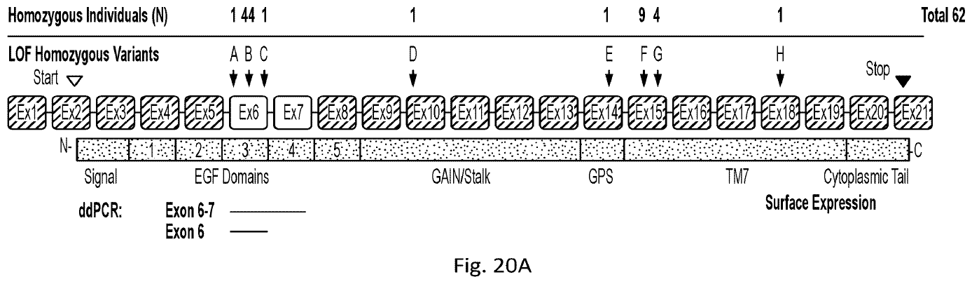

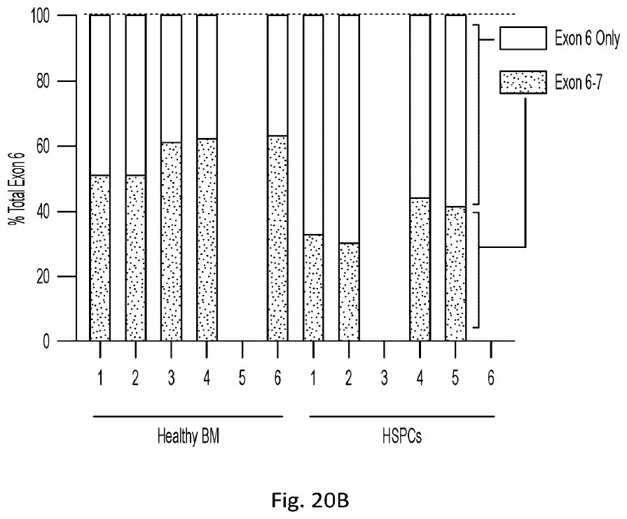

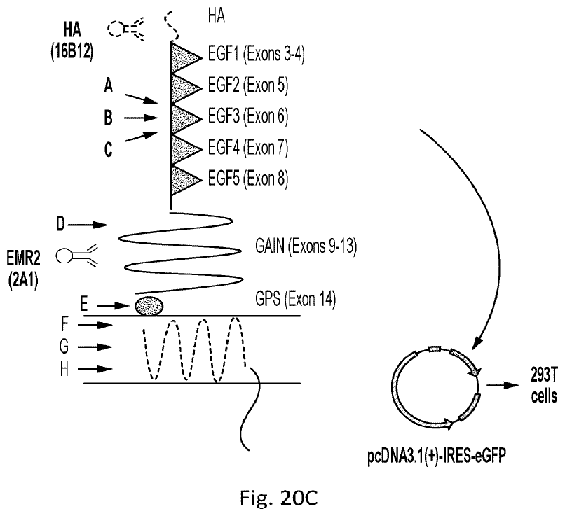

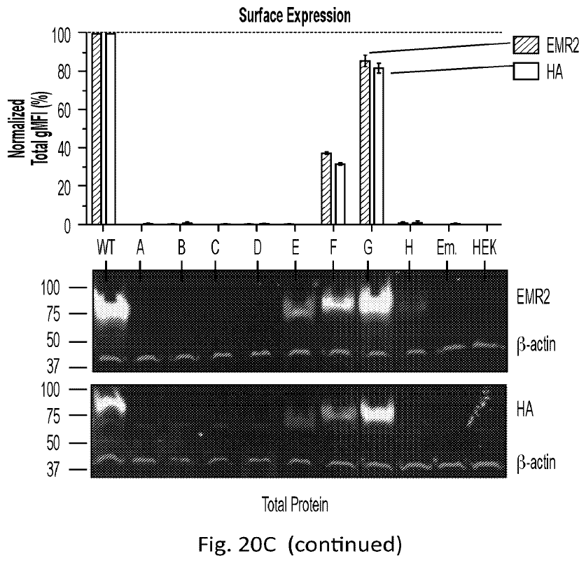

FIGS. 20A-20C show characterization of naturally occurring EMR2 genetic variants. FIG. 20A shows the structure of the EMR2 gene and indicates the locations of loss-of-function (LOF) homozygous variants. The number of individuals identified for homozygous LOF variants at the EMR2 gene regions indicated by “A”, “B”, “C”, “D”, “E”, “F”, “G”, and “H” is shown in the top right. FIG. 20B shows the presence and distribution of exon 6 only and exon 6-7 containing isoforms in samples of healthy bone marrow (BM) and hematopoietic stem and progenitor cells (HSPCs) as analyzed by ddPCR that amplified regions A-H indicated in FIG. 20A. FIG. 20C shows surface and total protein expression of EMR2 variants. Plasmid constructs with EMR2 variant mutations were transfected into 293T cells. EMR2 and HA antibodies were used for surface and total protein detection by flow cytometry and Western blotting, respectively. This data indicates that transplanted EMR2-edited HSCs are capable of differentiation in a subject and also comprise genetic modifications that reduce binding to an EMR2 CAR (see, e.g., FIG. 5B showing EMR2 binders that bind EMR2 variants lacking EGF3, EGF4, and/or EGF5 domains).

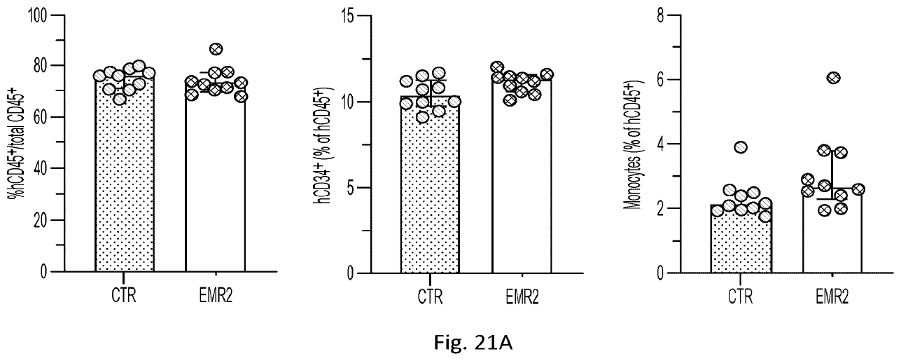

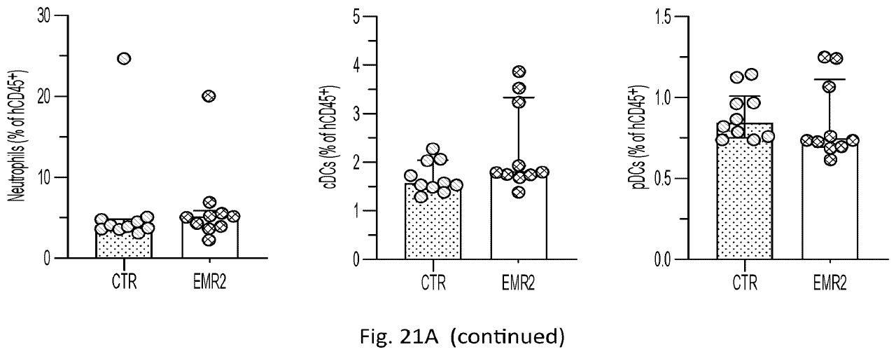

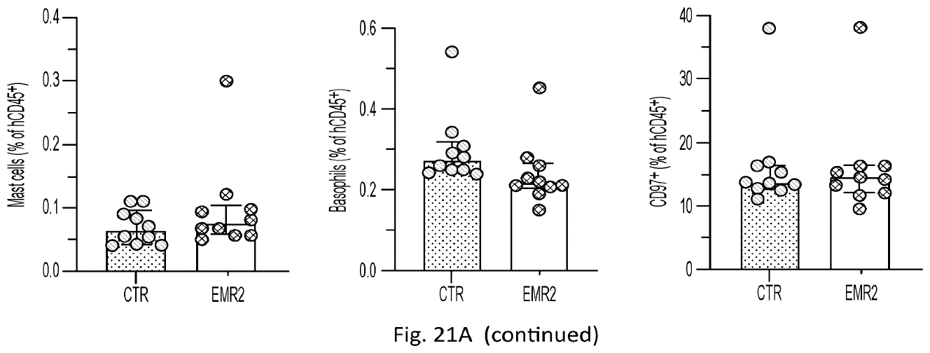

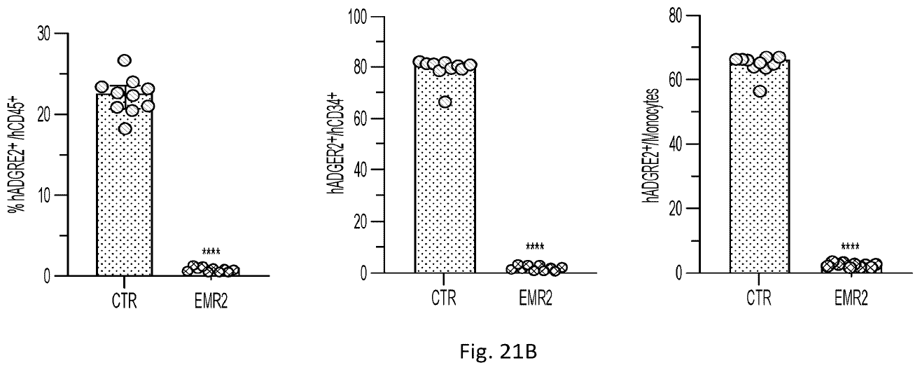

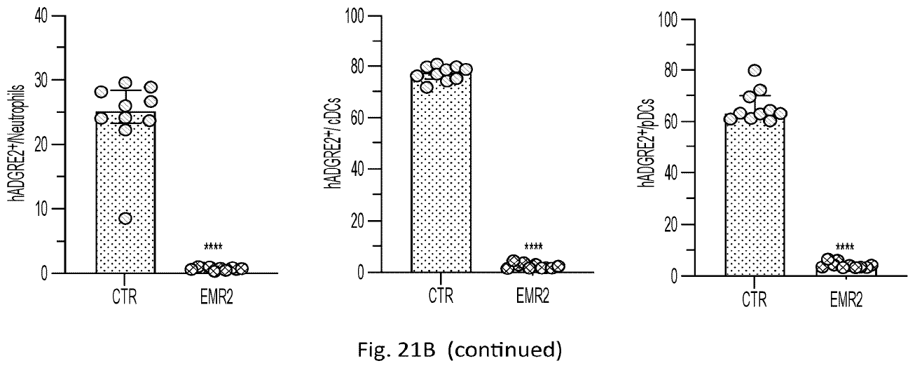

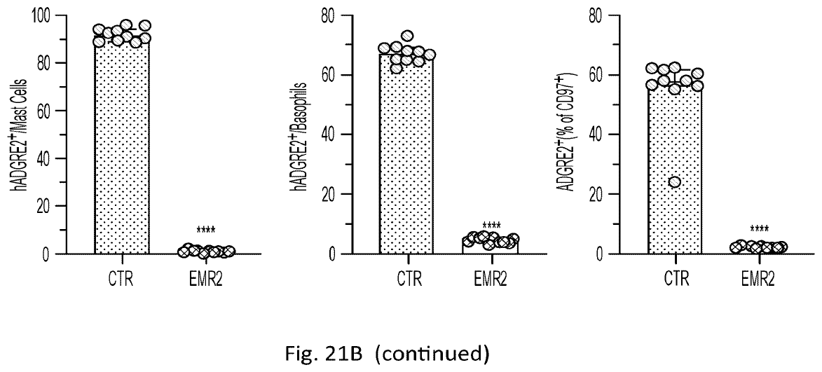

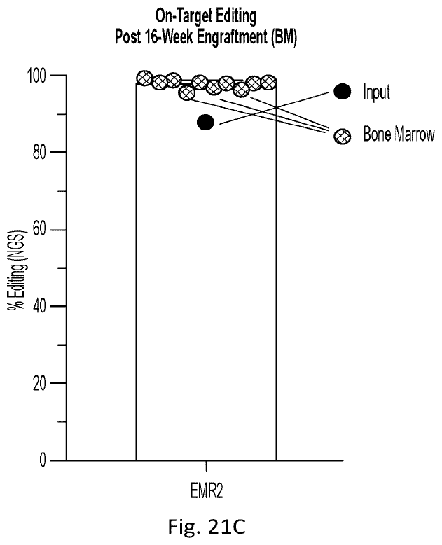

FIGS. 21A-21C show EMR2-edited hematopoietic stem cells engraft long-term, differentiate into multilineages, and persist in vivo. Control (CTR) unedited or ABE guide EMR2-edited CD34+ hHSPCs were xenotransplanted into NOD-scid IL2Rg null (NSG) mice followed by 16-week post-engraftment evaluation of the bone marrow (BM). FIG. 21A shows flow cytometry analysis of bone marrow (BM) indicating comparable levels of total human leukocyte chimerism, human HSPCs and various human hematopoietic lineages, between mice engrafted with CTR or edited cells. This indicates that deletion of EMR2 did not impact HSC function, defined by the ability for long-term engraftment and multilineage reconstitution. FIG. 21B shows significant reduction the EMR2 protein expression in total human cells was observed in the BM. FIG. 21C shows quantification of on-target editing by Next-Gen Sequencing (NGS) of PCR amplicons shows no loss of total editing frequencies after 16 weeks of engraftment at EMR2 locus.

### Example 4. Treatment of Hematologic Disease

An exemplary treatment regimen using the methods, cells, and agents (e.g., a CAR or immune cells expressing a CAR) described herein for AML or MDS is provided. Briefly, a subject having AML or MDS that is a candidate for receiving a hematopoietic stem cell transplant (HSCT) is identified. A suitable HSC donor, e.g., an HLA-matched donor, is identified and HSCs are obtained from the donor, or, if suitable, autologous HSCs from the subject are obtained.

The HSCs so obtained are edited according to the protocols and using the strategies and compositions as described in PCT Publication Nos. WO2023/086422 and WO 2023/043858, e.g., a suitable guide RNA targeting an EMR2 target domain. Briefly, a targeted modification (deletion, truncation, substitution) of EMR2 is introduced via CRISPR gene editing using a suitable guide RNA and a suitable RNA-guided nuclease, e.g., a Cas9 nuclease, resulting in a loss of EMR2 expression in at least 80% of the edited HSC population.

The subject having AML or MDS may be preconditioned according to a clinical standard of care, which may include, for example, infusion of chemotherapy agents e.g., etoposide, cyclophosphamide) and/or irradiation. Depending on the health status of the subject and the status of disease progression in the subject, such pre-conditioning may be omitted, however.

T cells expressing a CAR targeting EMR2 (i.e., EMR2 CAR-T cells) as described herein is administered to the subject. The edited HSCs from the donor or the edited HSCs from the subject are administered to the subject, and engraftment, survival, and/or differentiation of the HSCs into mature cells of the hematopoietic lineages in the subject are monitored. The EMR2 CAR-T cells selectively target and kill EMR2 expressing malignant or pre-malignant cells and may also target some healthy cells expressing EMR2 in the subject but does not target the edited HSCs or their progeny in the subject, as these cells are resistant to targeting and killing by EMR2 CAR-T cells.

The health status and disease progression in the subject is monitored regularly after administration of the EMR2 CAR-T cells and edited HSCs to confirm a reduction in the burden of EMR2-expressing malignant or pre-malignant cells, and to confirm successful engraftment of the edited HSCs and their progeny.

### Example 5. EMR2 CAR Sensitivity

MOLM13 cell lines with variable EMR2 antigen densities were generated by knocking-out the EMR2 gene with CRISPR/Cas9 in MOLM13 wild-type cells and performing lentiviral transduction with full-length EMR2 plasmids under UBC and PGK promoters. Transduced MOLM13 cells were single-cell sorted on the FACSAria II (BD Biosciences) with an anti-EMR2 PE antibody to select for populations with varying expression levels of EMR2. Single-cell colonies were expanded in culture plates with RPMI 1640+10% FBS media, and antigen density was assessed with QuantiBRITE PE beads (BD Biosciences) by flow cytometry (FIG. 18A).

MOLM13 cell line clones with very low EMR2 antigen density (average 881 molecules/cell) and low EMR2 molecules/cell (average 1340 molecules/cell) were co-cultured for 48-hours with Anti-EMR2 Clone EMR2-5-scFv (Table 5) (86.5% CAR+) effector cells from one donor at a 1:4 effector-to-target ratio. MOLM13 WT (average 3200 molecules/cell) and MOLM13 EMR2KO cells (that were used for the generation of the cell line clones) were also included as separate co-culture conditions. As a control, all target cell lines were co-cultured with untransduced (UTD) effector cells from the same donor at a 1:4 effector-to-target ratio (FIG. 18B).

When comparing the viability of the target cells co-cultured with Anti-EMR2 Clone EMR2-5-scFv to cells co-cultured with untransduced (UTD) effector cells, 60% killing of MOLM13 very low expressing cells, 58% killing of MOLM13 low expressing cells, and 72% killing of MOLM13 wild-type cells were observed. Minimal killing of 18% was observed for MOLM13 EMR2KO cells. Overall, Anti-EMR2 Clone EMR2-5-scFv effectively induced cytotoxicity of EMR2-expressing MOLM13 target cells, even those expressing as low as an average of 880 molecules/cell (FIG. 18B).

### INCORPORATION BY REFERENCE

All references, including publications, patent applications, and patents, cited herein are hereby incorporated by reference to the same extent as if each reference were individually and specifically indicated to be incorporated by reference and were set forth in its entirety herein.

### EQUIVALENTS AND SCOPE

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

Articles such as “a,” “an,” and “the” may mean one or more than one unless indicated to the contrary or otherwise evident from the context. Claims or descriptions that include “or” between two or more members of a group are considered satisfied if one, more than one, or all of the group members are present, unless indicated to the contrary or otherwise evident from the context. The disclosure of a group that includes “or” between two or more group members provides embodiments in which exactly one member of the group is present, embodiments in which two or more members of the group are present, and embodiments in which all of the group members are present. For purposes of brevity those embodiments have not been individually spelled out herein, but it will be understood that each of these embodiments is provided herein and may be specifically claimed or disclaimed.

It is to be understood that the invention encompasses all variations, combinations, and permutations in which one or more limitation, element, clause, or descriptive term, from one or more of the claims or from one or more relevant portion of the description, is introduced into another claim. For example, a claim that is dependent on another claim can be modified to include one or more of the limitations found in any other claim that is dependent on the same base claim. Furthermore, where the claims recite a composition, it is to be understood that methods of making or using the composition according to any of the methods of making or using disclosed herein or according to methods known in the art, if any, are included, unless otherwise indicated or unless it would be evident to one of ordinary skill in the art that a contradiction or inconsistency would arise.

Where elements are presented as lists, e.g., in Markush group format, it is to be understood that every possible subgroup of the elements is also disclosed, and that any element or subgroup of elements can be removed from the group. It is also noted that the term “comprising” is intended to be open and permits the inclusion of additional elements or steps. It should be understood that, in general, where an embodiment, product, or method is referred to as comprising particular elements, features, or steps, embodiments, products, or methods that consist, or consist essentially of, such elements, features, or steps, are provided as well. For purposes of brevity those embodiments have not been individually spelled out herein, but it will be understood that each of these embodiments is provided herein and may be specifically claimed or disclaimed.

Where ranges are given, endpoints are included. Furthermore, it is to be understood that unless otherwise indicated or otherwise evident from the context and/or the understanding of one of ordinary skill in the art, values that are expressed as ranges can assume any specific value within the stated ranges in some embodiments, to the tenth of the unit of the lower limit of the range, unless the context clearly dictates otherwise. For purposes of brevity, the values in each range have not been individually spelled out herein, but it will be understood that each of these values is provided herein and may be specifically claimed or disclaimed. It is also to be understood that unless otherwise indicated or otherwise evident from the context and/or the understanding of one of ordinary skill in the art, values expressed as ranges can assume any subrange within the given range, wherein the endpoints of the subrange are expressed to the same degree of accuracy as the tenth of the unit of the lower limit of the range.

In addition, it is to be understood that any particular embodiment of the present invention may be explicitly excluded from any one or more of the claims. Where ranges are given, any value within the range may explicitly be excluded from any one or more of the claims. Any embodiment, element, feature, application, or aspect of the compositions and/or methods described herein, can be excluded from any one or more claims. For purposes of brevity, all of the embodiments in which one or more elements, features, purposes, or aspects is excluded are not set forth.

What is claimed is:

1. An anti-EMR2 antibody, or antigen-binding fragment thereof, comprising an amino acid sequence of any one of SEQ ID NOs: 65, 71, 73, 75, 81, 83, 89, 91, 93, 99, and 101.

2. An anti-EMR2 antibody, or antigen-binding fragment thereof, comprising at least one complementarity determining region (CDR) sequence selected from DSL, DGL, or SEQ ID NOs: 12-28 and 30-58.

3. The anti-EMR2 antibody, or antigen-binding fragment thereof, of claim 2, comprising three CDR sequences selected from DSL, DGL, or SEQ ID NOs: 12-28 and 30-58.

4. The anti-EMR2 antibody, or antigen-binding fragment thereof, of claim 2, comprising six CDR sequences selected from DSL, DGL, or SEQ ID NOs: 12-28 and 30-58.

5. The anti-EMR2 antibody, or antigen-binding fragment thereof, of claim 2, comprising three heavy chain CDR sequences selected from any of DSL, DGL, or SEQ ID NOs: 15-17, 21-28, 33-38, 42-50, and 54-58.

6. An anti-EMR2 antibody, or antigen-binding fragment thereof, comprising a CDR1, CDR2, and CDR3 selected from DSL, DGL, or SEQ ID NOs: 15-17, 21-28, 33-38, 42-50, and 54-58.

7. An anti-EMR2 antibody, or antigen-binding fragment thereof, comprising at least one CDR that is at least 85%, 90%, 91%, 92%, 93%, 94%, 95%, 96%, 97%, 98%, 99%, or 100% identical to a CDR (e.g., CDR1, CDR2, and/or CDR3) selected from DSL, DGL, or SEQ ID NOs: 15-17, 21-28, 33-38, 42-50, and 54-58.

8. An anti-EMR2 antibody, or antigen-binding fragment thereof, comprising a heavy chain variable region (VH) comprising:
a CDR1 provided by any one of SEQ ID NOs: 15, 21, 24, 27, 33, 36, 42, 45, 48, 54, and 57;
a CDR2 provided by any one of SEQ ID NOs: 16, 22, 25, 28, 34, 37, 43, 46, 49, 55, and 58;
a CDR3 provided by any one of DSL, DGL, or SEQ ID NOs: 17, 23, 26, 35, 38, 44, 47, 50, and 56; and
a light chain variable region (VL) comprising:
a CDR1 provided by any one of SEQ ID NOs: 12, 18, 30, 39, and 51,
a CDR2 provided by any one of SEQ ID NOs: 13, 19, 31, 40, and 52, and
a CDR3 provided by any one of SEQ ID NOs: 14, 20, 32, 41, and 53.

9. The anti-EMR2 antibody, or antigen-binding fragment thereof, of claim 8 comprising:
a heavy chain variable region (VH) that is at least 85%, 90%, 91%, 92%, 93%, 94%, 95%, 96%, 97%, 98%, 99%, or 100% identical to any one of SEQ ID NOs: 63, 69, 73, 75, 79, 83, 87, 91, 93, 97, and 101; and
a light chain variable region (VL) that is at least 85%, 90%, 91%, 92%, 93%, 94%, 95%, 96%, 97%, 98%, 99%, or 100% identical to any one of SEQ ID NOs: 61, 67, 77, 85, and 95.

10. The anti-EMR2 antibody, or antigen-binding fragment thereof, of claim 9 comprising a heavy chain variable region (VH) set forth as any one of SEQ ID NOs: 63, 69, 73, 75, 79, 83, 87, 91, 93, 97, and 101, and a light chain variable region (VL) set forth as any one of SEQ ID NOs: 61, 67, 77, 85, and 95.

11. An anti-EMR2 antibody, or antigen-binding fragment thereof, comprising a heavy chain variable region (VH) comprising:
a CDR1 provided by any one of SEQ ID NOs: 15, 21, 24, 27, 33, 36, 42, 45, 48, 54, and 57;
a CDR2 provided by any one of SEQ ID NOs: 16, 22, 25, 28, 34, 37, 43, 46, 49, 55, and 58; and
a CDR3 provided by any one of DSL, DGL, or SEQ ID NOs: 17, 23, 26, 35, 38, 44, 47, 50, and 56.

12. The anti-EMR2 antibody, or antigen-binding fragment thereof, of claim 11, wherein the VH comprises an amino acid sequence of any one of SEQ ID NOs: 63, 69, 73, 75, 79, 83, 87, 91, 93, 97, and 101.

13. An anti-EMR2 antibody, or antigen-binding fragment thereof, comprising a heavy chain variable region (VH) that is at least 85%, 90%, 91%, 92%, 93%, 94%, 95%, 96%, 97%, 98%, 99%, or 100% identical to any one of SEQ ID NOs: 63, 69, 73, 75, 79, 83, 87, 91, 93, 97, and 101.

14. The anti-EMR2 antibody or antigen-binding fragment thereof, of any one of claims 1-13, wherein the antibody, or antigen-binding fragment thereof, is a monoclonal antibody, or antigen-binding fragment thereof.

15. The anti-EMR2 antibody, or antigen-binding fragment thereof, of any one of claims 1-14, wherein the antibody, or antigen-binding fragment thereof, is a human antibody or a humanized antibody, or antigen-binding fragment thereof.

16. An anti-EMR2 antibody, or antigen-binding fragment thereof, that competes with the antibody, or antigen-binding fragment thereof, of any one of claims 1-15.

17. The anti-EMR2 antibody, or antigen-binding fragment thereof, of any one of claims 1-16, wherein the antibody, or antigen-binding fragment thereof, comprises a CH1 constant domain, a CH2 constant domain, and a CH3 constant domain.

18. The anti-EMR2 antibody, or antigen-binding fragment thereof, of claim 17, wherein the antibody, or antigen-binding fragment thereof, comprises an amino acid sequence of any one of SEQ ID NOs: 65, 71, 73, 75, 81, 83, 89, 91, 93, 99, and 101.

19. The anti-EMR2 antibody, or antigen-binding fragment thereof, of claim 18 further comprising a linker.

20. The anti-EMR2 antibody, or antigen-binding fragment thereof, of any one of claims 1-19, wherein said antibody is of the IgG1-, IgG2-, IgG3 or IgG4-type.

21. The anti-EMR2 antibody, or antigen-binding fragment thereof, of any one of claims 1-19, wherein the antibody, or antigen-binding fragment thereof, is a heavy chain antibody.

22. The anti-EMR2 antibody, or antigen-binding fragment thereof, of any one of claims 1-19, wherein the antibody, or antigen-binding fragment thereof, is a camelid antibody.

23. A chimeric antigen receptor (CAR) comprising any one of the antibodies, or antigen-binding fragment thereof, of any one of claims 1-22.

24. A cell expressing the CAR of claim 23.

25. The cell of claim 24, wherein the cell is an immune effector cell.

26. The cell of claim 24 or 25, wherein the cell is a lymphocyte.

27. The cell of any one of claims 24-26, wherein the cell is a T-cell.

28. The cell of any one of claims 24-26, wherein the cell is a Natural Killer (NK) cell.

29. A pharmaceutical composition comprising the antibody, or antigen-binding fragment thereof, of any one of claims 1-22, the chimeric antigen receptor of claim 23, or the cell of any one of claims 24-28, and a pharmaceutically acceptable excipient.

30. A nucleic acid comprising a nucleotide sequence encoding the antibody, or antigen-binding fragment thereof, of any one of claims 1-22, or the chimeric antigen receptor of claim 23.

31. The nucleic acid of claim 30 comprising a nucleotide sequence of any one of SEQ ID NOs: 64, 70, 72, 74, 80, 82, 88, 90, 92, 98, and 100.

32. A vector comprising the nucleic acid of claim 30 or 31.

33. A cell comprising the nucleic acid of claim 30 or 31 or the vector of claim 32.

34. The cell of claim 33, wherein the cell is an immune cell.

35. The cell of claim 34, wherein the immune cell is a T cell, a Natural Killer (NK) cell, a cytotoxic T lymphocyte (CTL), or a regulatory T cell.

36. A method of producing an antibody, or antigen-binding fragment thereof, comprising culturing the cell of any one of claims 33-35 under conditions suitable for expression of the antibody or antigen-binding fragment thereof.

37. A method of treating a disease or disorder associated with expression of EMR2 or a variant thereof, the method comprising administering to a subject in need thereof a therapeutically effective amount of an agent that targets EMR2, wherein the agent comprises the antibody, or antigen-binding fragment thereof, of any one of claims 1-22, the CAR of claim 23, or the cell of any one of claims 24-28 or 33-35.

38. The method of claim 37, wherein the disease or disorder associated with expression of EMR2 or a mutant variant thereof is a hematopoietic malignancy or a premalignancy.

39. The method of claim 38, wherein the hematopoietic malignancy or premalignancy is acute myeloid leukemia (AML) or myelodysplastic syndrome (MDS).

40. The method of any one of claims 37-39, further comprising administering to the subject an effective amount of a chemotherapeutic agent or an oncolytic therapeutic agent.

41. The method of any one of claims 37-40, further comprising administering a genetically engineered cell, or cell population thereof, comprising a genetic modification in a gene encoding EMR2, wherein the genetic modification results in reduced or eliminated expression of EMR2 or expression of a variant of EMR2 as compared to EMR2 expressed by wild-type cells of the same cell type that do not harbor the genetic modification in the EMR2 gene.

42. The method of claim 41, wherein administration of the agent targeting EMR2 occurs simultaneously or in temporal proximity with administration of the genetically engineered cell, or cell population thereof.

43. The method of claim 41, wherein administration of the agent targeting EMR2 occurs after administration of the genetically engineered cell, or cell population thereof.

44. The method of claim 41, wherein administration of the agent targeting EMR2 occurs before administration of the genetically engineered cell, or cell population thereof.

45. The method of any one of claims 41-44, wherein the genetically engineered cell is a stem cell.

46. The method of any one of claims 41-45, wherein the genetically engineered cell is a hematopoietic cell.

47. The method of any one of claims 41-46, wherein the genetically engineered cell is a hematopoietic stem cell.

48. The method of any one of claims 41-47, wherein the genetically engineered cell is a hematopoietic progenitor cell.

49. The method of any one of claims 41-48, wherein the genetically engineered cell is derived from the subject in need thereof.

50. The method of any one of claims 41-48, wherein the genetically engineered cell is obtained from an allogenic donor.

51. The method of any one of claims 41-50, wherein the agent is a genetically engineered cell that is obtained, or a descendent of a cell that is obtained, from the subject in need thereof.

52. The cell of any one of claims 24-28 or 33-35, wherein the cell comprises a genetic modification in a gene encoding EMR2, wherein the genetic modification results in reduced or eliminated expression of EMR2 or expression of a variant of EMR2 as compared to EMR2 expressed by wild-type cells of the same cell type that do not harbor the genetic modification in the EMR2 gene.

53. A method comprising contacting a cell the antibody, or antigen-binding fragment thereof, of any one of claims 1-22, the chimeric antigen receptor of claim 23, the cell of any one of claims 24-28, the nucleic acid of claim 30, or the vector claim 31.
